(19) United States
(12) Patent Application Publication
(10) Pub. No.: US 2017/0333560 A1
(43) Pub. Date: Nov. 23, 2017

(54) Pharmaceutical Composition That Includes A Neutral Carrier Treated With Energy And Related Methods Of Treatment

(71) Applicant: Oleg Iliich Epshtein, Moscow (RU)

(72) Inventor: Oleg Iliich Epshtein, Moscow (RU)

(21) Appl. No.: 15/598,088

(22) Filed: May 17, 2017

(30) Foreign Application Priority Data
May 17, 2016 (RU) 2016119055

(51) Int. Cl. A61K 41/00; A61K 47/26; A61B 5/0478

(52) U.S. Cl. CPC A61K 41/0004; A61B 5/0478; A61K 47/26

(57) ABSTRACT

The present invention provides a pharmaceutical composition that includes a neutral carrier treated with energy proportional to biopotential of human brain or nervous tissue. Preferably, the neutral carrier is obtained from initial liquid neutral carrier which after the energy treatment is subjected to multiple consecutive dilution combined with external shaking of each dilution in accordance with homeopathic technology. Preferably, the initial liquid carrier is treated with electrical current by passing electricity through at least two electrodes, which current is proportional in amplitude to biopotential of human brain.

A variety of methods of treatment is provided, including methods of treatment of psychosis, depression, attention deficit disorder, and other conditions of central nervous system, including a number of psychiatric conditions.

A device for passing electricity through the neutral liquid carrier is also provided.

### BACKGROUND

The brain is a part of the central nervous system. It is the regulatory center for all functions in the body. Brain cells are neurons responsible for processing and transmitting information by generating electrical signals. Brain bioelectrical activity is a complex oscillatory electrical (electromagnetic) process resulting from summated physicochemical processes in neurons which can be recorded as an electro-encephalogram (EEG)—a record of brain electric potentials picked up by electrodes attached to the scalp overlying the brain. Electrical activity recordings of individual neurons reflect their functional activity in information processing and transmission, whereas the overall EEG pattern represents the functional activity of the whole brain. The pattern of bioelectrical activity depends on the functional state of the brain (i.e., sleep or waking state, mental or physical activity, etc.) and the nature of processes that elicit elementary potentials (spontaneous or evoked activity).

It is known that the EEG pattern is a complex rhythmical trace representing the bioelectrical activity of the brain, and generated by the interaction of its numerous regulatory systems responsible for the highest level of control and integration in the body. There is evidence from both experimental and clinical studies demonstrating a link between certain changes in the EEG pattern and processes in relevant brain structures.

In the prior art, a means to produce an effect on the body is known which consists of a neutral liquid carrier treated by exposure to induced energy oscillations with frequencies and amplitudes proportional to those of biopotentials recorded from the body, and then potentized by repeated serial dilution according to the homeopathic method (RU 2140253 C1, A 61 J 3/00, 1999). The energy treatment is carried out by exciting mechanical acoustic vibrations in the initial neutral carrier using, for instance, an electrostrictive element or a membrane, or by generating electromagnetic waves, for example by means of an electromagnetic coil, which is not capable of effectively translating (recording) onto the carrier the bioenergy information which adequately reflects the individual features of brain bioenergetics.

In the prior art, a device is also known which is used for recording electro-encephalograms (the EEG machine)—records of electric potential changes from the brain which are picked up by electrodes attached to a person's scalp and connected to a circuit of amplifiers, filters and recorders (see L. R. Zenkov, ‘Clinical EEG with epileptology elements’ (in Russian), 3 ed., Moscow, MEDpress-inform, 2004, 368p.). However, this device does not comprise any elements to facilitate an energy treatment of the neutral liquid carrier.

### SUMMARY

While the invention is not limited by any specific theory, it has been surprisingly discovered by the inventors of the present patent application that a neutral carrier treated with energy proportional to biopotentials of a human brain or nervous tissue has therapeutic effect on a variety of deceases and conditions of the central nervous system, including several psychiatric conditions.

In the preferred embodiment of the invention, an initial liquid carrier, for example, distilled water or water-alcohol mixture, is treated by passing electric current through the liquid carrier, wherein the current is modulated in accordance with biopotentials obtained from EEG. Preferably, at least two electrodes are placed in the initial liquid carrier and the liquid carrier is treated with electricity proportional in amplitude to biopotentials of the human brain obtained by EEG. Preferably, the initial liquid carrier may then be subjected to multiple consecutive dilutions in accordance with homeopathic technology to potentiate the energy signature imprinted by the energy treatment. The liquid carrier maybe used in the methods of treatment of the present invention directly in the liquid form or a solid neutral carrier maybe impregnated with the liquid carrier.

The initial (starting) carrier is preferably distilled or purified (special purification grade) water or water-alcoholic solution with a maximum conductivity of 0.1 mS/m at 25° C., or an activated (potentized) carrier containing distilled or purified (special purification grade) water or water-alcoholic solution with a maximum conductivity of 0.1 mS/m at 25° C., which has been treated by repeated serial dilution in an aqueous or aqueous-alcoholic diluent in combination with external mechanical impact.

In one variant, the difference of potentials being applied to the pair of electrodes in the initial carrier is proportional in the amplitude to the difference of biopotentials recorded in a bipolar derivation consisting of active recording electrodes placed at different locations on the surface of the human scalp.

Preferably, the difference of potentials being applied to the pair of electrodes in the initial carrier is proportional in the amplitude to the difference of biopotentials recorded in a referential derivation consisting of an active scalp electrode and a reference electrode located on the ear-lobe.

Also, the scalp electrode may be referenced to a pair of short-circuited electrodes placed on either ear-lobe.

In another variant, the difference of potentials being applied to at least two electrodes in the initial carrier is proportional in the amplitude to an amplified summed or averaged signal formed by adding up the differences of bioelectric potentials from referential derivations of recording electrodes that contribute to different channels from different scalp locations.

Preferably, the difference of potentials being applied to at least two electrodes in the initial carrier is proportional in the amplitude to an amplified summed or averaged signal formed by adding up the differences of bioelectric potential from a series of referential derivations of recording electrodes that contribute to different channels from different scalp locations.

Alternatively, the energy treatment of the initial carrier is accomplished by exposing it to an electric current caused by differences of electric potentials proportional in amplitude to the differences of biopotentials of referential derivations which are applied to a set of electrodes in the initial carrier, one of which is potential from the reference ear-lobe electrode and the others receive amplified potentials from the active electrodes positioned at different scalp locations.

Further, wherein the energy treatment of the initial carrier is accomplished by exposing it to an electric current caused by a difference of potentials being applied to at least two electrodes in the initial carrier and which is proportional in amplitude to a summed or averaged differences in biopotential recorded at different sites of the brain—on the surface of the scalp of a patient—the individual to be exposed to the therapeutic effect.

Alternatively, the potential difference being applied to at least two electrodes in the initial carrier is proportional in the amplitude to an amplified summed or averaged signal formed by adding up the differences of bioelectric potential from referential derivations of active electrodes contributing to different channels from different locations on the scalp of a donor—the individual the biopotential recordings from whom are used in preparing the drug for treating a patient.

Alternatively, the energy treatment of the initial carrier is accomplished by exposing it to an electric current caused by a difference of potentials being applied to at least two electrodes in the initial carrier and which is proportional in the amplitude to a combined summed or averaged difference in biopotentials recorded in individual referential derivations from different sites of the brain—on the surface of the scalp of one or more donors.

Preferably, the energy treatment of the initial carrier is accomplished by exposing it to an electric current caused by a difference of potentials being applied to at least two electrodes in the initial carrier and which is proportional in amplitude to a combined summed or averaged difference in biopotential recorded in individual referential derivations from different sites of the brain—on the surface of the scalp of a patient and one or more donors.

Alternatively, the energy treatment of the initial carrier is accomplished by exposing it to an electric current caused by a difference of potentials being applied to at least two electrodes in the initial carrier and which is proportional in amplitude to a combined summed or averaged difference in biopotential recorded in individual referential derivations from different sites of the brain—of two or more donors.

Alternatively, at least two separately prepared drugs are administered, each of which is prepared by exposing said carrier to the energy treatment using the potential difference applied to electrodes in the initial carrier, said difference being proportional in amplitude to the biopotential difference of electrodes recording from different sites of the brain of one or more donors.

Alternatively, the drug administered contains a mixture of at least two ingredients, one of which is prepared by exposing said carrier to the energy treatment using the potential difference applied to electrodes in the initial carrier, said difference being proportional in amplitude to the biopotential difference between electrodes recording from different sites of the brain of a patient, and the other is obtained by exposing said carrier to the energy treatment using the potential difference applied to electrodes in the initial carrier, said difference being proportional in amplitude to the biopotential difference of electrodes recording from different sites of the brain of one or more donors.

Alternatively, the drug administered contains a mixture of at least two ingredients, each of which is prepared by exposing said carrier to the energy treatment using the potential difference applied to electrodes in the initial carrier, said difference being proportional in amplitude to the biopotential difference of electrodes recording from different sites of the brain of different donors.

Alternatively, in one embodiment of the claimed method, the energy treatment of the neutral carrier may be accomplished by exposing it to an electric current caused by a difference of potentials being applied to electrodes placed in the initial carrier, or by exciting in said carrier acoustic vibrations with amplitudes and frequencies proportional to those of the biopotential variations recorded, which can be done, for instance, by means of an electrostrictive element or the membrane of an electro-acoustic converter, or by generating electromagnetic waves (for example, using an electromagnetic coil) with amplitudes and frequencies proportional to those of the biopotential variations recorded.

In one aspect, the invention provides a method of treating disorders of central nervous system or mental decease by administering the pharmaceutical composition of one of the embodiments of the invention. In one embodiment of this aspect of the invention, the decease or condition is t impairment of nervous system functions, including but not limited to movement disorders, such as disturbances observed in a cerebrovascular accident—stroke, sequelae of cerebrovascular accident, parkinsonism/Parkinson's disease, amyotrophic lateral sclerosis, myasthenia, cerebral palsy, multiple sclerosis, sensory impairments, such as the sensory impairments observed in multiple sclerosis, a cerebrovascular accident, polyneuropathy, or autonomic disturbances—dysautonomia, or coordination disturbances, such as coordination disturbances observed in multiple sclerosis, the coordination disturbances are those observed in polyneuropathy, cerebrovascular disease, such as impairments of cognitive function, including but not limited to dementia, Huntington's chorea, atherosclerosis; vascular dementia, or the impairment of nervous system functions is episodic and paroxysmal disorders, such as due to epilepsy, epileptic seizure, common migraine.

In another embodiment of the method aspect of the invention, the disease or disorder of the nervous system is mental and behavioral disorders, including but not limited to disorders with psychotic symptoms, such as disorders with productive psychotic symptoms, for example schizophrenia, paranoid or hallucinatory-delusional syndrome, paranoid or hallucinatory-delusional syndrome in organic brain disease, delirium; or disorders with negative (deficit-type) syndrome, such as emotional and volitional disturbances in schizophrenia, epileptic personality disorders, psycho-organic personality disorders, disorders with neurotic symptoms, such as generalized anxiety disorder, psychological stress disorder, pseudoneurotic disorder in organic brain disease, pseudoneurotic disorder in chronic somatic disease, sleep disturbance, depression

In another embodiment, the mental and behavioral disorders are behavioral disorders, including but not limited to behavioral disorders of childhood, attention deficit hyperactivity disorder, phobic anxiety disorder—fears and anxiety disorders specific to childhood, infantile autism, pathological personality development, psychopathic behavior due to perinatal injury to the nervous system, cognitive impairments in children, or the behavioral disorders are disorders of adult behavior, including but not limited to behavioral disorders due to use of alcohol, or the behavioral disorders are disorders associated with mental retardation—delayed psychological development in children (developmental delay), including but not limited to specific developmental disorders of speech and language, specific developmental disorders of scholastic skills, mixed specific disorders of psychological development, specific developmental disorder of motor function, or the behavioral disorders are disorders associated with impairments of cognitive function in adults, including but not limited to chronic cerebrovascular diseases/chronic vascular disorders of the brain, dementia in Alzheimer's disease, dementia in other diseases.

In another embodiment of the method aspect of the present invention, the disease or disorder of the nervous system is sleep disturbance, including but not limited to sleep disturbance in neurotic and pseudoneurotic disorders, sleep disturbance in chronic cerebrovascular disease, sleep disturbance in alcohol dependence, sleep disturbance in a stress-related reaction, sleep disturbance is t in healthy individuals, sleep disturbance in somatic diseases,

In yet another embodiment, the nervous or mental disease is impairments of cognitive function, including but not limited to developmental delay in children.

In yet another embodiment, the nervous or mental disease is mood disorders/disturbances, including but not limited to depression in endogenous mental disorders, including schizophrenia, and organic brain disease, manic states in endogenous mental disorders, including schizophrenia, organic brain disease, depression in neurotic and pseudoneurotic disorders, post-traumatic or reactive (situational) depression, depressions in chronic cerebrovascular disease in the elderly.

In yet another embodiment, the nervous or mental disease of the nervous system is neurotic and pseudoneurotic disorders.

In yet another embodiment, the nervous or mental disease of the nervous system is unstable remission in mental disorders.

In yet another embodiment, the nervous or mental disease is a disorder of the autonomic nervous system, which may be a primary disorder or a somatic symptom disorder.

In another embodiment, the nervous or mental disease is a disorder of the peripheral nervous system, including but not limited to vertebrogenic pain, and dorsopathy.

In yet another embodiment, the nervous or mental disease is Down syndrome.

In yet another embodiment, the nervous or mental disease is Huntington's disease.

In yet another embodiment, the nervous or mental disease is unstable remission in neurological disorders.

In accordance with one aspect, the present invention provides a method of treating alcoholism in a human patient comprising administering an effective amount of the pharmaceutical composition in accordance with an embodiment of the present invention to the patient in need thereof. Preferably, after energy treatment of the initial neutral carrier, the resulting initial carrier is subjected to multiple consecutive dilutions coupled with external mechanical treatment in accordance with homeopathic technology. After the dilution, the resulting carrier is impregnated onto solid neutral carrier. Etiology of alcoholism is described for example in https://en.wikipedia.org/wiki/Alcoholism, which is incorporated herein by reference. Any number of dilutions maybe used in this method of the invention, however C200 dilution is preferred. Preferably, the pharmaceutical composition is prepared by using brain biopotentials of five healthy donors.

In accordance with another aspect, the present invention provides a method of treating psychosis in a human patient by administering an effective of the pharmaceutical composition in accordance with an embodiment of the present invention to the patient in need thereof. Preferably, after energy treatment of the initial neutral carrier, the resulting initial carrier is subjected to multiple consecutive dilutions coupled with external mechanical treatment in accordance with homeopathic technology. After the dilution, the resulting carrier is impregnated onto solid neutral carrier. Etiology of psychosis is described for example in https://en.wikipedia.org/wiki/Psychosis, which is incorporated herein by reference. Any number of dilutions maybe used in this method of the invention, however C200 dilution is preferred. Preferably, the pharmaceutical composition is prepared by using brain biopotentials of five healthy donors. In one variant of this embodiment of the invention, psychosis is the result of schizophrenia.

In accordance with another aspect, the present invention provides a method of treating posttraumatic stress disorder in a human patient by administering an effective of the pharmaceutical composition in accordance with an embodiment of the present invention to the patient in need thereof. Preferably, after energy treatment of the initial neutral carrier, the resulting initial carrier is subjected to multiple consecutive dilutions coupled with external mechanical treatment in accordance with homeopathic technology. After the dilution, the resulting carrier is impregnated onto solid neutral carrier. Etiology of PTSD is described for example in https://en.wikipedia.org/wiki/Posttraumatic_stress_disorder, which is incorporated herein by reference. Any number of dilutions maybe used in this method of the invention, however C200 dilution is preferred. Preferably, the pharmaceutical composition is prepared by using brain biopotentials of five healthy donors.

In accordance with another aspect, the present invention provides a method of treating depression in a human patient by administering an effective of the pharmaceutical composition in accordance with an embodiment of the present invention to the patient in need thereof. Preferably, after energy treatment of the initial neutral carrier, the resulting initial carrier is subjected to multiple consecutive dilutions coupled with external mechanical treatment in accordance with homeopathic technology. After the dilution, the resulting carrier is impregnated onto solid neutral carrier. Etiology of depression is described for example in https://en.wikipedia.org/wiki/Depression, which is incorporated herein by reference. The pharmaceutical composition of the present invention can be used to treat depression of any ethiology, including depression in PTSD and in bipolar disorder. Any number of dilutions maybe used in this method of the invention, however C200 dilution is preferred. Preferably, the pharmaceutical composition is prepared by using brain biopotentials of five healthy donors.

In accordance with another aspect, the present invention provides a method of treating attention deficit disorder in a human patient by administering an effective of the pharmaceutical composition in accordance with an embodiment of the present invention to the patient in need thereof. Preferably, after energy treatment of the initial neutral carrier, the resulting initial carrier is subjected to multiple consecutive dilutions coupled with external mechanical treatment in accordance with homeopathic technology. After the dilution, the resulting carrier is impregnated onto solid neutral carrier. Etiology of ADD/ADHD is described for example in https://en.wikipedia.org/wiki/Attention_deficit_hyperactivity_disorder, which is incorporated herein by reference. Any number of dilutions maybe used in this method of the invention, however C200 dilution is preferred. Preferably, the pharmaceutical composition is prepared by using brain biopotentials of five healthy donors.

In accordance with another aspect, the present invention provides a method of treating cerebral palsy in a human patient by administering an effective of the pharmaceutical composition in accordance with an embodiment of the present invention to the patient in need thereof. Preferably, after energy treatment of the initial neutral carrier, the resulting initial carrier is subjected to multiple consecutive dilutions coupled with external mechanical treatment in accordance with homeopathic technology. After the dilution, the resulting carrier is impregnated onto solid neutral carrier. Etiology of cerebral palsy is described for example in https://en.wikipedia.org/wiki/Cerebral_palsy, which is incorporated herein by reference. Any number of dilutions maybe used in this method of the invention, however C200 dilution is preferred. Preferably, the pharmaceutical composition is prepared by using brain biopotentials of five healthy donors. Preferably, the patient is a child.

In accordance with another aspect, the present invention provides a method of treating consequences of a stroke in a human patient by administering an effective of the pharmaceutical composition in accordance with an embodiment of the present invention to the patient in need thereof. Preferably, after energy treatment of the initial neutral carrier, the resulting initial carrier is subjected to multiple consecutive dilutions coupled with external mechanical treatment in accordance with homeopathic technology. After the dilution, the resulting carrier is impregnated onto solid neutral carrier. Etiology of stroke is described for example in https://en.wikipedia.org/wiki/Stroke, which is incorporated herein by reference. Any number of dilutions maybe used in this method of the invention, however C200 dilution is preferred. Preferably, the pharmaceutical composition is prepared by using brain biopotentials of five healthy donors.

In accordance with another aspect, the present invention provides a method of treating anxiety in a human patient by administering an effective of the pharmaceutical composition in accordance with an embodiment of the present invention to the patient in need thereof. Preferably, after energy treatment of the initial neutral carrier, the resulting initial carrier is subjected to multiple consecutive dilutions coupled with external mechanical treatment in accordance with homeopathic technology. After the dilution, the resulting carrier is impregnated onto solid neutral carrier. Etiology of anxiety is described for example in https://en.wikipedia.org/wiki/Anxiety, which is incorporated herein by reference. Anxiety of any etiology may be treated, including generalized anxiety. Any number of dilutions maybe used in this method of the invention, however C200 dilution is preferred. Preferably, the pharmaceutical composition is prepared by using brain biopotentials of five healthy donors.

In accordance with another aspect, the present invention provides a method of treating speech disorders in a human patient by administering an effective amount of the pharmaceutical composition in accordance with an embodiment of the present invention to the patient in need thereof. Preferably, after energy treatment of the initial neutral carrier, the resulting initial carrier is subjected to multiple consecutive dilutions coupled with external mechanical treatment in accordance with homeopathic technology. After the dilution, the resulting carrier is impregnated onto solid neutral carrier. Etiology of speech disorders is described for example in https://en.wikipedia.org/wiki/Speech disorder, which is incorporated herein by reference. Anxiety of any etiology may be treated. Any number of dilutions maybe used in this method of the invention, however C200 dilution is preferred. Preferably, the pharmaceutical composition is prepared by using brain biopotentials of five healthy donors. Preferably, the patient is a child.

In accordance with another aspect, the present invention provides a method of treating autism in a human patient by administering an effective amount of the pharmaceutical composition in accordance with an embodiment of the present invention to the patient in need thereof. Preferably, after energy treatment of the initial neutral carrier, the resulting initial carrier is subjected to multiple consecutive dilutions coupled with external mechanical treatment in accordance with homeopathic technology. After the dilution, the resulting carrier is impregnated onto solid neutral carrier. Etiology of autism is described for example in https://en.wikipedia.org/wiki/Autism, which is incorporated herein by reference. Any number of dilutions maybe used in this method of the invention, however C200 dilution is preferred. Preferably, the pharmaceutical composition is prepared by using brain biopotentials of five healthy donors. Preferably, the patient is a child.

In accordance with another aspect, the present invention provides a method of treating tension headaches in a human patient by administering an effective amount of the pharmaceutical composition in accordance with an embodiment of the present invention to the patient in need thereof. Preferably, after energy treatment of the initial neutral carrier, the resulting initial carrier is subjected to multiple consecutive dilutions coupled with external mechanical treatment in accordance with homeopathic technology. After the dilution, the resulting carrier is impregnated onto solid neutral carrier. Etiology of tension headaches is described for example in https://en.wikipedia.org/wiki/Tension headache, which is incorporated herein by reference. Any number of dilutions maybe used in this method of the invention, however C200 dilution is preferred. Preferably, the pharmaceutical composition is prepared by using brain biopotentials of five healthy donors.

In accordance with another aspect, the present invention provides a method of treating pharmacoresistent epilepsy in a human patient by administering an effective amount of the pharmaceutical composition in accordance with an embodiment of the present invention to the patient in need thereof. Preferably, after energy treatment of the initial neutral carrier, the resulting initial carrier is subjected to multiple consecutive dilutions coupled with external mechanical treatment in accordance with homeopathic technology. After the dilution, the resulting carrier is impregnated onto solid neutral carrier. Etiology of epilepsy is described for example in https://en.wikipedia.org/wiki/Epilepsy, which is incorporated herein by reference. Any number of dilutions maybe used in this method of the invention, however C200 dilution is preferred. Preferably, the pharmaceutical composition is prepared by using brain biopotentials of five healthy donors.

In accordance with another aspect, the present invention provides a method of treating dementia in a human patient by administering an effective amount of the pharmaceutical composition in accordance with an embodiment of the present invention to the patient in need thereof. Preferably, after energy treatment of the initial neutral carrier, the resulting initial carrier is subjected to multiple consecutive dilutions coupled with external mechanical treatment in accordance with homeopathic technology. After the dilution, the resulting carrier is impregnated onto solid neutral carrier. Etiology of dementia is described for example in https://en.wikipedia.org/wiki/Dementia, which is incorporated herein by reference. Any number of dilutions maybe used in this method of the invention, however C200 dilution is preferred. Preferably, the pharmaceutical composition is prepared by using brain biopotentials of five healthy donors.

In accordance with another aspect, the present invention provides a method of improving mental and emotional wellbeing of an animal and/or reducing stress exerted on an animal by administering an effective amount of the pharmaceutical composition in accordance with an embodiment of the present invention to said animal. Preferably, after energy treatment of the initial neutral carrier, the resulting initial carrier is subjected to multiple consecutive dilutions coupled with external mechanical treatment in accordance with homeopathic technology. Any number of dilutions maybe used in this method of the invention, however C200 dilution is preferred. Preferably, the pharmaceutical composition is prepared by using brain biopotentials of five healthy donors.

### BRIEF DESCRIPTION OF THE DRAWINGS

FIG. 1 shows a flow diagram illustrating the device for producing the energy treated carrier.

FIG. 2 shows the device having several active electrodes and two current electrical electrodes.

FIG. 3 shows the device having one common reference electrode, veral active electrodes and several electrodes for passing current.

FIG. 4 shows the device having a digital data recording unit.

FIG. 5 shows that the negative symptoms were significantly reduced following 6 hours after the initial doses of test drug.

FIG. 6 shows the differences in patients' health status between baseline and 6 hours after the initiation of AP.

FIG. 7 shows the drug's efficacy in children with different developmental speech and language disorders.

FIG. 8 shows the drug's efficacy in children with different developmental speech and language disorders.

FIG. 9 shows the analysis of the dynamics of changes on different domians.

FIG. 10 shows the reduction of behavioral and pathological symptoms of dementia.

FIG. 11 shows cumulative grades of emotional-psychological status of dogs after administration of the AP drug and placebo.

### DESCRIPTION OF PREFERRED EMBODIMENTS

In one preferred embodiment, the energy treated carrier is used in a liquid form,

wherein the liquid form of the energy treated carrier is prepared as a mixture of several homeopathic dilutions;

wherein the liquid form of the energy treated carrier is prepared as a mixture of several centesimal homeopathic dilutions.

In other embodiments, the drug may be prepared in a solid dosage form containing an effective amount of a solid neutral carrier impregnated with the liquid form of the energy treated carrier, and pharmaceutically acceptable excipients.

Preferably, said energy-treated carrier is activated (potentized) by repeated serial dilution in an aqueous or aqueous-alcoholic diluent in combination with external mechanical impact—shaking of each resulting dilution.

Alternatively, the energy exposed neutral carrier is activated (potentized) by repeated serial grinding—trituration with a solid carrier according to homeopathic methodology.

The initial carrier may be distilled or purified (special purification grade) water or water-alcoholic solution with a maximum conductivity of 0.1 mS/m at 25° C., or an activated (potentized) carrier containing distilled or purified (special purification grade) water or water-alcoholic solution with a maximum conductivity of 0.1 mS/m at 25° C., which has been treated by repeated serial dilution in an aqueous or aqueous-alcoholic diluent in combination with external mechanical impact.

Alternatively, the energy exposure of the initial carrier may be accomplished by exposing it to an electrical current caused by differences of potentials proportional in amplitude to the difference of biopotentials of referential derivations which are applied to a set of electrodes placed in the initial carrier, one of which is fed potentials from the reference ear-lobe electrode and the others receive amplified potentials from the active electrodes positioned at different scalp locations.

Alternatively, the difference of potentials being applied to the pair of electrodes in the initial carrier is proportional in amplitude to the difference of biopotentials recorded in a bipolar derivation consisting of active recording electrodes placed at different locations on the scalp.

Alternatively, the difference of potentials being applied to the pair of electrodes in the initial carrier is proportional in amplitude to the difference of biopotentials recorded in a referential derivation consisting of an active scalp electrode and a reference electrode located on the ear-lobe.

Alternatively, the difference of potentials being applied to at least two electrodes in the initial carrier is proportional in amplitude to an amplified summed signal formed by adding up the differences of bioelectric potentia s from referential derivations of recording electrodes that contribute to different channels from different scalp locations.

Alternatively, the difference of potentials being applied to at least two electrodes in the initial carrier is proportional in amplitude to an amplified averaged signal formed by adding up the differences of bioelectric potentials from referential derivations of recording electrodes that contribute to different channels from different scalp locations.

Alternatively, the difference of potentials being applied to at least two electrodes in the initial carrier is proportional in amplitude to an amplified summed or averaged signal formed by adding up the differences of bioelectric potentials from referential derivations of recording electrodes that contribute to different channels from different locations on the scalp of a patient.

Alternatively, the difference of potentials being applied to at least two electrodes in the initial carrier is proportional in amplitude to an amplified summed signal formed by adding up the differences of bioelectric potentials from referential derivations of recording electrodes that contribute to different channels from different locations on the scalp of a donor.

Alternatively, the difference of potentials being applied to at least two electrodes in the initial carrier is proportional in amplitude to a combined summed difference formed by adding up the summed signals (biopotential differences) recorded in individual referential derivations from different locations on the scalp of two or more donors, preferably five donors.

Alternatively, the pharmaceutical composition is prepared as a mixture of at least two ingredients, one of which is prepared by exposing the carrier to the energy treatment using the difference of potentials applied to electrodes in the initial carrier being proportional in amplitude to the difference of biopotentials of electrodes recording from different sites of the brain of a patient, and the other ingredient is obtained by exposing the carrier to the energy treatment using the difference of potentials applied to electrodes in the initial carrier being proportional in amplitude to the difference of biopotentials of electrodes recording from different sites of the brain of one or more donors.

Also, the pharmaceutical composition may be prepared as a mixture of at least two ingredients, each of which is obtained by exposing the carrier to the energy treatment using the difference of potentials applied to electrodes in the initial carrier, the difference being proportional in amplitude to the biopotential difference of electrodes recording from different sites of the brain of donors.

Preferably, the differences of biopotential are initially recorded in referential derivations by recording electrodes that contribute to separate channels from different locations on the scalp and are referenced to a zero potential point (underlying the reference electrode) on the ear-lobe, following which they are amplified separately in each channel, the amplified outputs being then digitized and the resulting digitized and digitally filed signals are transferred into the memory of a programmable digital signal summing unit—a signal summer, where the digital signals are added up to produce a summed digital signal representing the overall (summed) potential difference of the referential derivations employed, which is thereafter inputted to a digital-to-analog converter of an arbitrary waveform generator, where it is converted back to a summed analog signal to generate output signals in the form of voltages (potential difference) which approximate the waveform of the summed difference of bioelectric potentials recorded, and are fed onto at least two current electrodes immersed in the container with the initial neutral carrier.

Preferably, the initially amplified signals are filtered by means of a low-pass filter with cut-offs of 0.5÷35 Hz.

The output difference of potentials that approximates the waveform of the resultant (total) difference of the bioelectric potentials recorded and is fed into the current electrodes in the container with the initial carrier has a peak amplitude of 0.5÷1.5 volts.

As an alternative, the differences of biopotential are initially recorded in referential derivations by recording electrodes that contribute to separate channels from different locations on the scalp and are referenced to a zero potential point (underlying the reference electrode) on the ear-lobe, following which they are amplified separately in each channel, the amplified outputs being then digitized and resulting digitized and digitally filed are entered into the memory of a programmable data recording unit and recorded onto an electronic data storage device (data storage medium), whence the data is transferred into the memory of a programmable digital signal summing unit—a signal summer, where the digital signals are added up to produce a summed digital signal representing the overall (summed) potential difference of the referential derivations employed, which is thereafter input to a digital-to-analog converter of an arbitrary waveform generator, where it is converted back to a summed analog signal to generate output signals in the form of voltages (potential difference) which approximate the waveform of the summed difference of bioelectric potentials recorded, and are fed onto at least two current electrodes immersed in the container with the initial neutral carrier.

Preferably, the initially amplified signals are filtered by means of a low-pass filter with cut-offs of 0.5÷35 Hz.

In one variant, initially, individual differences of biopotentials are independently recorded in individual referential derivations of recording electrodes that contribute to separate channels from different locations on the scalp and are referenced to a zero potential point (underlying the reference electrode) on the ear-lobe of different subjects, following which the individual potential differences recorded in each subject's referential derivations are independently amplified in each separate channel, the amplified outputs being then digitized and the individual signals digitized and digitally filed signals of each subject's potential differences entered into the memory of a programmable data recording unit and recorded onto individual electronic data storage devices, whence the individually filed data is transferred into the memory of a programmable digital signal summing unit—signal summer, where the digital signals of individual biopotential differences are added up to produce a combined summed digital signal representing the overall summed potential difference of individual referential derivations employed, which is thereafter input to a digital-to-analog converter of an arbitrary waveform generator, where it is converted back to a resultant analog signal to generate output signals in the form of voltages (potential difference) which approximate the waveform of the combined summed difference of bioelectric potentials recorded in individual referential derivations of several subjects and are fed onto at least two current electrodes immersed in the container with the initial neutral carrier.

Preferably, the initially amplified signals are filtered by means of a low-pass filter with cut-offs of 0.5÷35 Hz.

In one aspect, the invention provides a device, which is used to produce the claimed pharmaceutical composition for treating nervous and mental diseases, comprising at least two recording electrodes that pick up bioelectric potentials from different brain sites, and are transformed into the inputs of a channel amplifier of an EEG recording unit, with the outputs connected to at least two current electrodes of an energy treatment unit which are placed in the container with the initial liquid carrier.

Preferably, the device includes recording electrodes, one of which is located on the ear-lobe, serving as a reference, and the other active electrodes are attached to the scalp surface at locations overlying different brain sites, wherein the reference and one active electrode are connected to the inputs of each channel amplifier of the EEG unit, with the outputs of all channel amplifiers connected in parallel to two elewctrical electrodes of the energy treatment unit which are placed in the container with the initial liquid carrier

In one variant, the device includes recording electrodes, one of which is located on the ear-lobe, serving as a reference, and the other active electrodes are attached to the scalp surface at locations overlying different brain sites, wherein the reference and one active electrode of each of the referential derivations are connected to the inputs of each channel amplifier of the EEG unit, with the reference outputs of all channel amplifiers connected to a common current electrode and the active outputs linked to the current electrodes of the energy treatment unit, the number of which is equivalent to the number of the active electrodes employed.

In one variant, the device includes an EEG unit in which the electrodes are a reference electrode to be attached to the ear-lobe and one or more active electrodes that pick up bioelectric potentials from different brain sites—at relevant scalp locations and make up referential derivations contributing to the inputs of channel amplifiers, each with the outputs connected to the inputs of a respective analog-to-digital converter, the digital signal outputs of which are linked to the inputs of a programmable digital data recorder used to feed the data into electronic storage devices; and a data processing unit which comprises a programmable digital signal summing unit—signal summer, the inputs of which are fed the digital outputs from the respective analog-to-digital converter of the EEG unit and/or the individually filed data from the electronic storage devices, with the outputs connected to the inputs of a digital-to-analog converter of an arbitrary waveform generator which generates output signals in the form of voltages (potential difference) that approximate the waveform of the summed potential difference of all biopotential recordings employed, and the analog outputs of which are attached to at least two current electrodes of the energy treatment unit which are immersed in the container with the initial liquid carrier.

Preferably, the device further includes s a low-pass filter with cut-offs of 0.5÷35 Hz connected between the channel amplifier and the analog-to-digital converter.

While the invention is not limited to any specific theory, it has been surprisingly discovered by the inventors that neutral carrier treated with energy proportional to bioelectric potentials of a human brain or nervous tissue has efficacy in treating a variety of nervous and mental conditions and deceases. The biological activity of the neutral carrier is especially distinct when the initial liquid carrier is subjected to multiple consecutive dilutions coupled with external shaking of each dilution in accordance with homeopathic technology. While the invention is not limited to any specific theory, it is contemplated that pathogenesis of deceases and conditions treatable with the pharmaceutical compositions of the invention can be attributed to abnormal changes in features and parameters of brain bioelectrical activity, wherein the additional activation of the energy-treated carrier by repeated serial dilution in an aqueous or aqueous-alcoholic diluent in combination with external mechanical impact—shaking of each resulting dilution, preferably based on homeopathic methodology enhances the drug's therapeutic efficacy and also reduces potential toxicity, making its use safer, which is supported by numerous research studies.

Furthermore, the presence in the claimed device of programmable digital signal summing unit—signal summer, designed as a microcomputer, which generates, based on a preset program, a summed or averaged signal that approximates the waveform of the total biopotential difference made up of all potential differences from the various set-ups of referential and/or bipolar derivations from one or several individuals, which have been recorded onto electronic storage devices, extends the functional, therapeutic potential of the claimed drug.

The claimed pharmaceutical composition, both as a non-activated energy-treated carrier and activated (potentized) form prepared by multiple consecutive dilution in an aqueous or aqueous-alcoholic alcoholic diluent in combination with external mechanical impact—shaking of each resulting dilution, adds to the range of agents useful in treating diseases and disorders of the nervous system.

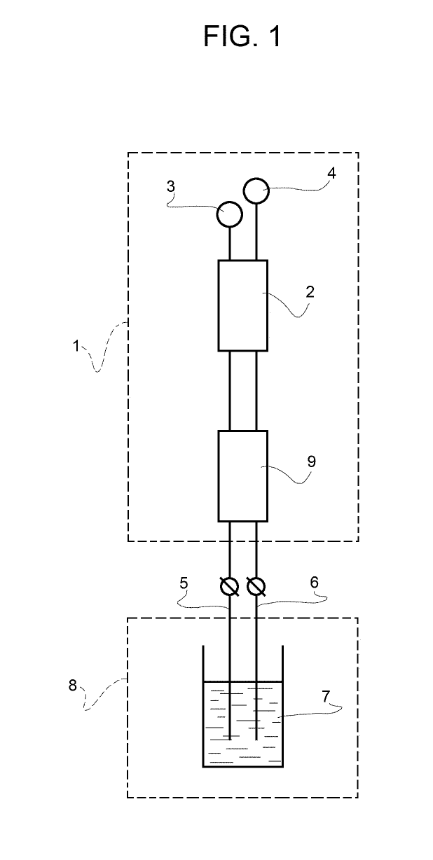

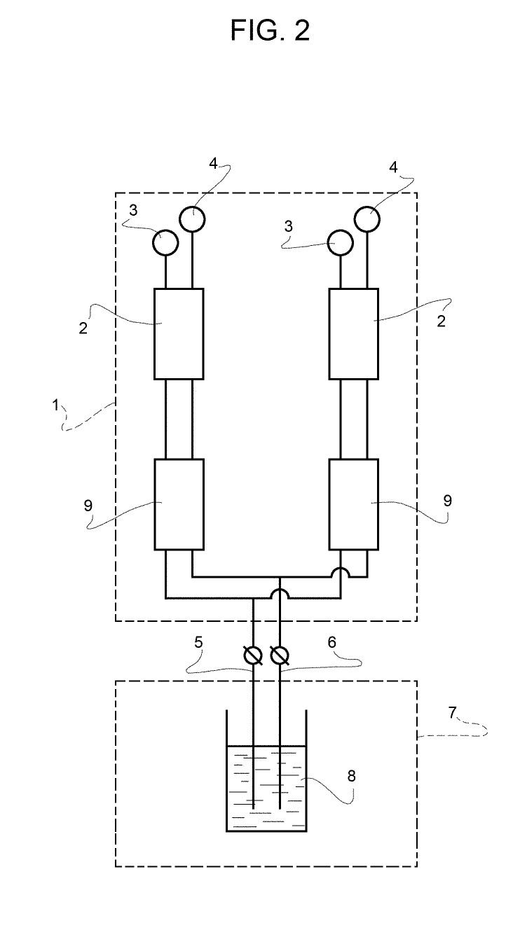

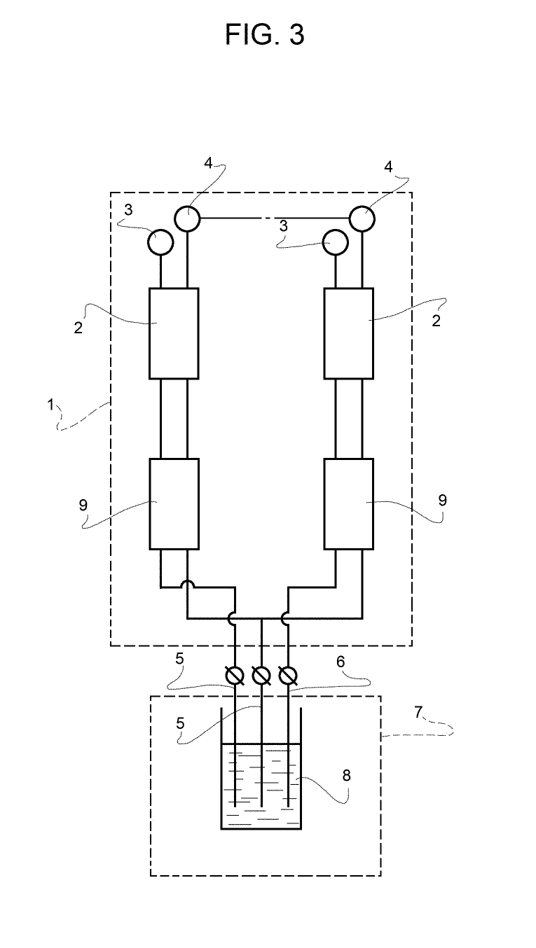

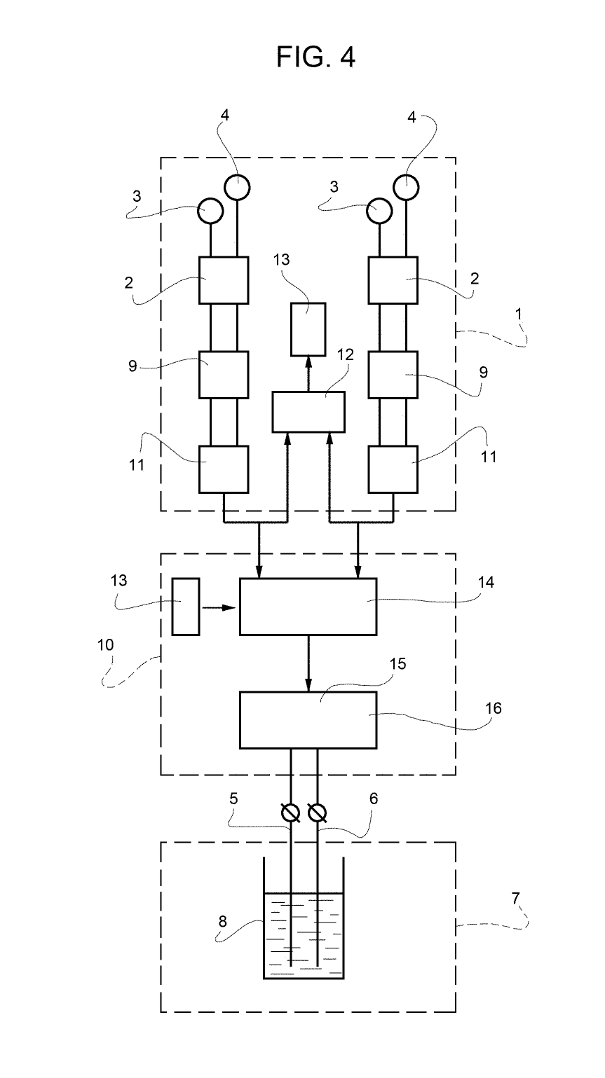

FIG. 1 shows a flow diagram that illustrates an embodiment of the device for producing the energy treated carrier, which contains a pair of recording electrodes and a pair of current electrodes; FIG. 2 presents an alternative flow diagram of the device which contains several active electrodes for reading biopotentials of the brain and two current electrical electrodes for passing electric current through the initial liquid carrier; FIG. 3 shows an alternative embodiment of the device, which contains one common reference electrode, several active electrodes for recording brain biopotentials, and several electrodes for passing current through the initial carrier; FIG. 4 shows an alternative embodiment of the device which contains a digital data recording unit.

The device of this aspect of the invention (FIG. 1) includes EEG recording unit 1, which incorporates at least one channel amplifier 2, the inputs of which are connected to recording electrodes 3 and 4 which pick up bioelectric potentials from different brain sites—at relevant locations on the scalp, and which may be available as metal discs or rods of various shapes (cup-shaped, flat, or needle electrodes), with the diameter of the contact area of 0.4÷1.0 cm, held in place on the head with gels, a head mesh, or a silicone cap (cup-shaped silver-chlorine electrodes are recommended by international standards), wherein active recording electrode 3, placed on the scalp (over the brain), is connected to the first input of channel amplifier 2 whereas reference (zero) recording electrode 4, located at some distance from the brain (normally on the ear-lobe), is inserted into the second input of the channel amplifier; or two active scalp electrodes 3 (not shown in the figure) that are connected to both inputs of channel amplifier 2. The outputs of the channel amplifier 2 are attached, respectively, to two electrodes 5 and 6 for passing electrical current through the initial liquid carrier of the energy treatment unit 7, which current electrodes are made from corrosion-resistant material, preferably stainless steel, the current electrodes may be placed in the container 8 with the initial liquid carrier. The initial liquid carrier is distilled or purified (special purification grade) water or water-alcoholic solution with maximum conductivity of 0.1 mS/m at 25° C. The outputs of channel amplifier 2 may be linked to a low-pass filter 9 with cut-offs (bandpass) of 0.5 to 35 Hz.

Alternatively, the device shown in FIG. 2 contains a set of channel amplifiers 2, the first input of each amplifier being fed from active recording electrodes 3 (placed at different scalp locations overlying the brain) and reference (zero) electrode 4 inserted into the second input of each of the amplifiers. The outputs of all channel amplifiers 2 are connected in parallel to two current electrodes 5 and 6 of energy treatment unit 7, which are placed in container 8 with the initial liquid carrier, thus enabling summing or averaging of the potential differences of referential derivations immediately inside container 8. The outputs of channel amplifiers 2 may be linked to a low-pass filter 9 with cut-offs (bandpass) of 0.5 to 35 Hz.

Alternatively, the device shown in FIG. 3 may contain a number of channel amplifiers 2, with the first input of each linked to one of active recording electrodes 3 (placed at different scalp locations overlying the brain) and the second connected to reference (zero) recording electrode 4. The active outputs of said channel amplifiers 2 are separately (or in groups) attached to different current electrodes 5 of energy treatment unit 7, and short-circuited (joined together) zero outputs of channel amplifiers 2 are connected to zero current electrode 6, providing the summing and averaging of biopotential differences in the referential derivations straight in container 8. The outputs of channel amplifiers 2 may be connected to low-pass filter 9 with the cut-offs (bandpass) of 0.5 to 35 Hz.

Alternatively, the device shown in FIG. 4 includes EEG recording unit 1, data processing unit 10 and energy treatment unit 7. The EEG recording unit 1 contains a set of channel amplifiers 2, with the first input of each attached to one of active recording electrodes 3 (involved in referential derivations from different scalp locations over the brain) and the second connected to reference (zero) recording electrode 4. The outputs of each channel amplifier 2 are separately connected to the inputs of respective analog-to-digital converters 11, whose digital signal outputs are connected to the inputs of programmable data recording unit 12 used to record information onto storage device 13. Data processing unit 10 comprises programmable digital signal summing unit 14—signal summer, designed as a microcomputer, which is input the recorded digital data from the outputs of respective analog-to-digital converters 11 of EEG recording unit 1 and/or the digital data recorded as individual files on the electronic storage devices, its outputs being attached to the inputs of digital-to-analog converter 15 of arbitrary waveform generator 16 which generates output signals in the form of voltages (potential difference) which approximate the waveform of the summed biopotential difference of all recordings performed. The analog outputs of arbitrary waveform generator 16, with the peak amplitude of the signals yielded of up to 5-10 volts, are connected to at least two current electrodes 5 and 6 of energy treatment unit 7, which are placed in container 8 with the initial neutral carrier. The outputs of channel amplifiers 2 may be connected to low-pass filter 9 with the cut-offs (bandpass) of 0.5 to 35 Hz.

Alternatively, the energy-treatment of the initial carrier may be accomplished by passing through it an electrical current caused by a difference of electrical potentials proportional in amplitude to the amplified difference of biopotentials summed over all nervous tissue or brain recordings performed at a distance by ultrasensitive magnetometers—superconducting quantum interference devices—immersed in liquid helium (see A. N. Shestakova, A. V. Butorina, A. E. Osadchy, Y. Y. Shtyrov. ‘Magnetoencephalography—a novel functional brain mapping technique for use in humans’. Experimentalnaya Psikhologia [Experimental psychology] (in Russian), 2012, vol. 5, iss. 2, pp. 119-134).

The pharmaceutical composition of the invention may be prepared as follows.

Initially, a scalp EEG is taken from a subject (patient or donor) who is placed during the measurements in a light- and sound-proof room and seated in a comfortable chair, with the eyes closed.

In one valiant, at least two electrodes of EEG recording unit 1 (FIG. 1) are attached to different locations on the scalp of the subject to be exposed to the therapeutic effect of the drug, wherein the electrodes record brain's electrical activity by means of referential derivation and input the acquired bioelctric potentials to channel amplifier 2. The referential derivation consisting of active scalp electrode 3 and reference electrode 4, positioned, most commonly, on the ear-lobe; or bioelectric potentials are input to channel amplifier 2 as a bipolar derivation consisting of active recording electrodes 3 placed at different scalp locations (not shown in the figure). The amplified potentials (the peak amplitude of the output signal being in the range of 0.5÷1.5 volts) are run through low-pass filter 9 with cut-offs (passband) of 0.5 to 35 Hz and fed into two current electrodes 5 and 6 of energy treatment unit 7, which are placed in container 8 with the initial carrier, thus providing primary energy treatment of said carrier by passing through it an electrical current caused by a potential difference proportional in amplitude to the bioelectric potential difference of the brain recordings. The initial (starting) carrier is preferably distilled or purified (special purification grade) water or water-alcoholic solution with a maximum conductivity of 0.1 mS/m at 25° C.

Said energy treatment of the initial neutral carrier is carried on, preferably, for 5÷10 minutes.

The substance prepared in the above process (liquid energy-treated carrier), depending on the particular location of active scalp electrode 3 and placement of reference electrode 4, may be used as a standalone drug in individualized therapy for a particular disease or disorder of the nervous system of the subject (patient) whose individual features the drug is directed at, or as a matrix solution for further activation (potentization) procedure, i.e. repeated serial dilution in an aqueous or aqueous-alcoholic diluent in combination with external mechanical impact (shaking at each dilution step).

To prepare a pharmaceutical composition with broader therapeutic potential, the energy-treatment of the initial liquid carrier may be carried out by passing through it an electrical current caused by a difference of potentials proportional in amplitude to the difference of biopotentials summed or averaged over a series of bipolar and/or referential derivations, used in various set-ups, from different brain sites—locations on the scalp of a patient, of the individual to be treated or a different (healthy) individual—a donor. The EEG may be recorded by placing on the subject's head a silicone or textile cap with attached electrodes that pick up electrical activity at different locations on the head, which, for example, as determined by the internationally recognized clinical 10-20 system, involves 19 recording electrodes 3 placed in regular positions all over the head, and one reference electrode 4 located at a distance from the brain—most commonly on the ear-lobe (or a pair of short-circuited reference electrodes 4 attached to both ear-lobes).

Active recording electrode 3, placed at distinct scalp locations (overlying the brain), are inserted into the first input of each respective channel amplifier 2 (FIG. 2), with the second input of each amplifier being connected to reference (zero) recording electrode 4, located, most commonly, on the ear-lobe (off the brain), wherein the amplified potentials output by each channel amplifier 2 (the peak amplitude of the output signal being in the range of 0.5÷1.5 volts) are run through low-pass filter 9 with cut-offs (passband) of 0.5 to 35 Hz and fed in parallel into two electrical electrodes 5 and 6 for passing electric current of the energy treatment unit 7, which are placed in container 8 with the initial liquid carrier, thus enabling the amplified potential differences obtained in referential derivations to be summed or averaged immediately inside container 8.

Alternatively, (FIG. 3) active recording electrodes 3 are inserted into the first input of each respective channel amplifier 2, the second input of each amplifier being connected to reference (zero) recording electrode 4. The amplified potentials at the active outputs of all channel amplifiers 2 are separately (or in sets) run through low-pass filter 9 with cut-offs (passband) of 0.5 to 35 Hz and fed into different current electrodes 5 of energy treatment unit 7 whereas zero current electrode 6 is fed potentials from the short-circuited (joined together) zero outputs of channel amplifiers 2, thus enabling the potential differences obtained in referential derivations to be summed or averaged immediately inside container 8.

The energy treatment of the initial liquid carrier is carried out, preferably, for 5÷10 minutes.

The outputs of channel amplifiers 2 may be connected to low-pass filter 9 with the cut-offs (bandpass) of 0.5 to 35 Hz.

In another embodiment, the claimed method for producing the drug with a broad therapeutic potential in treating diseases and disorders of the nervous system may be implemented as follows.

In the first step, individual EEGs are independently recorded by means of referential derivations as individual differences of potentials from different locations on the scalp of different, preferably healthy, donors.

For this purpose, the subject (donor—healthy individual) is normally put on a silicone or textile head cap with attached electrodes that record, in referential derivations, the electrical activity at different locations on the subject's head and input the signals into multichannel amplifiers 2 of EEG recording unit 1 (FIG. 4). Preferably, based on the internationally recognized clinical 10-20 system, this involves 19 recording electrodes 3 placed in regular positions all over the head, and one reference electrode 4 located at a distance from the brain—most commonly on the ear-lobe (or two short-circuited reference electrodes 4 attached to both ear-lobes). Further, from active recording electrodes 3 involved in referential derivations from distinct scalp locations (overlying the brain), the biopotential are fed to the first input of each channel amplifier, while the second input of each amplifier is connected to reference (zero) recording electrode 4. The amplified potential outputs from each channel of each channel amplifier 2 are separately filtered by low-pass filter 9 with cut-offs (passband) of 0.5 to 35 Hz and fed to the inputs of respective analog-to-digital converters 11, where the analog signals are converted into digital data, following which the individual digitized and filed potential difference signals are transferred to the memory of programmable recording unit 12 used to record data onto electronic data storage devices 13. Individual EEGs are recorded for 5÷10 minutes.

In the next step, the individual data recorded on electronic storage devices 13 and/or digital output signals from analog-to-digital converters 11 are entered into the memory of programmable digital signal summing unit 14 (a minicomputer-based signal summer) of data processing unit 10 and added up using a preset program which allows for generating output signals as a combination of several individual digital files containing individual potential differences from EEGs (or EEG fragments) recorded independently in different subjects. From programmable digital signal summing unit 14—signal summer, the combined summed output signal representing the overall summed potential difference of individual referential derivations employed is input to digital-to-analog converter 15 of arbitrary waveform generator 16, where it is converted into a resultant analog signal to generate the output signal in the form of voltages (potential difference) which approximate the waveform of the combined summed biopotential difference of all individual referential derivations from the bodies of several donors, and which are fed to at least two current electrodes 5 and 6 of energy treatment unit 7, placed in container 8 with the initial carrier.

Alternatively, the energy treatment of the neutral carrier, placed in container 8 of energy treatment unit 7, may be accomplished by means of an electrostrictive element or membrane of an electro-acoustic transducer immersed in the initial carrier or, for example, using an electromagnetic coil (not shown in the figure), onto which the control output signal from arbitrary waveform generator 16 is fed in the form of voltages (potential difference) which approximate the waveform of the combined summed biopotential difference of all recordings carried out.

The activated (potentized) form of the energy-treated carrier used in the claimed drug is prepared by uniformly reducing the concentration of above matrix solution, prepared previously as a liquid energy-treated carrier, via serial dilution of 1 volumetric part of solution to be processed, beginning with the matrix solution, in 9 volumetric parts (for decimal D dilution—on the homeopathic scale) or 99 volumetric parts (for centesimal C dilution), or 999 volumetric parts (for M dilution) of vehicle, combined with repeated succussion (potentization, or ‘dinamisation’) of each resulting dilution and using a single or separate containers for each subsequent dilution—up to the target potency (i.e. degree of dilution, as defined in homeopathic art; e.g., see W. Schwabe, ‘Homeopathic medicines’ (in Russian), Moscow, 1967, pp. 14-29 or G. Koehler, ‘Homeopathic medicine’ (in Russian), Moscow, Meditsina, 2000, part 1, pp. 37-40; RU 2500426; U.S. Pat. No. 7,229,648; U.S. Pat. No. 4,311,897). In the first step, the homeopathic dilution process is carried out using a vehicle, which is preferably a 10÷15% ethanol solution (in water) or distilled water, whereas further dilutions are obtained with the use of a 20÷30% solution of ethanol in water, and the final (two or three last dilutions) dilution steps are performed using a 30÷75% ethanol solution (in water) or distilled water.

The exposure to external impact through the concentration reduction process can also be attained by using ultrasound, electromagnetic, or physical factors.

For example, in order to prepare the 12th centesimal C12 dilution, one volumetric part of the matrix solution is diluted in 99 volumetric parts of a neutral, aqueous (or aqueous-alcoholic) diluent (vehicle) and repeatedly (at least 10 times) shaken (potentized), preferably vertically, to obtain the 1st centesimal C1 dilution. Said 1st centesimal C1 dilution is used to prepare the 2nd centesimal C2 dilution. This procedure is repeated 11 times to obtain the 12th centesimal C12 dilution. Thus, the 12th centesimal C12 dilution is a solution yielded by serial (12 times) dilution of 1 volumetric part of an initial matrix solution in 99 volumetric parts of a vehicle, using a different container each time, which is a solution obtained by diluting the matrix 100¹² times. Similar procedures, with relevant degrees of dilution, are utilized to obtain other dilutions, such as C30, C50, C200 etc. It is preferable to prepare the activated (potentized) form of the drug as a C200 dilution. However, any individual homeopathic dilution or mixture of dilutions can be used and is contemplated. It is to be determined experimentally whether a particular dilution will sufficiently potentiate the initial energy signature imprinted on the initial liquid carrier. Since not potentiated liquid carrier also has activity, the entire range of dilution is within the scope of the invention.

For example, when preparing the activated (potentized) form of the drug as a mixture of different (predominantly centesimal) dilutions, each dilution (e.g., C12, C30, and C50) should be produced separately using the above process, which is carried on in each case until a target potency reduced by three 3 dilution steps is obtained (until dilutions C9, C27, C47, respectively) and then transferred, together with the other dilutions employed in the formulation—one volumetric part of each preparation, to a single container and mixed with the required volume of diluent (97 parts for a centesimal dilution, respectively) Next, the resulting mixture is consecutively diluted 1:100 two more times, each followed by a potentization step. This yields an activated (potentized) form of the energy-treated carrier, which is used to produce the claimed ultra-low dose drug—the form resulting from the matrix solution diluted 100¹², 100³⁰ and 100⁵⁰ times, i.e. C12, C30 and C50 dilutions, respectively.

The activated (potentized) form of the drug may be a mixture of other dilution combinations including, for example, decimal and/or centesimal dilutions (D20, C30, C100; D10, C12, C100; or C12, C30, C200 etc.) prepared using homeopathic methodology, and whose efficacy is determined experimentally.

When in mixtures (for example, C12, C30 and C50), the dilutions should be mixed, preferably, in 1:1:1 ratio (v/v), and used in the liquid dosage form.

The claimed drug may be produced in the solid form (e.g. powder, granules or tablets) which contains a specified (effective) amount of neutral carrier (such as lactose) impregnated by soaking it to saturation in the liquid activated (potentized) form of the energy-treated carrier, and pharmaceutically acceptable excipients, preferably including lactose, microcrystalline cellulose, and magnesium stearate.

The solid tablet form is provided by an irrigation process on a fluidized bed system (such as Hüttlin Pilotlab by Hüttlin GmbH), involving soaking to saturation of a powdered (particle size, 50÷500 μm) neutral matter, e.g. lactose (milk sugar), which is fed into the fluidized boiling bed containing the previously prepared aqueous or aqueous-alcoholic solution of liquid energy-treated carrier in the activated (potentized) form, preferably in the ratio of 1 kg of solution to 5 or 10 kg of lactose (1:5-1:10), followed by drying via heated air supplied into the process chamber. An predefined amount of lactose (10÷91% of total tableting blend) impregnated with the activated (potentized) form of energy-treated carrier as described above is loaded into a blender and processed together with lactose pre-wetted with the activated (potentized) of energy-treated carrier (3÷10% of tableting blend), and plain lactose taken in the amount of 84% of total tableting blend (for the purpose of cost reduction and a more simplified and faster process, with the strength of therapeutic effect preserved). Thereafter microcrystalline cellulose (5÷10% of total tableting blend) and magnesium stearate (1% of total tableting blend) are added to the ingredients mixed in the previous step. The resulting blend is uniformly mixed and subjected to dry compression (e.g., on a tablet press), during which 150÷500 mg tablets are produced. The compression step yields tablets containing lactose impregnated with water-alcohol solution of the activated (potentized) form of energy-treated carrier, produced from the matrix solution diluted, for example, 100²⁰⁰ times (equivalent to centesimal C200 dilution) or, for example, 100¹², 100³⁰ and 100⁵⁰ times, which corresponds to a mixture of centesimal C12, C30 and C50 dilutions prepared according to homeopathic methodology.

Alternatively, the activated (potentized) form of energy-treated carrier of the claimed drug may be prepared as a triturated powder (trituration) by uniform ‘dry dilution’ of said matrix solution, previously prepared as a liquid energy-treated carrier, which involves serial fine grinding of 1 weight part of each trituration, beginning with the matrix, with 9 weight parts of (for decimal homeopathic scale, D) or 99 weight parts of (for centesimal homeopathic scale, C), or 999 weight parts (for millesimal homeopathic dilution, M) of neutral carrier (preferably lactose (milk sugar) granules) with the use of separate containers for each successive trituration until the target potency, i.e. degree of trituration on the homeopathic scale (e.g., see W. Schwabe, ‘Homeopathic medicines’ (in Russian), Moscow, 1967, pp. 20-29).

Below are case examples of treating diseases or disorders of the nervous system.

### Example 1

In a randomized, double-blind, placebo-controlled clinical trial, the claimed drug, formulated as a solid dosage form containing lactose granules impregnated with water-alcohol solution of the liquid activated (potentized) energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a potential difference being applied to electrodes placed in the initial neutral carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug—was examined for efficacy and safety in treating abnormal craving for alcohol (ACA) in alcohol abstinent patients (in remission).

The purpose of the study was:

1. To evaluate the clinical efficacy and safety of AP drug in a medium-term relapse prevention treatment in alcohol dependent patients;
2. To investigate the effect of AP drug on the occurrence of relapses in alcohol dependence;
3. To investigate the effect of AP drug on primary craving for alcohol in patients in remission.
4. To examine the effects of AP drug on psychopathological, autonomic and somatic disturbances in patients after withdrawal, in the stage of developing remission.
4. To evaluate the sustainability of therapeutic effect of AP drug at 4 weeks after the last dose of test drug.

Study design: a randomized, double-blind placebo-controlled study in parallel groups: 30 patients in group 1 (AP drug) and 30 in group 2 (placebo). Participation period: 28 weeks (treatment—24 weeks, follow up—4 weeks).

The study recruited outpatients (age range, 20 to 55 years) diagnosed with moderate alcohol dependence syndrome in accordance with ICD-10 (F10.222). A baseline screening was performed which involved recording of medical history data, physical exam, severity assessment of clinical psychopathological, neurological, somatic and autonomic disorders, and laboratory testing.

At scheduled visits, the patients underwent health changes assessment (scoring on clinical scales to rate the severity of psychopathological, autonomic, somatic and neurological disorders).

Inclusion Criteria:

1. Male outpatients aged between 20 and 55 years.
2. Patients meeting the ICD-10 criteria for alcohol dependence, moderate severity (F10.222);
A. Prior history of strong desire or sense of compulsion to take alcohol.
B. Impaired capacity to control alcohol-taking behavior.
C. Evidence of tolerance to the effects of alcohol.
D. Preoccupation with alcohol use.
E. Persistent alcohol use despite clear evidence of harmful consequences.
F. A physiological withdrawal state.
3. Patients capable of adequately assessing own state.

Exclusion Criteria.

1. Narcotic drug use.
2. Regular psychoactive drug use.

### 3. Encephalopathy

4. Psychotic state (with delirium, hallucinations).
5. Epileptiform syndrome
6. Severe somatic and infectious diseases that can influence the participation in the study programme.

The prescribed dose of test drug was: 2 tablets 6 times through the day of enrollment, followed by 1 tablet 4 times daily for the rest of the days—sublingually, not together with food. It was allowed to increase the dose (to 8-12 tablets daily) with renewed alcohol craving. The overall duration of treatment was 24 weeks (6 months). The choice of the dose and treatment duration was based on previous clinical studies of AP drug. Concomitant medications: the patients were allowed to use medications for co-existing medical conditions, except for psychoactive agents. The dosage and dosing frequency prescribed for these medicines was to be (if possible) maintained throughout the study period. The following medications were prohibited during the study: antidepressants, antipsychotics, nootropics, anticonvulsants, sedative hypnotics, narcotic and other strong-acting drugs. At baseline, all patients were diagnosed with prevalent psychopathological disorders, with lesser autonomic, somatic and neurological problems. The patients reported craving for alcohol, emotional lability and depressed mood.

Study Results:

Significant positive changes in efficacy parameters were observed immediately after the initiation of treatment with AP drug and onward throughout the study. By the end of treatment week 1, patients in the group of AP drug demonstrated a statistically significant improvement in all scores on an ACA Clinical Symptoms Scale (Table 1).

TABLE 1
ACA scores of patients treated with AP drug (n = 30) (M ± m).

| | | In the first week | | |
|---|---|---|---|---|
| Parameter | Baseline | of treatment | 12 weeks | 24 weeks |
| Mean psychopathological | 24.6 ± 1.5 | 10.6 ± 1.4*** | 5.9 ± 0.51*** | 2 ± 0.27*** |
| symptoms total score | | | | |
| Mean autonomic and somatic | 5.4 ± 0.29 | 2.9 ± 0.2*** | 1.3 ± 0.1*** | 0.5 ± 0.08*** |
| symptoms total score | | | | |
| Mean neurological symptoms | 3.7 ± 0.28 | 2.1 ± 0.28* | 0.7 ± 0.12*** | 0.3 ± 0.1*** |
| total score | | | | |

Note:
*p < 0.05 from the baseline level;
**p < 0.001 from the baseline level;
***p < 0.001 from the baseline leve

This trend was maintained until the end of treatment. During the first 4 weeks of AP treatment, most patients reported normalization of sleep and somatic status (resolution of headache, dizziness and fatigue); by week 12, the patients' mood was stabilized and abnormal alcohol craving attenuated. Somatic symptoms were only observed at week 24 in patients who did not adhere to the prohibition on alcohol during the treatment.

Similar trends were observed in the placebo group, with, however, substantially higher symptom scores as rated on the health assessment scale, compared to the AP group (Table 2). Improvement was the slowest for changes observed in pathopsycological disturbances. In most patients in the placebo group, emotional lability and depressed mood remained unresolved until the end of the treatment.

TABLE 2
ACA Scale scores of patients receiving placebo (n = 30) (M ± m).

| | | In the first | | |
|---|---|---|---|---|
| Parameter | Baseline | week of treatment | 12 weeks | 24 weeks |
| Mean psychopathological | 22.1 ± 1.39 | 18.7 ± 1.32* | 13.2 ± 1.62* | 11.4 ± 1.29* |
| symptoms total score | | | | |
| Mean autonomic and somatic | 4.5 ± 0.43 | 4.1 ± 0.3 | 3.9 ± 0.24** | 2.6 ± 0.22** |
| symptoms total score | | | | |
| Mean neurological symptoms | 3.3 ± 0.6 | 3.2 ± 0.21 | 1.3 ± 0.19* | 1.1 ± 0.13* |
| total score | | | | |

Note:
*p < 0.05 from the baseline level;
**p < 0.001 from the baseline level;
***p < 0.001 from the baseline level

The analysis of disease relapses indicated the effect of AP drug on patients' capacity to abstain from alcohol consumption (Table 3).

TABLE 3
Relapse estimation outcomes in patient groups receiving AP drug
(n = 30) and placebo (n = 30) over the 28-week study period.

| Parameter | AP | PLACEBO |
|---|---|---|
| Single instance of alcohol use (%) | 5 (16.7%) | 11 (36.7%) |
| Relapse with impaired capacity to | 1 (3.3%) | 5 (16.7%) |
| control the amount of alcohol taken | | |
| (%) | | |
| Relapse with loss of quantitative and | 0 | 4 (13.3%) |
| situational control of alcohol taking | | |
| (%) | | |

There were apparent differences between the study groups. Single-instance consumption was reported for 16.7% of patients in the group of AP drug. In the placebo group, occasional (episodic) alcohol consumption was observed in 16.7% of patients; at the same time 13.3% of patients developed relapses with impaired capacity to control the amount of alcohol consumed, 36.7% had relapses with loss of quantitative control, and 13.3% of patients were incapable to control both the amount and situations of alcohol taking. Five subjects in the placebo group dropped out of the study due to relapses. In the group of AP drug, all patients completed the study. In cases of relapse, the latter was stopped with an increased dose of test drug—up to 12 tablets daily.

Efficacy Conclusions.

1. AP drug was shown to have high clinical efficacy when used as a monotherapy in patients with stage 2 alcohol dependence syndrome. The 24-week treatment with AP drug resulted in statistically significant health improvements in the above patient category, as assessed clinically.
2. AP drug reduces the occurrence of relapses in alcoholic patients developing remission; with an increased dose (up to 8-12 tablets daily), it helps in stopping the renewed appetence for alcohol in relapses. The clinical effect of AP drug is higher than that of the comparator drug (placebo).
No adverse events were reported for by far the majority of patients receiving the study treatment. All patients in the group of AP drug completed the treatment in due time, as scheduled in the study protocol (after 24 weeks, respectively). Five patients in the placebo group dropped out of the study due to relapsing into alcohol dependence. The treatment was not associated with any complaints or development of somatic disturbances. All patients reported good tolerance of the drugs received.

### Example 2

An open, non-comparative study was conducted to examine the efficacy and safety of the claimed drug, formulated as a water-alcohol solution (liquid dosage form) of the activated (potentized) energy-treated carrier, obtained by extreme serial dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a difference of potentials being applied to electrodes placed in the initial liquid carrier and which is proportional in amplitude to an amplified combined, summed signal, formed by summing the resultant signals—the differences of biopotentials of individual referential derivations recorded in five healthy donors using the internationally recognized 10-20 system, which involves 19 active recording electrodes placed in regular positions all over the scalp and one reference electrode located on the ear-lobe)—further referred to as AP drug), where it was used as part of a combination treatment for negative (deficit-type) syndrome in patients with paranoid (hallucinatory-delusional) schizophrenia.

The study involved 20 patients (women between 48-70 years old; mean age range, 51.1±1.3 years) presenting to the inpatient psychiatric unit with exacerbated psychotic symptoms requiring anti-psychotic treatment.

The patients underwent an objective assessment procedure: mental health screening using the Brief Psychiatric Rating Scale (BPRS) Special consideration was given to the BPRS items that reflect disturbances of emotional tone and volition as well as conceptual disturbances, which are typical of schizophrenia-related deficit syndrome:

3. Emotional withdrawal. Lack of spontaneous interaction, isolation deficiency in relating to others during the procedure
4. Conceptual disorganization. The extent to which the thought process is confused, disconnected or disrupted.
7. Mannerism and posturing. Peculiar, bizarre, unnatural motor behavior.
13. Motor retardation. Slowed, weakened movements, reduced body tone.
16. Blunted affect Reduced emotional tone, reduction in formal intensity of feelings, flatness.
In addition, occurrence of adverse events during the treatment was recorded, including the nature and causal relationship thereof with the test drug.

The following efficacy endpoints were analyzed:

1. Severity reduction of negativism symptoms within 6 hours of observation;
2. Score changes in items 3, 4, 7, 13, 17 of the BPRS inventory as assessed after 6 hours of observation.
3. Score changes in items 3, 4, 7, 13, 17 of the BPRS inventory as assessed at 14 and 28 days of observation.
Dosing regimen employed: 20 oral drops 6 times at 1-hour intervals on day 1, followed by 20 oral drops 4 times daily for the rest of the days (a total of 28 days). Observation period: 28 days

Concomitant Medications:

1. The patients were allowed to use medications for co-existing somatic conditions throughout the study period.
2. AP drug was allowed to be used concomitantly with previously prescribed antipsychotic drugs (neuroleptics: haloperidol, aminazine, azaleptin, triftazin, tisercin, moditen depo, serdolect, torendo; and neurolepsy corrector—trihexyphenidyl). The exclusion criterion was sufficient duration of previous treatment, which had not been modified for at least 1 month, with the achieved mental state remaining stable for over 2 weeks prior to AP addition. However no new prescriptions of phychoactive drugs were permitted!

Study Design:

The deficit symptoms (emotional deficiency, motor retardation, blunted affect) were assessed by the clinician at hour's intervals over 6 hours after the administration of test drug, which was monitored for effects on patients' waking state (potential hypnotic effect); at the same time the BPRS was administered and observations diary completed. Further mental assessments using the BPRS were performed at 14 and 28 days.

All patients enrolled had previously presented to the inpatient psychiatric unit with a diagnosis of ‘Schizophrenia, paranoid type, paranoid syndrome. Continuous or episodic course with progressive deficits. Severe emotive and volitional defects’. In order to eliminate productive psychotic symptoms and prevent hostile affect, the inpatients admitted were administered conventional and atypical neuroleptics: haloperidol—4 patients (20%), aminazine—1 patient (5%), azaleptin—8 patients (40%), triftazin—2 patients (10%), tisercin—3 patients (15%), moditen depo—2 patients (10%), etaperazine—1 patient (5%); serdolect—1 patient (5%), torendo—3 patients (15%); zalasta—1 patient (5%); and to prevent neuroleptic complications—trihexyphenidyl (14-patients (70%)).

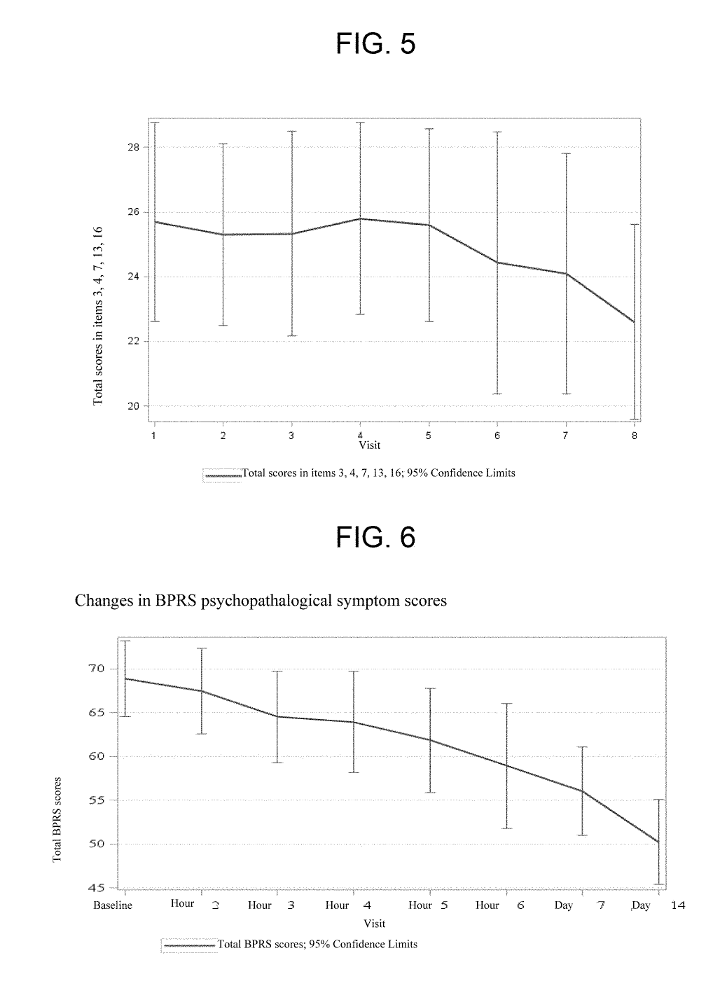

The analysis of treatment efficacy based on the above-mentioned BPRS items 3, 4, 7, 13 and 16 (FIG. 5) showed that the negative symptoms (animosity, negativism, motor retardation) were significantly (p<0.0001) reduced following only 6 hours after the initial doses of test drug (20 drops every hour). Most patients (18 subjects) fell asleep after the 1st-2nd dose of AP drug and slept for 3-4 hours (Table 4). After awaking, they were subjectively calmer, emotionally “milder”, demonstrating less tension and belligerence (a clear positive trend was observed in the total score reduction for items defining negativism symptoms—from 23.4±5.8 to 22.4±6.1 (Visit 1-6; p=0.0632) (Table 5).

TABLE 4
Hypnotic effect of AP drug as observed within the first 6 hours

| Parameter | Hours |
|---|---|
| Sleep latency | 4.33 ± 1.72 |
| (n = 18) | (2.0-6.0) |
| Sleep duration | 1.58 ± 0.79 |
| (n = 18) | (1.0-3.0) |

TABLE 5
BPRS psychopathalogical symptom scores in the course of treatment

| Parameter | Total score | Statistics |
|---|---|---|
| Total score for | | Factor ‘Visit (1-8)’: |
| items 3, 4, 7, 13, 16 | | F_{7/119} = 7.2; |
| | | p < 0.0001 |
| Baseline | 23.4 ± 5.8 | Factor ‘Visit (1-6)’: |
| 6 hours | 22.4 ± 6.1 | F_{5/81} = 2.2; |
| Day 14 | 21.7 ± 6.3 | p = 0.0632 |
| Day 28 | 20.4 ± 5.8 | |

Significant and quite sustainable changes were observed in the patients' mental health, as reported by the unit's staff, by day 14 (Visit 7) of the treatment (total score reduction for negativism symptoms related items—from 23.4±5.8 to 21.7±6.3; p<0.0001). First, the clinicians and stuff member reported emotional responses of the patients to the treatment conducted, such as improved contact: The patients kept longer eye contact and behaved more amicably while talking with the doctor. Relatives also noted improved ‘amicability and warmth’: no aggression or negativism, more liveliness and emotional “warmth” were demonstrated by the patients when seeing the former. One of the patients had been moody, been walking around with her head down, been sluggish in responding to questions and untidy in appearance, and had not socialized with anyone during her previous stay at the unit (before being treated with AP drug). Within the first few hours of treatment, the psychic tension was lowered as seen during conversations with the doctor. The patient began to show more animated facial expressions and exhibit more vivid emotions; her movements were less rigid and accompanied by more gesturing. She also began to show concern about own appearance, looked satisfied when seeing herself in the mirror, and began to engage with the unit's chores. She had a good night's sleep. Less pronounced positive changes were observed in cognitive performance, and conceptual disturbances (long-existing paralogisms, disconnected, disorganized, disrupted thought process. However, these disturbances were less severe due to improved patients' capacity to interact with others and reduced affective responsiveness.

There were also statistically significant changes in negative symptoms, as evidenced by differences in the mental health of patients at baseline compared to that after 28 days (Visit 8) of treatment with AP drug (the total negative symptoms score reduced from 23.4±5.8 to 20.4±5.8; p<0.0001).

The treatment involving AP drug resulted, as judged by the doctor, in reduced severity of emotive and volitional defects in 15 patients (75%) and did not lead to any clinically relevant deficit syndrome changes in 5 patients (25%). It should be noted that no mental health worsening or increase in negative symptom manifestations was reported for any of the patients.

### Conclusions

All patients enrolled had more or less severe negative (deficit) symptoms, which were observed to evaluate disease progression and were principally assessed by recording changes in the inpatients' behavior, interaction with other people and adaptation at the psychiatric unit. The test drug was administered in combination with antipsychotic medications, the latter proving to have little effect on the negative symptoms (as indicated by medical history data from previous hospitalizations). In the majority of patients, AP drug, used in the assessment of clinical symptom changes over time (in comparison with the preceding treatment), was associated, in addition to reduced severity of affective disturbances of psychosis, with notable emotional tone effects, which were already observable within the first few hours of treatment. Each dose of the test drug was seen to ease affective responsiveness and belligerence in patients. Although no rectification of the conceptual defects was observed as a result of study treatment, these disturbances were less pronounced as expressed by the patients' behavior and adaptation at the psychiatric unit, which was achieved through improved emotional tone and better patients' capacity to interact with others. The increase in emotion intensity was not associated with exacerbation of paranoid syndrome (hallucinatory or delusional symptoms) in any of the patients and did not require adjustment of medications administered. On the whole, the test drug's effects on the inpatients' behavior proved to be beneficial, as objectively evidenced by the general environment in the psychiatric unit.

Thus, AP drug, added to the neuroleptic treatment in patients with emotive and volitional disturbances due to an endogenous disease (schizophrenia), was shown to reduce affective manifestations of the emotional defect immediately after the first doses thereof. A notable improvement of the patients' mental health, expressed as better capacity for spontaneous interaction and normalized behavior, was recorded after two weeks of treatment and was more sustainable by the end of week 7. The test drug has a beneficial effect on patients' behavior in the settings of a psychiatric unit, and could be effective in alleviating social maladaptation and lengthening remission at later time points.

### Example 3

An open, non-comparative study was conducted to examine the efficacy and safety of the claimed drug, formulated as a water-alcohol solution (liquid dosage form) of the activated (potentized) energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial liquid carrier has been accomplished by exposing it to an electric current caused by the difference of potentials being applied to electrodes placed in the initial liquid carrier and which is proportional in amplitude to an amplified combined, summed signal, formed by summing up the resultant signals—differences of biopotentials of individual referential derivations recorded in five healthy donors using the internationally recognized 10-20 system, which involves 19 active recording electrodes placed in regular positions all over the scalp and one reference electrode located on the ear-lobe)—further referred to as AP drug, where it was used for treating acute psychotic disorder (hallucinatory-delusional syndrome) of endogenous origin.

The study involved 20 patients (women between 18-65 years old; mean age range, 49.1±13.3 years) presenting to the inpatient psychiatric unit with exacerbated psychotic symptoms requiring anti-psychotic treatment.

The patients underwent objective assessment procedure: mental health screening using the Brief Psychiatric Rating Scale (BPRS)

Occurrence of adverse events during the treatment, including the nature and causal relationship thereof with the test drug.

Efficacy Endpoints:

1. Severity reduction or reversal of the psychotic disorder within 6 hours of observation; BPRS score changes after a 6 hours of observation. BPRS score changes at 7 and 14 days of observation.
2. Reduction of productive symptoms (hallucinations and delusional statements). Reduced severity of affective manifestations in psychotic experiences at 7 and 14 days of observation. Secondary efficacy endpoints: development of hypnotic effect within 6 hours after taking the test drug; sleep latency time (the time from the first dose to getting to sleep); duration of sleep; severity of psychotic symptoms after awaking;

Dosing regimen employed: 20 oral drops 6 times at 1-hour intervals on day 1, followed by 20 oral drops 4 times daily for the rest of the days (a total of 14). Observation period: 14 days.

Concomitant Medications:

1. The patients were allowed to use medications for co-existing somatic conditions throughout the study period.
2. AP drug was allowed to be used concomitantly with previously prescribed antipsychotic drugs (neuroleptics: haloperidol, aminazine, azaleptin, triftazin, tisercin, moditen depo, serdolect, torendo; and neurolepsy corrector—trihexyphenidyl). The exclusion criterion was sufficient duration of previous treatment, which had not been modified for at least 1 month, with the achieved mental state remaining stable for over 2 weeks prior to AP addition. However no new prescriptions of phychoactive drugs were permitted!

Study design: psychopathological symptoms—affective (psychomotor agitation) and productive (hallucinatory-delusional)—were assessed by the clinician at hour's intervals over 6 hours after the administration of test drug, which was also monitored for effects on patients' waking state (potential hypnotic effect); at the same time the BPRS was administered and observations diary completed. Further mental assessments on the BPRS were performed at 7 and 28 days.

During admission to the psychiatric unit, the patients were screened for somatic pathology. The screening results indicated the presence of cardiovascular disorders (ischemic heart disease, aortic and coronary atherosclerosis, stage 2 hypertension) in 4 patients (20%), respiratory disease (chronic bronchitis in remission) in 1 patient (5%), and gastrointestinal disease (chronic pancreatitis in remission) in 1 patient (5%).

Two study subjects were admitted to the inpatient psychiatric unit for the first time. The other 18 patients had previously presented to a psychiatric facility.

The prevailing diagnosis was ‘schizophrenia, paranoid type, paranoid syndrome. Continuous course with progressive deficits.

The second most common diagnosis was ‘organic brain disease of complex origin (toxic, vascular), hallucinatory-paranoid syndrome’

As recorded at baseline, most patients had been taking antipsychotic medications (conventional and new-generation neuroleptics), such as: haloperidol—4 patients (20%), aminazine—1 patient (5%), azaleptin—8 patients (40%), triftazin—2 patients (10%), tisercin—3 patients (15%), moditen depo—2 patients (10%), etaperazine—1 patient (5%); serdolect—1 patient (5%), torendo—3 patients (15%); zalasta—1 patient (5%); and neurolepsy corrector—trihexyphenidyl (14-patients (70%)).

The treatment efficacy analysis using the BPRS inventory identified a significant (p<0.0001) severity reduction of the psychotic symptoms as early as within 6 hours of initial doses of the test drug (20 drops every hour) (Table 6).

TABLE 6
Psychopathological symptom scores as measured by the BPRS at
different time points of treatment

| Parameter | AP | Statistics |
|---|---|---|
| Overall score, all patients | | |
| Baseline [20] | 68.9 ± 9.3 | Factor ‘Visit (1-8)’: |
| Hour 2 [20] | 67.5 ± 10.5 | F_{7/119} = 11.1; |
| Hour 3 [18] | 64.5 ± 10.6 | p < 0.0001 |
| Hour 4 [17] | 63.9 ± 11.2 | Factor ‘Visit (1-6)’: |
| Hour 5 [18] | 61.8 ± 11.9 | F_{5/81} = 7.0; |
| Hour 6 [13] | 58.9 ± 11.8 | p < 0.0001 |
| Day 7 [20] | 56.1 ± 10.7 | Factor ‘Sleep (1-6)’: |
| Day 14 [20] | 50.3 ± 10.4 | F_{1/18} = 0.09; |
| | | p = 0.77 |

Almost all patients reported relaxation and sensation of calm after first doses of test drug. Eighteen patients fell asleep within 2-4 of the first dose of AP drug, and two patients were dozing off (Table 7; FIG. 6)

TABLE 7
Hypnotic effect within 6 hours of observation

| Parameter | Hours |
|---|---|
| Sleep latency | 4.33 ± 1.72 |
| (n = 18) | (2.0-6.0) |
| Sleep duration | 1.58 ± 0.79 |
| (n = 18) | (1.0-3.0) |

After awaking, all patients demonstrated less severe affective manifestations of hallucinatory-delusional experiences and reduced or eliminated anxiety or depression symptoms. The differences in patients' health status between baseline and 6 hours after the initiation of AP were statistically significant, as shown by the overall evaluation results of all patients and individual results of patient who were able to fall asleep and those remaining awake. The differences compared to baseline as evaluated at 14 days after test drug initiation were significant (p<0.0001) in all patient subsets analyzed.

The treatment involving AP drug resulted in mental health improvement in 18 patients (90%), and did not lead to any clinically relevant changes in only 2 patients (10%). However, it should be noted that no mental health worsening was reported for any of the patients, which, compared with the medical history data from previous hospitalizations, had a beneficial effect on disease progress.

Although the test drug was administered concomitantly with neuroleptic medications, with regard to comparative assessment of clinical symptoms over time (in comparison with the preceding treatment), of the most importance is its beneficial effects on patients' affective status, which was significantly improved within the first few hours of treatment initiation. Each intake of the test drug was seen to ease affective responsiveness and belligerence in patients.

Although the productive symptoms were not completely resolved by the study treatment, the severity of affect expression was considerably lower, which had an ameliorating effect on the hallucinatory and delusional experiences. Since the duration of previous psychotic episodes during first admissions to the psychiatric unit was 4 months, compared to less than 1 month in the present study, the resolution of pathological symptoms can not be attributed to spontaneous cessation of the psychotic attacks. On the whole, the test drug's effects on the inpatients' behavior proved to be beneficial: the patients demonstrated better capacity for social interaction and were calmer and more amicable, which showed on the general environment in the psychiatric unit.

Of special note are the favorable effects of AP drug on adverse events (AE)—neuroleptic symptoms caused by the neuroleptic drugs administered: these AEs were considerably reduced within 3-7 days of concomitant AP administration in most patients, and three patients, who had been using neuroleptics as the primary treatment, did not develop any neurolepsy symptoms. In general, the neuroleptic therapy was well tolerated by the patients, as compared to the previous inpatient treatments. None of the patients developed AEs that could be related to AP proper.

Thus, as a result of AP drug, added to the neuroleptic treatment in patients initially or repeatedly admitted to the psychiatric unit, the latter demonstrated normalized sleep and notable improvement of mental health within the first day of study treatment, and a significant reduction of the affective manifestations of psychotic symptoms at two weeks after treatment initiation. The test drug has a beneficial effect on a psychiatric unit's environment, contributes to more adequate self-assessment of disease, reduces the level of social maladaptation, increases emotional liveliness, and prevents the development of neuroleptic complications.

### Example 4

A randomized, double-blind, placebo-controlled study was conducted to examine the efficacy and safety of the claimed drug, formulated as a solid dosage form containing lactose granules impregnated with water-alcohol solution of the liquid activated (potentized) energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial liquid carrier has been accomplished by exposing it to an electric current caused by a difference of potentials being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug, where it was used for treating depression in patients with post-traumatic stress disorders.

Post-traumatic stress disorders (PTSD) occur as a response to mental or physical stress, which is likely to cause pervasive distress in almost anyone. Such traumas may arise in fights, natural disasters, attacks by criminals, and emergencies such as household fires. Unlike an acute stress reaction, a PTSD does not occur at the time of the stressful situation, but arises as a delayed response to the event—after the stressful situation has resolved. The onset follows the trauma with a latency period that may range from a few weeks to months (usually not more that 6 months).

Conventional benzodiazepine anxiolytics had demonstrated insufficient efficacy in this patient group. First, the numerous side effects associated with these drugs turn patients away from undergoing treatments prescribed; secondly, the risk of tolerance development prevents tranquilizers from being used for long-term courses, and the high likelihood of withdrawal syndromes leads to a loss of trust between the patient and the doctor. Antidepressants of different groups are also likely to cause adverse events affecting the central nervous system and somatic health.

AP drug was administered to manage the depressive symptoms—in parallel to Heptral (adametionine), used as a comparator in efficacy assessment. AP drug was administered for 4 weeks at a dose of 2 tablets 4 times daily. Heptral was used at 400 mg 3 times (1200 mg) daily.

An open comparative clinical trial was performed which involved 30 patients (16 men and 14 women) assigned to two parallel groups: 15 patients in the group of AP drug and 15 in the heptral group. The mean age of participants assigned to AP drug was 38.0±4.39 years;

The period of study participation was 8 weeks (the total treatment duration of 4 weeks and 4 weeks of follow-up).

Neither of the patient groups was allowed to take psychoactive medications during the study. The drugs not prohibited for use during the treatment were medications for the underlying traumatic illness and co-existing somatic disorders. The study enrolled inpatients aged from 18 to 65 years old, who had been receiving medical attention for bodily injuries resulting from car accidents, which had taken place under circumstances threatening the life or welfare of the patient or/and their friends or family members suffering from anxiety mental disorders, such as (defined by the ICD-10) reaction to severe stress, and adjustment disorders (F43); adjustment disorders (F43.2); adjustment disorder with mixed anxiety and depressed mood (F43.23).

Severe clinical anxiety and depression corresponded to a total HAM-D (Hamilton Depression Rating Scale) score of ≧20, and HADS (Hospital Anxiety and Depression Scale) anxiety and depression subscores of ≧11.

The efficacy endpoints were:

1. Reduced severity of depressive symptoms and signs as measured by the HADS.
2. Reduced severity of depressive symptoms and signs as measured by the HAM-D
3. Efficacy Index assessment on the Clinical Global Impression (CGI) scale.

The safety endpoint was the occurrence and nature of adverse events (AE) as observed during the treatment.

The patients' mental health was examined at baseline—at admission to the clinic, following 1, 2 and 4 weeks of treatment, and 4 weeks after treatment discontinuation (follow-up).

The patients were assessed against the inclusion/exclusion criteria, and allocated to treatment groups as follows: AP drug: 15 subjects; Heptral: 15 subjects.

### Efficacy Evaluation

The effect of study treatment was evaluated according to changes in the total HAM-D score and scoring outcomes on the HADS anxiety and depression subscales. The evaluation results are summarized in Table 8 and 9. In addition, the Clinical Global Impression scale was administered to primarily estimate the Efficacy Index for the treatment conducted.

TABLE 8
Total HAM-D scores in patients groups

| | Visit 1 | Visit 2 | Visit 3 | Visit 4 | Visit 5 |
|---|---|---|---|---|---|
| Group | (baseline) | (week 1) | (week 2) | (week 4) | (follow-up) |
| AP | 25.7 ± | 16.1 ± | 10.1 ± | 7.2 ± | 1.6 ± |
| (n = 15) | 1.26 | 0.90** | 0.70*** | 0.70*** | 0.43*** |
| Heptral | 26.2 ± | 23.1 ± | 15.6 ± | 14.1 ± | 12.1 ± |
| (n = 15) | 1.30 | 0.85* | 0.83*** | 0.40*** | ,0.23*** |

TABLE 9
Total HADS depression scores in patients groups

| | Visit 1 | Visit 2 | Visit 3 | Visit 4 | Visit 5 |
|---|---|---|---|---|---|
| Group | (baseline) | (week 1) | (week 2) | (week 4) | (follow-up) |
| AP | 18.9 ± | 15.0 ± | 10.7 ± | 5.9 ± | 3.2 ± |
| (n = 15) | 0.64 | 0.95** | 0.96*** | 0.65*** | 0.50*** |
| Heptral | 17.5 ± | 13.2 ± | 9.6 ± | 6.5 ± | 3.5 ± |
| (n = 15) | 0.60 | 0.71** | 0.74*** | 0.42*** | 0.53*** |

Note:
*p as compared to baseline <0.05;
**p as compared to baseline <0.01;
***p as compared to baseline <0.001;
°p as compared to heptral <0.05;
°°p as compared to heptral <0.01;
°°°p as compared to heptral <0.001.

The evaluation results of the HAM-D scores indicate that by week 4 no depression episodes were developed by patients in either group, with no significant differences between the groups. Similar results were provided by the HADS subscale for depression (normal depression scores were achieved by week 4 in both groups, with no significant between-group differences).

As in the case of anxiety evaluation, the follow-up data analysis (Visit 5) shows that the above assessment parameters continued to significantly decrease (p<0.001) in both groups through the 4 weeks after treatment discontinuation.

All CGI scores analyzed were significantly improved in both groups (p<0.01, p<0.001) already after two weeks of treatment. The positive changes in CGI assessments were observed up to the end of week 4 and onwards through the follow-up period (Table 10).

TABLE 10
Total score outcomes on the CGI subscales

| | | Visit 1 | Visit 2 | Visit 3 | Visit 4 | Visit 5 |
|---|---|---|---|---|---|---|
| | Group | (baseline) | (week 1) | (week 2) | (week 4) | (follow-up) |
| Severity | AP (n = 15) | 3.2 ± 0.22 | 2.6 ± 0.16** | 2.2 ± 0.14** | 1.3 ± 0.16** | 1.0 ± 0.00*** |
| scale | Heptral (n = 15) | 3.8 ± 0.17 | 2.9 ± 0.12** | 1.9 ± 0.12** | 1.1 ± 0.07*** | 1.0 ± 0.00*** |
| Improvement | AP (n = 15) | | 3.1 ± 0.13 | 2.2 ± 0.20** | 1.5 ± 0.13*** | 1.1 ± 0.09*** |
| scale | Heptral (n = 15) | | 2.9 ± 0.07 | 2.1 ± 0.15** | 1.2 ± 0.11*** | 1.1 ± 0.07** |
| Efficacy | AP (n = 15) | | 1.8 ± 0.19 | 2.7 ± 0.22** | 3.6 ± 0.21*** | 3.6 ± 0.21*** |
| Index | Heptral (n = 15) | | 1.1 ± 0.08 | 1.8 ± 0.19*** | 2.3 ± 0.18*** | 2.3 ± 0.18*** |

Note:
*p as compared to baseline <0.05;
**p as compared to baseline <0.01;
***p as compared to baseline <0.001;

Noteworthy, the CGI Efficacy Index scores were significantly higher in the group treated with AP drug than in the heptral group, which is chiefly associated with better AP drug's safety coupled with almost identical efficacy.

Safety Evaluation:

Antidepressant effect of AP drug was demonstrated in patients with stress-related depression and anxiety disorders, and those at risk for PTSD, who received the AP drug at 2 tablets 4 times daily for 4 weeks. As a result of treatment with AP drug, the analyzed parameters were significantly improved, as evidenced by: total HAM-D score reduction from 25.7±1.26 to 1.6±0.43 (significance level for differences between mean total scores in the groups: p>0.05), total HADS depression score reduction from 18.9±0.64 to 3.2±0.50 (significance level for differences between mean total scores in the groups: p>0.05), indicating that the antidepressant effects of AP drug were similar to those of heptral and were preserved 4 weeks after treatment discontinuation; and by positive changes in Efficacy Index assessments, which were significantly (p<0.01) more pronounced in the group of AP drug as compared to the heptral group.

Safety Evaluation:

The safety analysis included data of all study participants (n=30). Treatment with AP drug was not associated with any mental health or organ-specific adverse events,

whereas in the heptral group, adverse events expressed as dizziness, headache, and anxiety worsening were recorded in 5, 3, and 2 patients, respectively.

Thus, the results obtained show that:

1. AP drug has a significant antidepressant effect, resulting in PTSD risk reduction. AP drug administered at 2 tablets 4 times daily was as effective in reducing anxiety and depression symptoms as heptral. CGI Efficacy Index scores were significantly higher in the group of AP drug compared to the heptral group.
2. AP drug is safe when administered at 8 tablets daily for 4 weeks: no adverse events were developed by any of the patients.

AP drug provides effective and safe treatment for patients with stress-related (reactive) depression and anxiety disorders.

### Example 5

A comparative study was conducted to examine the efficacy and safety of the claimed drug, formulated as a solid dosage form containing lactose granules impregnated with water-alcohol solution of the liquid activated (potentized) energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a difference of potentials being applied to electrodes placed in the initial neutral carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug, where it was used for treating anxiety in patients with neurotic disorders (ND), and attention deficit hyperactivity disorder in children.

The study included 57 children aged from 6 to 9 years (40 boys and 17 girls), who were nursery/preschool attendants and primary school students.

The inclusion criteria were:
1. Children with clinical signs and symptoms of a hyperkinetic disorder (HD) meeting the ICD-10 criteria (F 90);
2. Age range: 6 to 9 years;
3. At least a medium IQ score (90 points) on the Wechsler Intelligence Scale.

The study excluded children with behavior disturbances related to other mental disorders. Children with HD signs and symptoms who were allocated to the group of test treatment (n=30) received AP drug as a monotherapy, at 2 tablets 3 times daily for 2 months. The participants in the control group, comparable to the above group in clinical signs and symptoms, age and sex distribution, were assigned to receive piracetam at standard age-specific doses.

HD severity was assessed by attention, hyperactivity and impulsivity scores on the SNAP-IV Rating Scale (Swanson J., 1992). The psychophysiological TOVA test (Test of Variables of Attention) was used to measure attention deficits (omission errors) and impulsivity (commission errors) as well as the speed of information processing (response time) as compared to the normal ranges (Greenberg L. M., Waldman I.D., 1993). The children's fine motor skills were measured by tests from A. R. Luria's test battery (1973), which had been tried out in the same age groups using the scoring criteria designed by J. M. Glozman et. al. (2006).

Study Results:

In the initial stage, the children in the test and control groups were administered the SNAP-IV (Swanson J., 1992) for a baseline assessment of HD symptoms according to the three different scores (attention, hyperactivity and impulsivity). This assessment was repeated following two months of treatment with AP drug (test group) and piracetam (control group). The scores obtained were compared with the normative rating results and statistically analyzed (Table 11).

TABLE 11
SNAP-IV scores in patient groups following treatments with AP drug
and piracetam, respectively.

| | Children | | Control | Normative |
|---|---|---|---|---|
| | with HD | Test | group | scores |
| | baseline | group (AP) | (piracetam) | (Swanson J., |
| Parameter | (n = 27) | (n = 30) | (n = 57) | 1992) |
| Attention | 3.15 ± 0.32 | 1.72 ± 0.12** | 2.48 ± 0.12 | 0.40 ± 1.82 |
| problems | | | | |
| Hyperactivity | 2.82 ± 0.31 | 1.32 ± 0.21** | 2.41 ± 0.10 | 0.27 ± 1.59 |
| Impulsivity | 2.84 ± 0.278 | 1.62 ± 0.34* | 2.28 ± 0.22 | 0.26 ± 1.52 |

*p < 0.05,
**p < 0.01—significant differences in comparisons between baselin and treatment completion

As seen from Table 11, the test treatment with AP drug resulted in a significantly improved attention, reduced hyperactivity and impulsivity (less markedly) in most patients—21 (70%, n=30). Of note is the high significance of differences between the scores obtained in the test group before and after AP drug treatment, indicating its beneficial effect. In the control group, piracetam treatment was associated with improved scores but no significant differences were recorded for the variables compared.

The computer-based psychophysiological TOVA assessment involved three sets of tests measuring inattention (omission errors), impulsivity (commission errors), and speed of information processing (response time). The baseline measurements of HD children were compared with those obtained in the test group (AP drug) and in the control group (piracetam) after two months of treatment. The results are summarized in Table 12.

TABLE 12
Comparison of TOVA scores recorded before and after study treatment
in the AP and piracetam groups

| | | Children | | Control |
|---|---|---|---|---|
| | | with HD | Test | group |
| Psychophysical | | baseline | group (AP) | (piracetam) |
| variables | | (n = 57) | (n = 30) | (n = 27) |
| Omission | First | 18.2 ± 2.7 | 7.9 ± 1.1** | 13.6 ± 1.4* |
| errors, | section | | | |
| % | Second | 28.15 ± 3.2 | 9.7 ± 1.3** | 17.2 ± 2.8* |
| | section | | | |
| Commission | First | 17.3 ± 2.1 | 7.2 ± 1.7* | 12.5 ± 3.2 |
| errors, | section | | | |
| % | Second | 31.2 ± 3.0 | 15.8 ± 1.8* | 21.1 ± 2.7 |
| | section | | | |
| Response | First | 763 ± 101 | 717 ± 88 | 736 ± 83 |
| time, | section | | | |
| ms | Second | 742 ± 121 | 621 ± 75 | 656 ± 79 |
| | section | | | |

*p < 0.05,
**p < 0.01—significant differences in comparisons between baselin and treatment completion

As shown by Table 12, the psychophysiological measurements of attention (from testing the omission of targets) were significantly improved in both study groups. However, significant reduction in impulsivity (commission errors) was only achieved in the test group (AP drug). In total, the psychophysiological variables were improved as a result of AP treatment in 16 children (53.3%, n=58) in the test group. Neither AP nor piracetam demonstrated statistically significant effects on the promptness of decision making (response time). Thus, the psychophysiological tests proved supportive of the clinical data that provide evidence of AP drug's beneficial effect on attention and mental performance. It was also shown to result in impulsivity reduction. Neuropsychological assessment of fine motor skills as performed after treatment completion similarly indicated statistically significant improvements for both study groups. The results are summarized in Table 13.

TABLE 13
Measurements of fine motor skills in study groups as obtained after
treatment completion.

| | Children | | Control |
|---|---|---|---|
| | with HD | Test | group |
| | baseline | group (AP) | (piracetam) |
| Parameter | (n = 57) | (n = 30) | (n = 27) |
| Total score | 2.24 ± 0.21 | 1.56 ± 0.21 ** | 1.62 ± 0.16* |
| for fine motor | | | |
| skills | | | |

*p < 0.05,
**p < 0.01—significant differences in comparisons between baselin and treatment completion

It should be specifically noted that the treatment with AP drug was not associated with any side effects or complications; whereas piracetam resulted in increased disinhibition/excitability in 3 children (11.1%) and sleep problems (difficulties getting to sleep, waking up frequently) in 7 (26%) young participants.

### Conclusion

The comparative efficacy study of 2-months' treatment with AP drug in children with hyperkinetic disorders demonstrated improved children's behavior and significantly better attention and response time outcomes. In addition, there was a reduction in hyperactivity and (to a lesser extent) impulsivity. The efficacy of AP drug in attenuating ADHD symptoms was significantly higher compared to piracetam. Although improvement of fine motor skills was observed in both study groups, more marked beneficial changes in this parameter were demonstrated by children in the test group. The 2-month administration of AP drug was not associated with side effects such as disinhibition, hyperkinesia, or sleep disturbances, which were reported in the group of piracetam treatment.

### Example 6

An open, non-comparative study was conducted to examine the efficacy and safety of the claimed drug, formulated as a water-alcohol solution (liquid dosage form) of the activated (potentized) energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial liquid carrier has been accomplished by exposing it to an electric current caused by a difference of potentials being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug, where it was used for treating cerebral palsy.

The study included 7 participants with different types of cerebral palsy: spastic tetraplegic, hyperkinetic, atonic-astatic, and hemiparetic cerebral palsies; several patients were diagnosed with delayed speech development.

The patients were prescribed to take AP drug at 10 drops 6 times daily for 1 week, followed by 10 drops 3 times daily for weeks 2 through 8. The total study period was 2 months (8 weeks).

Impairments of motor functions were assessed by changes in scores on the GMFM-66 (Gross Motor Function Measure) inventory.

The mean age of the patients enrolled was 3.8±0.46, of whom 6 subjects were males and 1 was a female. The treatment outcomes are shown in Table 14.

TABLE 14
GMFM-66 scores of study participants as recorded before and after the course of
treatment.

| | | GMFM 1 | GMFM 2 | |
|---|---|---|---|---|
| | Pre- | month | months | |
| Patient | treatment | post | post | |
| ID | GMFM | treatment | treatment | Subjectively |
| 001 | 25 | 29 | 32 | Improved muscle tone and sitting posture; the |
| | | | | child is calmer and demonstrates better ability to |
| | | | | adjust to new environment |
| 002 | 117 | 141 | 152 | Improved muscle tone, increased range of |
| | | | | movements of some joints, reduced ataxia, |
| | | | | improved fine motor skills, better verbal |
| | | | | comprehension, enlarged vocabulary, better |
| | | | | attention switching |
| 003 | 133 | 137 | 138 | Increased physical strength, reduced pseudobulbar |
| | | | | symptoms, improved adjustment to environmental |
| | | | | changes, lower irritability |
| 004 | 130 | 138 | 146 | Improved muscle tone, increased range of |
| | | | | movements of some joints, better ability to |
| | | | | maintain focus and adjust to new environment. |
| | | | | Enlarged vocabulary |
| 005 | 50 | 60 | 60 | Improved muscle tone, less joint stiffness, |
| | | | | reduced ataxia and propulsion when walking |
| | | | | Steadier walking, able to walk longer distances. |
| 006 | 125 | 131 | 132 | Good environmental adjustment, improved |
| | | | | muscle tone, slightly reduced dysphonia |
| 007 | 146 | 152 | 165 | Improved muscle tone, increased range of |
| | | | | movements of some joints, better verbal |
| | | | | comprehension, enlarged vocabulary. |

The treatment with the test drug was combined with a large range of rehabilitation interventions, such as physiotherapeutic procedures, speech therapy, therapeutic exercise, massage.

Overall, a beneficial effect was observed mostly on the muscle tone, coupled with an increased range of joint movements; two patients exhibited reduced pseudobulbar symptoms. In addition, most patients demonstrated better adjustment outcomes and improved verbal production and comprehension.

There was a delayed effect of the drug treatment: the patients who continued the test drug after being discharged from the center were able to maintain the achieved effects for longer periods of time as compared to earlier follow-up results for these patients.

### Example 7

In an open, non-comparative study, the claimed drug, formulated as a solid dosage form containing lactose granules impregnated with water-alcohol solution of the liquid activated (potentized), energy-treated carrier in an ultra-low dose, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a difference of potentials being applied to electrodes placed in the initial neutral carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug—was examined for efficacy and safety in treating sequelae of ischemic cerebrovascular accident (CA).

The study participants were 10 subjects who had a history of ischemic stroke experienced at different periods of life prior to this study and associated with residual disability (mRS (modified Rankin Scale) scores of 3-4).

The patients were prescribed to take AP drug as a monotherapy, at 2 tablets*3 times daily for 28 days.

The participants were assessed for severity of functional disability by comparing baseline mRs scores with those recorded following 4 weeks of test treatment.

The mean age of patients was 66.1±4.01 years, the sex distribution being 6 men and 4 women. The disease age ranged from 0.9 to 9 years (the average of 2.48 years). The results of study treatment are summarized in Table 15.

TABLE 15
mRS scores of study patients as recorded prior to and after the
monotherapy with AP drug

| | Pre- | Post- | |
|---|---|---|---|
| Patient | treatment | treatment | |
| ID | mRS | mRs | Subjectively |
| 001 | 3 | 2 | Improved ability to attend to own needs |
| 002 | 4 | 4 | Improved general health, reduced |
| | | | headache |
| 003 | 3 | 3 | No changes |
| 004 | 3 | 3 | Improvements in general health, |
| | | | exercise tolerance and mood* |
| 005 | 1 | 1 | Reduced sensory disturbances |
| 006 | 3 | 3 | Enhanced emotional tone, better control |
| | | | of BP |
| 007 | 4 | 4 | Positive changes expressed as a |
| | | | moderate gain of strength in the |
| | | | paralysed extremities |
| 008 | 3 | 3 | No notable changes |
| 009 | 4 | 4 | No changes |
| 010 | 3 | 3 | Significant improvement of cognitive |
| | | | functions |

The disability assessment indicated a decrease in the mRS score (lower severity of disability) and better ability to attend own need (associated with reduced spastic paralysis in the arm, as judged by the investigator) for one of the ten patients (10%). Another patient exhibited reduction of sensory disturbances in the affected extremities. Five patients (50%) reported improvement of general health, as exhibited by enhanced mood, resolution of headaches, increased exercise tolerance and better memory. Three patients (30%) did not report any changes.

In this study AP drug was shown to have a beneficial effect on emotional health (anxiolytic, emotion-stabilizing action), as evidenced by improved general health, cessation of headaches, and enhanced mood in study patients after 28 days of treatment.

There were also beneficial effects observed in patients' sensation and paralysis severity and by improvement of cognitive functions, which was likely due to the drug's nootropic effect. These improvements are comparable to the effects of current rehabilitation interventions, such as therapeutic exercise, and muscle-relaxing agents. No side effects were produced by AP drug.

### Example 8

A study was conducted to examine the efficacy and safety of the claimed drug, formulated as a solid dosage form containing lactose granules impregnated with water-alcohol solution of the liquid activated (potentized), energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a difference of potentials being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug, where it was used for treating a patient diagnosed with ‘headache, mixed: common migraine, cervicogenic headache, tension-type headache’.

Patient V., a woman of 38 years, sought neurological consultation for increased attacks of unilateral throbbing headache, and also diffuse, band-like headache, with increased duration of attacks.

The patient had long been receiving neurologist care for ‘headache, mixed: common migraine, cervicogenic headache, tension-type headache’. To stop the attacks, she had been using triptan medicines (the effective one is Imigran), and occasionally ibuprofen; as a prevention, she had undergone vascular interventions, though with no significant effect achieved. She had also been prescribed anticonvulsants (topiramate), and antidepressants (paroxetine) to prevent the attacks, but the drugs had been stopped due to the development of side effects. At the time of the study, the frequency of migraine attacks experienced by the patient was 4 times a month (Visual Analogue Scale (VAS) score of up to 8), and episodes of non-migraine headache were reported to occur almost daily. The severity score of non-migraine headaches was up to 6. The patient was emotionally labile, easily prone to crying and had sleep problems: she reported difficulty getting to sleep and frequent waking up at night. The sustainable blood pressure was 110/70 mm Hg; the headache attacks were not accompanied by BP increases.

The patient was prescribed to take AP drug at 2 tablets three times daily for 3 months. Following the drug treatment, the frequency of all headache attacks was sufficiently reduced, with no side effects developed.

### Example 9

In a randomized, double-blind, placebo-controlled clinical study, claimed drug, formulated as a solid dosage form containing lactose granules impregnated with water-alcohol solution of the liquid activated (potentized), energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a difference of potentials being applied to electrodes placed in the initial neutral carrier and which is proportional in amplitude to an amplified combined, summed up signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug—was examine for efficacy and safety in treating anxiety in patients with neurotic (ND) and pseudoneurotic disorders (PND).

The trial included two parallel groups of patients aged between 18 and 75 years: 32 patients in the group receiving AP drug and 30 in the placebo group. The duration of study was 8 weeks (of which 4 weeks were drug administration) for both groups. The test drug was administered in the first group at 1 tablet 4 times daily for 4 weeks; the second group received placebo at 1 tablet 4 times daily for 4 weeks. Throughout the study, 5 scheduled visits were completed by patients in each of the study groups: 1 baseline visit (screening, randomization, dispensing of study drugs), 3 visits made for interim and final assessment (following 1, 2 and 4 weeks of treatment), and 1 follow-up visit made at 4 weeks after the completion of treatment.

The inclusion criterion was: anxiety of clinically significant severity (the total score on the Hamilton Anxiety Rating Scale (HAM-A) of ≧20, and the total score on the depression subscale of the Hospital Anxiety and Depression Scale (HADS)≧11. Concomitantly with the treatment administered the patients were permitted to take medications for the underlying disease, excepting anxiolytic agents. The dosage and dosing frequency for the concomitant medicines were kept, where possible, unchanged throughout the study. In cases of severe insomnia, short lived hypnotics were administered—standard doses of zolpidem or zopiclone. Where possible, the use of concomitant medications was minimized.

Efficacy Endpoints:

1. Mean reduction of anxiety symptoms as rated on the HADS and HAM-A following 1, 2, and 4 weeks of treatment and at 4 weeks after treatment termination.

For the safety assessment, the occurrence of side effects and their relatedness to the test drug were evaluated.

All patients enrolled had clinically significant symptoms of anxiety, as evidence by high HAM-A and HADS scores. The mean total anxiety scores in patient groups as measured by HAM-A were 27.28±0.66 in the group of AP drug and 26.37±0.6 in group of placebo; the total scores on the HADS' anxiety subscale were 14.75±0.46 vs. 15.7±0.41, respectively.

Thus, anxiety assessments were similar between the patient groups, both as evaluated for the overall patient population and specifically for patients with neurotic disorders compared to those with psudoneurotic ones (Table 16).

TABLE 16
Measurement values as obtained for the total patient groups and different patient subcategories

| | AP | | | PLACEBO | | |
|---|---|---|---|---|---|---|
| | Group's | | | Group’s | | |
| | overall | ND patients | PND patients | overall | ND patients | PND patients |
| | (n = 32) | (n = 16) | (n = 16) | (n = 30) | (n = 15) | (n = 15) |
| Score | (M ± m) | (M ± m) | (M ± m) | (M ± m) | (M ± m) | (M ± m) |
| Mean total | 27.28 + 0.66 | 26.38 + 0.82 | 28.19 + 1.01 | 26.37 + 0.6 | 27.13 + 0.65 | 25.6 + 0.9 |
| HAM-A score | | | | | | |

Total HADS’

| | | | | | | |
|---|---|---|---|---|---|---|
| anxiety score | 14.75 + 0.46 | 15.5 + 0.68 | 14.24 + 0.63 | 15.7 + 0.41 | 16.93 + 0.34 | 14.47 + 0.59 |
| depression score | 10.75 + 0.44 | 9.75 + 0.56 | 11.35 + 0.61 | 11.03 + 0.47 | 11.6 + 0.47 | 10.47 + 0.81 |

Analysis of anxiolytic effects in comparison between patients with ND and PND (Table 17) showed that the anxiolytic efficacy was somewhat higher in the former patient subcategory than that in the PND patients as evidenced by a greater reduction of the HAM-A score.

TABLE 17
Total HAM-A scores obtained at different study time points in patients with ND and PND

| | Patients subcategory | | | | | |
|---|---|---|---|---|---|---|
| | AP | | | PLACEBO | | |
| | | Treatment | Completion | | Treatment | Completion |
| | Baseline | termination | of follow-up | Baseline | termination | of follow-up |
| ND | 26.38 ± 0.82 | 16.4 ± 1.21 | 15 ± 1.01 | 27.13 ± 0.65 | 26.07 ± 1.05 | 26.78 ± 0.64 |
| PND | 28.19 ± 1.01 | 13.19 ± 0.7 | 13.31 ± 0.9 | 25.6 ± 0.9 | 19.6 ± 1.41 | 21.2 ± 1.31 |

The drug's anxiolytic efficacy was also indicated by changes in patients' HADS (Table 18) values following study treatment, as observed by a statistically significant reduction of anxiety ratings (HADS' Anxiety subscale) both by the end of treatment and 4 weeks after drug discontinuation, as compared to placebo. Comparison of the anxiolytic effect between patients with ND and PND (Table 18) showed that, similarly to the HAM-A outcomes, this was somewhat stronger in the ND patients than the effect in the PND subcategory as evidenced by a greater reduction of the HADS' anxiety score.

TABLE 18
Total HADS scores obtained at different study time points in patients with ND and PND

| Patients subcategory | | | | | |
|---|---|---|---|---|---|
| AP | | | PLACEBO | | |
| | Treatment | Completion | | Treatment | Completion |
| Baseline | termination | of follow-up | Baseline | termination | of follow-up |

Anxiety subscale, score

| | | | | | | |
|---|---|---|---|---|---|---|
| ND | 15.5 ± 0.68 | 8.8 ± 0.97 | 8.8 ± 0.74 | 16.93 ± 0.34 | 16.0 ± 0.53 | 15.6 ± 0.36 |
| PND | 14.24 ± 0.63 | 6.75 ± 0.39 | 6.5 ± 0.52 | 14.47 ± 0.59 | 9.87 ± 1.0 | 10.6 ± 0.97 |

### Efficacy Conclusions

AP exerts a marked anxiolytic effect in patients with neurotic and pseudoneurotic disorders following 4 weeks' treatment at 1 tablet 4 times daily, There were statistically significant improvements in the following endpoints as a result of the drug: reduction of the total HAM-A by 45.63%; a high percentage (41.3%) of patients whose total HAM-A was reduced by more than 50%; a reduction of 47.53% in the HADS' total anxiety score.

The anxiolytic effect of AP treatment was maintained after 4 weeks of drug discontinuation, both as observed for the total study groups and the different patient subcategories (ND and PND).

The achieved anxiolytic efficacy was somewhat stronger in patients with ND than in the PND subjects, being, though, significantly increased in both patient subcategories as compared to placebo.

The anxiolytic efficacy of AP demonstrated in this study is comparable to that of routine anxiolytic therapies (diazepam, clonazepam, phenazepam), as shown in previous clinical trials.

Similarly to the placebo, the drug was well tolerated by the patients throughout the observation period: adverse events were recorded for 1 patient in the AP (heartburn, eructation) and 1 patient in the placebo group (heartburn, autonomic disturbances). All patients in the study groups completed the treatment at the time specified by the study protocol; there were no early withdrawals. AP was not associated with any side effects.

### Example 10

A study was conducted to examine the efficacy and safety of the claimed drug, formulated as a solid (tablet) dosage form containing lactose granules impregnated with water-alcohol solution of the liquid activated (potentized), energy-treated carrier, obtained by multiple consecutivel dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a difference of potentials being applied to electrodes placed in the initial neutral carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug, where it was used for treating speech defects in children.

The efficacy and safety study of AP drug in the care for speech defective children was facilitated by a specialized school for children with speech problems. The study included 10 children of either sex (6 boys and 4 girls) aged from 7 to 8 years (primary school forms 1 to 2), who were diagnosed with speech defects defined by the ICD-10 as ‘specific developmental disorders of speech and language’ (F80), including: ‘specific speech articulation disorder’ (F80.0); ‘expressive language disorder’ (F80.1); ‘receptive language disorder’ (F80.2); ‘other developmental disorders of speech and language’ (F80.8). All children were prescribed to receive treatment with AP drug, at 1 tablet 3 times in the daytime for 12 weeks.

Prior to enrollment in the study, the children underwent diagnostic testing for speech problems as designed by T. A. Fotekova, T. V. Akhutina, 2002. The speech and language screening involves a set of tests that measure the development of different language components and consists of two sections.

Section 1: evaluation of expressive language development—involves 5 test sessions that assess:

1. Level of sensorimotor processes involved in speech and language: Processing of phonemic input; Articulation motor skills; Sound utterance; Reproduction of sound-syllabic structure of words.

2. Active vocabulary.

3. Word building skills.

4. Grammatical speech output.

5. Quality of continuous speech output.

Section 2: evaluation of impressive language development—involves 2 test sessions that assess:

1. Comprehension of word meaning

2. Comprehension of logical and grammatical structures.

The study included children whose average performance across the test sessions was 50% to 79.9%, i.e. performance grades III (79.9-65%) and II (64.9-50%).

Efficacy Endpoints:

Primary endpoint: Assessment of test performance in study groups after 12 weeks of treatment compared to baseline measurements; mean performance percentage on the speech/language tests in study groups after 12 weeks of treatment compared to baseline.

Secondary Endpoints:

Percentage of children with improved performance (increased performance outcomes (depending on quality rates)—a move from grade II (64.9-50%) to grade III (79.9-65%) or from grade III to grade IV (100-80%);

Safety Endpoints:

Occurrence and nature of adverse events during treatment and causal relationship with the study drug.

Prohibited Concomitant Medications:

The study participants were prohibited from using the following medicines for one month prior to enrollment in the study and throughout the treatment: Psychoanaleptics (ATC code N06), including psychostimulants and nootropics.

Study Results:

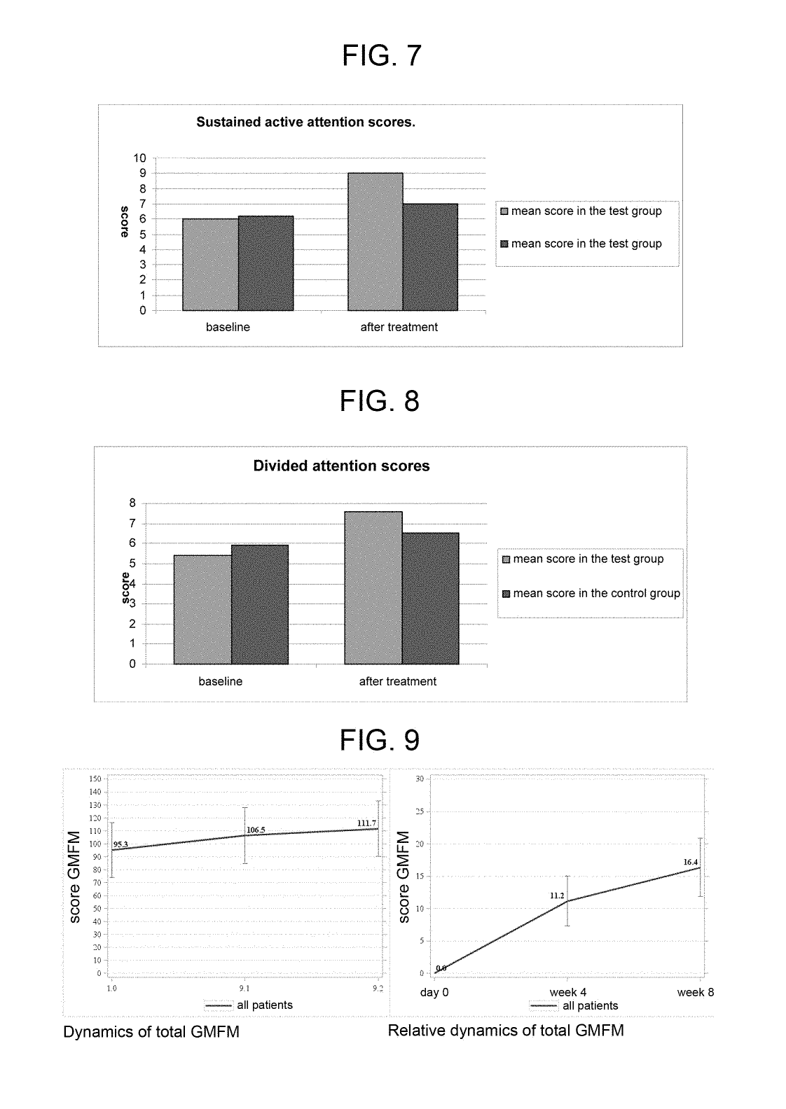

The evaluation results of the drug's efficacy in children with different developmental speech and language disorders are shown in FIGS. 7 and 8.

FIG. 7 displays changes in speech performance of a child aged 7 years and 8 months that mostly relate to impairments of speech perception, articulation motor skills and sound utterance, with normal levels achieved in vocabulary, grammatical speech output and comprehension of grammatical structures. As a result of study treatment, the children's articulation and sound utterance skills were almost within normal ranges, with significant improvements in the subjects' ability to reproduce word's sound-syllabic structure. In total, the resulting performance percentage demonstrated high statistical significance for nearly all tests involved in the assessment.

FIG. 8 presents a clinical case of a patient aged 8.5 years with predominantly deficient comprehension of syllabic word structure and deficit in language analysis skills, with deteriorating effects on grammatical speech output and vocabulary size. Following 12 weeks of treatment with AP drug, the patient demonstrated an improvement, with initially low measures of expressive language output (syllabic word structure, language analysis) observed to reach normal ranges, resulting in a better active speech quality.

Tables 19 and 20 present the percentages of performance demonstrated by study subjects at different time points of the study as well as changes in performance grades obtained for the total group of children examined (N=10).

TABLE 19
Performance percentage scores of children
undergoing speech/language assessment.
Mean performance percentages obtained
on the speech/language tests

| Patient | Visit 1 | Visit 2 |
|---|---|---|
| SS_120901 | 47.2% | 63.3% |
| SS_120902 | 76.7% | 89.9% |
| SS_120903 | 62.1% | 82.1% |
| SS_120904 | 47.6% | 63.7% |
| SS_120905 | 73.4% | 89.6% |
| SS_120906 | 61.1% | 78.2% |
| SS_120907 | 46.6% | 61.6% |
| SS_120908 | 70.3% | 86.8% |
| SS_120909 | 62.6% | 77.8% |
| SS_120910 | 79.7% | 91.2% |

Repeated measures ANOVA
Type 3 Tests of Fixed Effects

| Effect | Num DF | Den DF | F Value | Pr >F |
|---|---|---|---|---|
| Visit | 1 | 9 | 469.54 | <.0001 |

Descriptive statistics

| Visit | N Obs | Mean | Stand. Dev. |
|---|---|---|---|
| Visit 1 | 10 | 62.74% | 12.42 |
| Visit 2 | 10 | 78.41% | 11.71 |

TABLE 20
Performance grades of children undergoing speech/language assessment.
Changes in performance grades as assigned in speech/language testing

| | Performance grade, | Performance grade, |
|---|---|---|
| Patient | visit 1 | visit 2 |
| SS_120901 | I | II |
| SS_120902 | III | IV |
| SS_120903 | II | IV |
| SS_120904 | I | II |
| SS_120905 | III | IV |
| SS_120906 | II | III |
| SS_120907 | I | II |
| SS_120908 | III | IV |
| SS_120909 | II | III |
| SS_120910 | III | IV |

Thus, AP drug improves the measures of articulation motor skills and sound utterance and demonstrates efficacy in developing correct grammar of continuous speech output and expanding the vocabulary in children with different patterns of developmental speech disorders.

Following the course of AP drug, the percentages and grades of performance on speech development tests were improved (with statistical significance of changes obtained), as observed already after 12 weeks of treatment, favorably influencing the learning and social adjustment of children in the school facility. No adverse side effects due to the study drug were identified; it was well tolerated by study subjects over the whole length of treatment.

### Example 11

A randomized, comparative study was conducted to examine the efficacy and safety of the claimed drug, formulated as a solid (tablet) dosage form containing lactose granules impregnated with water-alcohol solution of the liquid activated (potentized), energy-treated carrier, obtained multiple consecutive dilution of the initial matrix solution in an aqueous-alcoholic diluent, combined with external mechanical impact—shaking of each resulting dilution, to a degree of 100200, equivalent to a centesimal (C200) homeopathic dilution (wherein the energy treatment of the initial neutral carrier has been accomplished by exposing it to an electric current caused by a difference of potentials being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug, where it was used for treating cognitive impairments (active attention, mnestic functions) in children with delayed mental development.

All patients were prohibited from taking psychoactive drugs at least a week prior to study initiation and throughout the study period. The observations involved 30 children (17 boys and 13 girls; age range, 5 to 8 years) with mental development delays (borderline states) who were diagnosed with: organic emotionally labile (asthenic) disorder with mild cognitive impairments; partial delays in higher-order mental functions (HMF) development with mild cognitive impairments; mood (affective) disturbances with emotional lability in attention deficit hyperactivity disorder; asthenoneurotic disorder of increasing severity; adjustment disorder with autonomic and psychosomatic reactions.

AP drug was administered at 1 tablet 3 times daily for 8 weeks.

The control group included 20 children (13 boys and 7 girls) with similar clinical features, who did not receive any specific drug treatment.

The results of psychological examination of cognitive functions were evaluated by levels of sustained and divided attention as well as mean scores derived from the Coding subset of Wechsler′ battery, as observed over time. At baseline, such mean scores of children in the test and control groups were equal to 6 (the score reflecting normality is 10).

In the group of test treatment, the mean score for sustained active attention rose from 6 to 9 after already 8 weeks of treatment, indicating improved focused attention. The relevant changes in the control group were less pronounced: the increase in the level of sustained active attention was from 6.3 to 7. The mean score for divided attention, as derived from this testing technique, increased in the test group from 6 (at baseline) to 7.5 (at treatment completion). In the second compared group, this parameter was considerably lower—6 at the start of treatment vs. 6.5 at study completion.

Since neuropsychological examination is often indicative of mnestic impairments in children with delayed psychological development, it is worthwhile to assess drug treatments for effects on memory. In the group of AP drug, examination of mnestic functions (scores for auditory-verbal memory and visual memory were derived a neuropsychological technique ‘Luria-90’ by E. G. Simernitskaya) yielded most pronounced improvements in the domains of auditory-verbal and visual memory and in some characteristics of visual memory: span, reproduction of a sequence of visual stimuli, and interhemispheric transfer of visual information. In the test group, the mean score for auditory-verbal memory was heightened from 5.8 to 8.1 and the increase of the visual memory score was from 6.2 to 7.9. In the control group, a minor increasing trend was noted in the measures of mnestic functions, these changes, though, being less significant than in the test group: the mean scores of auditory-verbal memory and visual memory were increased from 6.3 to 7.1 and from 6 to 6.7, respectively.

Conclusion. AP drug used in children with delayed psychological development improves cognitive functions (sustained and dividied attention), and enhances mnestic functions (auditory-verbal and visual memory)/The children's attention and memory were sufficiently improved after already 8 weeks of the drug course, as measured objectively by psychological tests and subjectively (by the parents' assessment), with beneficial effects on the subjects' learning and adjustment in a school facility. Noteworthy, none of the children developed adverse side effects due to the study drug, and it was well tolerated by the subjects throughout the treatment.

### Example 12

The study was conducted to evaluate the anxiolytic effect of the claimed drug, formulated as a water solution (liquid dosage form) of the activated (potentized) energy-treated carrier, obtained by consecutive multiple dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a difference of potentials being applied to electrodes placed in the initial neutral carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug, using standard experimental approaches—by testing study animals' open field behavior and behavior in the light/dark transition task. Twenty male albino Wistar rats were used for the experiments.

Ten rats were administered AP via the intragastric route at 1 ml/kg daily (the solution to be administered was diluted with purified water to a volume of 5 ml/kg) for 7 days. The other ten rats were used as controls and were administered activated (potentized) distilled water prepared using the homeopathic methodology as described above, at 5 ml/kg intragastrically, for 7 days.

The rats' behaviour reactions were examined by the above tests before administering AP drug or activated (potentized) distilled water (baseline data) and the day after the last intragastric dose of AP drug or activated (potentized) water (data at 8 days).

The animals' locomotor and exploratory activity was evaluated using the open-field apparatus. The latter is a round arena (98 cm in diameter, with 31 cm high walls) delineated into 4 central and 28 peripheral square sectors. The arena floor has 25 holes, each 3 cm in diameter. The apparatus is illuminated from above with a 100 W lamp. For the first 5 minutes, the animals were monitored for latency to enter the central sectors, the number of central squares crossed and the number of rears in the peripheral zone of the arena.

The light/dark transition test examined the animals' behavior under alternating stress conditions and exploratory motivation/anxiety balance. The apparatus consists of two compartments—a dark chamber and a brightly illuminated chamber of equal size (22 cm*44 cm*30 cm). The chambers are separated by a partition with a central door (10 cm high and 9 cm wide), through which test animals can move freely between the two compartments. The animals were monitored for 5 minutes to evaluate: the latency time to first enter the dark compartments, number of moves between the compartments, time spent by rats in each compartment, number of peeps through the door when on the dark side, and number of rears.

The obtained results were expressed as the mean (M) and standard error of the mean (SEM). Between-group statistical analysis was performed using the Mann-Whitney U test. The differences in data obtained at 8 days versus baseline were analyzed for statistical significance with the Wilcoxon test. The differences were considered significant at p<0.05.

AP drug was shown to reduce rats' latency time to enter the central sectors as compared to baseline and control values—by 26.9% (p<0.05) and 19.8% (p<0.05), respectively. The number of peripheral squares crossed was reduced by 34.1% (p<0.05) vs. baseline and by 53.4% (p<0.05) vs. control. At the same time, the number of central squares crossed was increased by 100% (p<0.05) and 40% (p<0.05) as compared with the baseline and control data, respectively. The number of rears at the periphery of the arena was reduced by 42.7% (p<0.05) compared to baseline (Table 21).

TABLE 21
Open field behaviour of male rats administered AP drug

| | | Control | AP |
|---|---|---|---|
| Variable | Conditions | (M ± SEM) | (M ± SEM) |
| Latency time to | Baseline | 289.2 ± 10.8 | 250.5 ± 13.29 |
| enter the central | Day 8 | 228.1 ± 7.27* | 183.0 ± 3.90*^{#} |
| zone, s | | | |
| Number of | Baseline | 42.1 ± 6.84 | 26.7 ± 7.59 |
| peripheral squares | Day 8 | 29.2 ± 7.94 | 13.6 ± 5.22*^{#} |
| crossed, n | | | |
| Number of central | Baseline | 1.0 ± 0.67 | 0.7 ± 0.47 |
| squares crossed, n | Day 8 | 1.0 ± 0.54 | 1.4 ± 0.73*^{#} |
| Number of rears | Baseline | 9.3 ± 1.67 | 7.5 ± 1.90 |
| at the arena | Day 8 | 4.6 ± 1.14* | 4.3 ± 1.21* |
| periphery, n | | | |

Note:
*p < 0.05—significant differences vs. baseline (within-group comparisons);
^{#}p < 0.05—significant differences vs. control (Day 8).

In the light/dark transition task, rats' latency to enter the dark compartment was lengthened by 76.8% (p<0.05) compared to baseline. On the contrary, the number of moves between the compartments was reduced by 58.8% (p<0.05) compared to baseline. In the group of AP drug, the time spent by test animals in the light compartment was found to increase by 76.9% (p<0.05) and 52.9% (p<0.05) compared to baseline and control values, respectively. The time spent in the dark compartment was reduced by 15.3% (p<0.05) compared to baseline. The reductions in the number of rears vs. baseline and control were 30.9% (p<0.05) and 37.4% (p<0.05), respectively (Table 22).

TABLE 22
Light/dark transition behaviour of male rats administered AP drug

| | | Control | | AP | |
|---|---|---|---|---|---|
| Parameter | Conditions | (M ± SEM) | | (M ± SEM) | |
| Latency time to first | Baseline | 18.4 ± | 4.8 | 6.9 ± | 2.28 |
| enter the dark | Day 8 | 11.5 ± | 2.99 | 12.2 ± | 3.62* |
| compartment, s | | | | | |
| Number of moves | Baseline | 2.8 ± | 0.66 | 2.67 ± | 0.59 |
| between the | Day 8 | 3.2 ± | 0.59 | 1.1 ± | 0.25* |
| compartments, s | | | | | |
| Time spent in the light | Baseline | 51.3 ± | 21.73 | 26.8 ± | 6.32 |
| compartment, s | Day 8 | 31.0 ± | 8.32 | 47.4 ± | 30.24*^{#} |
| Time spent in the dark | Baseline | 248.6 ± | 21.75 | 289.8 ± | 5.91 |
| compartment, s | Day 8 | 242.1 ± | 25.34 | 245.6 ± | 30.23* |
| Number of peeps | Baseline | 5.4 ± | 1.73 | 6.2 ± | 1.10 |
| from the dark | Day 8 | 6.3 ± | 1.76 | 4.8 ± | 1.23 |
| compartment, n | | | | | |
| Number of rears, n | Baseline | 11.7 ± | 2.77 | 9.7 ± | 1.51 |
| | Day 8 | 10.7 ± | 2.16 | 6.7 ± | 1.60*^{#} |

Note:
*p < 0.05 as compared to baseline (‘before/after’ within-group comparisons).

Thus, AP produces a marked anxiolytic effect.

### Example 13

A study was conducted to evaluate the nootropic effect of the claimed drug, formulated as a water solution (liquid dosage form) of the activated (potentized) energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial liquid carrier has been accomplished by exposing it to an electric current caused by a difference of potentials being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug, using standard experimental approaches—by examining the test drug's antiamnestic effects in scopolamine-induced amnesia for passive avoidance conditioning in sexually mature rats. A total of 36 male albino Wistar rats were used for the study.

For amnesia induction, 15 minutes prior to passive avoidance training, the animals were administered a single intraperitoneal dose (2 mg/kg) of a muscarinic receptor antagonist—scopolamine.

AP drug was administered to 12 rats intragastrically, at 2.5 ml/kg daily, prior to training (1 hour before the start of the experiment), immediately after training, 24 hours post training, and 1 hour prior to the passive avoidance recall test. Twelve rats in the control group received intragastric doses (25 ml/kg daily) of activated (potentized) distilled water prepared using the above-mentioned homeopathic methodology, on the same schedule as described for the AP group. The reference compound—piracetam (nootropil) was administered using the same schedule, at 400 ml/kg intragastric doses.

On the memory recall test, the rats' behavior was evaluated by examining: latency time taken by test animals to first enter the dark compartment and total time spent therein over a certain time period.

The conditioning of passive avoidance behavior was as follows: On day 1, the animals were trained to stay in a shuttle box, which consisted of a large, 300×300×250 mm, white illuminated compartment (with transparent walls) and a smaller, 150×150×180 mm, black dark compartment with an electrical grid floor. On the second day, the rats were placed in the middle of the white compartment, tails towards the guillotine gate separating the compartments. On finding the gate, the test animal moved to the black compartment. For 3 minutes, the total times of the rat's stay in the small compartment and latency before moving in were recorded. After the 3-minute interval, while remaining in the black compartment, the animal received an electric shock of 0.2 mA from the grid floor, which was maintained until the rat escaped to the white compartment. If the rat did not return to the black compartment within 10 seconds, it was removed from the box and placed back in the home cage. If the animal entered the compartment again, it was exposed to another series of electric shock. The retention of passive avoidance memory was tested 24 hours after the acquisition phase by placing the test rat in the white compartment and observing its latency to enter the black one. The time before moving to the black compartment is longer with a better recall of the electric shock previously received when in the dark compartment.

The data gathered were recorded as the mean (M) and standard error of the mean (SEM). Between-group statistical analysis was performed using the Mann-Whitney U test. The results of examining the rats' memory recall were analyzed for statistical significance with the Wilcoxon test. The differences were considered significant at p<0.05.

AP drug was shown to significantly increase rats' latency to enter the ‘dangerous’ compartment—by 69% as compared to controls and 87% compared to baseline. The animals' length of stay in the dark compartment was reduced by 23% (p<0.05) compared to the control value and 47% (p<0.05) compared to that at baseline (Table 23).

TABLE 23
Behavior variables of male rats receiving AP drug in the assessment
of its antiamnestic effects in scopolamine-induced amnesia

| | | Control | | Piracetam | | AP | |
|---|---|---|---|---|---|---|---|
| Parameter | | (M ± SEM) | | (M ± SEM) | | (M ± SEM) | |
| Latency to enter the | Training | 8.78 ± | 1.86 | 11.07 ± | 2.02 | 6.57 ± | 1.55 |
| aversive compartment, | Memory | 16.13 ± | 2.84 | 53.71 ± | 3.65*& | 52.38 ± | 7.43*& |
| s | recall | | | | | | |
| Time spent in the black | Training | 27.80 ± | 5.03 | 21.1 ± | 1.62 | 28.87 ± | 1.93 |
| compartment, s | Memory | 20.04 ± | 2.65 | 13.11 ± | 0.76*& | 15.34 ± | 2.68*& |
| | recall | | | | | | |

Note:
*p < 0.05 as compared to control;
& - p < 0.05 compared to baseline (training)

Thus, the claimed drug has a marked nootropic effect.

### Example 14

A study was conducted to evaluate the antidepressant effect of the claimed drug, formulated as a water solution (liquid dosage form) of the activated (potentized) energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a difference of potentials being applied to electrodes placed in the initial liquid carrier and which is proportional in amplitude to an amplified combined, summed up signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug, using standard experimental approaches—by examining animals' depression-like behavior (behavioral despair) in the Porsolt forced swimming test and Nomura's model employing forced-rotating-wheel swim test. Male albino Wistar rats (weight range, 250-300 g) were used for the experiments.

Antidepressant properties of test compounds were studied after single-dose administration in experimental animals. For this, the animals in each study group were given single intragastric doses of either 2.5 ml/kg of AP drug or 2.5 ml/kg of activated (potentized) distilled water (control), prepared using the homeopathic methodology as described above. The reference compound (amitriptyline) was administered to test rats at 10 mg/kg in the amount of 2.5 ml/kg.

The Porsolt behavioral despair (hopelessness) test involves placing test animals in a container filled with water, where they begin to demonstrate vigorous struggling behavior directed at escaping the aversive stimulus, followed after a while by giving up making any movements beyond those required to keep their heads above water. The length of immobility (immobility time) is the measure of depressive state.

In Nomura's variant of this behavioral test, the locomotion response of test animals, placed in a water tank, is measured by the number of rotations of water wheels located in 4 tank sections. The number of wheel rotations indicates the absence of anxiety-like (depressive) state in test animals. Wheel rotations were measured for 10 minutes.

All test results were expressed as percentages of the control values obtained in the rat group receiving potentized water. Between-group statistical analysis was performed using the Mann-Whitney U test. The differences were considered significant at p<0.05.

Based on the results of the Porsolt behavioral despair (hopelessness) test, the AP test compound exhibited marked antidepressant effects: the immobility time of AP-treated animals was reduced by 42% compared to controls (p<0.05). This variable obtained for the reference compound was 56% (p<0.05).

Similarly, marked antidepressant effects of the AP test compound were evidenced by the results of Nomura's modification of the despair swim test: the number of wheel rotations was increased by 63% compared to controls (p<0.05). This variable of the reference compound was 67% (p<0.05).sjhn

Thus, the claimed drug has a marked antidepressant effect.

### Example 15

A study was conducted to evaluate the anticonvulsant effect of the claimed drug, formulated as a water solution (liquid dosage form) of the activated (potentized) energy-treated carrier, obtained by extreme serial dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial liquid carrier has been accomplished by exposing it to an electric current caused by a difference of potentials being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug, using standard experimental approaches—by scoring the test drug's anticonvulsant efficacy according to Racine's system: (Racine 1972; Scarletti-Lima et al., 2003)—on a scale from 1 to 10 after a single 60 mg/kg dose of pentylenetetrazol administered to test animals. A total of 14 male albino Wistar rats were used for the experiment.

AP drug was administered intragastrically, at 10 ml/kg daily for 7 days (n=7). Animals in the group of control compound (n=7) received activated (potentized) distilled water (control), prepared using the above-mentioned homeopathic methodology, in intragastric doses of 10 ml/kg daily for 7 days.

The severity of seizures and latent time from pentylenetetrazol administration to seizure development were evaluated the day after the last dose of test compounds (data on day 8).

The evaluation results were expressed as the mean (M) and standard error of the mean (SEM). Between-group statistical analysis was performed using the Student's t-test. The differences were considered significant at p<0.05.

As demonstrated by the results, AP drug reduces the severity of seizures 30% (p=0.01) more effectively than the control compound, with the latent time of seizure development increased by 57% (p=0.03) compared to the control value (Table 24).

TABLE 24
AP drug's anticonvulsant effects as scored on the Racine scale

| | AP | | Control | |
|---|---|---|---|---|
| Parameter | (M ± SEM) | | (M ± SEM) | |
| Max convulsion severity, score | 3.6 ± | 0.4* | 5.1 ± | 0.3 |
| Latency time, s | 141.0 ± | 29.6* | 60.6 ± | 2.7 |

*p < 0.05 as compared to control

Thus, AP drug has a marked anticonvulsant effect.

### Example 16

A study was conducted to evaluate the neuroleptic effect of the claimed drug, formulated as a water solution (liquid dosage form) of the activated (potentized) energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a potential difference being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug, using standard experimental approaches—by examining experimental animals' behavior in the startle response/prepulse inhibition test. A total of 60 male albino Wistar rats were used for the experiment.

The approach utilized provides a ‘translational’ model of schizophrenia, represented by impaired prepulse inhibition (PPI) of acoustic startle response. PPI is the result of sensorimotor gating, and its deficit of which is the main cause of impaired selective attention in schizophrenic patients. Neuroleptics reverse PPI deficit. For the experiment, PPI deficit characteristic of schizophrenia was induced by ketamine. The works were performed with the use of male albino Wistar rats (n=60), 20 of which were administered intragastric doses of AP drug at 10 ml/kg daily for 5 days prior to the experiment (group 1). Rats in the active control (group 2, n=20) and passive control groups (group 3, n=20) received activated (potentized) distilled water prepared using the above-mentioned homeopathic methodology, in intragastric doses of 10 ml/kg daily for 5 days. Prior to the experiment, rats in groups 1 and 2 received subcutaneous injections of 10 mg/kg ketamine solution in water, and animals in group 3 were administered identical volumes of distilled water. One week before the main session, the animals underwent a preliminary test and were divided into treatment groups with similar evaluation results.

The acoustic startle effect in animal groups was estimated by an automated procedure on a modular system from Coulbourn Instruments (USA). The animals were placed in 8×15 cm holders mounted on load cell platforms with integrated piezoelectric accelerometers. The platforms were placed in a ventilated light-and sound attenuated chamber. 20 ms duration flashes of white noise were used as trials. Prepulse trials of three intensities were utilized: 73, 75 and 80 dB (pre-threshold values), with the pulse intensity of 120 dB (suprathreshold value). The test was conducted with the background white noise of 70 dB (white noise generator WNG 023, Russia).

The amplitude of acoustic startle response (ASR), reflecting the signaling effectiveness in the related neural network, was recorded on a microcomputer IBM PC/AT from peak voltages proportional to the accelerometer drift rate, over a 200 s interval from the start of pulse presentation and expressed in arbitrary units (‘startle response units’). The test was performed by presenting the rats (after a 5-minute acclimation period) with a set of 40 random-sequence acoustic signals of 4 types: startle pulse trial and 3 different combinations of a prepulse (73, 75, and 80 dB) followed by the pulse. The amount of response to combined trials (prepulse+pulse) was determined from amplitude Aᵢₙₜ. The ASR to the startle pulse was taken as 100%. Prepulse inhibition of ASR was calculated as PPI, %=100%−(Aᵢₙₜ−Aₚᵤₗₛₑ)*100%. The mean IIP was calculated for all same-type trials for each animal, after which group means were derived.

The data were recorded as the mean (M) and standard error of the mean (SEM). Between-group statistics were performed using the one-way analysis of variance (ANOVA) and by comparing sets of means by Duncan's test or LSD test. The differences were considered significant at p<0.05.

In the active control group, the PPI was significantly reduced as a result of ketamine (PPI₇₃: F_{(1.56)}=8.1; p<0.05; PPI₇₅: F_{(1.56)}=5.0; p<0.05; PPI₈₀: F_{(1.56)}=4.4; p<0.05; PPIₘₑₐₙ: F_{(1.56)}=7.6; p<0.05), indicating a deficit of sensorimotor gating in test animals. The PPI of test animals was significantly increased following AP drug compared to ketamine alone (group 2), as observed for prepulse intensities of 75 and 80 dB, with the same effect on the mean PPI (PPI₇₅: F_{(2.67)}=14.1; p<0.01; PPI₈₀: F_{(2.67)}=13.7; p<0.01; PPIₘₑₐₙ: F_{(2.67)}=15.6; p<0.01). In the group of AP drug and ketamine, the PPI value did not reach the control level for any signal intensity, exceeding PPI in the active control group, which might be associated with the initially high PPI in the passive control group as observed in this experiment (Table 25).

TABLE 25
Effect of AP drug on prepulse inhibition of acoustic startle
response in rats

| | | PPI, | |
|---|---|---|---|
| Group | PPI_{startle pulse, dB} | % ± SEM | |
| Group 1 (AP + ketamine) | PPI₇₃ | 28 ± | 4^{#} |
| | PPI₇₅ | 28 ± | 5^{#}* |
| | PPI₈₀ | 39 ± | 4^{#}* |
| | PPIₘₑₐₙ | 32 ± | 4^{#}* |
| Group 2 (water + ketamine) | PPI₇₃ | 19 ± | 5^{#} |
| | PPI₇₅ | 15 ± | 4^{#} |
| | PPI₈₀ | 24 ± | 5^{#} |
| | PPIₘₑₐₙ | 20 ± | 4^{#} |
| Group 3 (passive control - water) | PPI₇₃ | 39 ± | 4 |
| | PPI₇₅ | 44 ± | 4 |
| | PPI₈₀ | 50 ± | 4 |
| | PPIₘₑₐₙ | 45 ± | 4 |

*p < 0.05 from group 2 (water + ketamine);
^{#}p < 0.05 from group 3 (passive control − water);

Thus, the claimed drug has a marked neuroleptic effect.

### Example 17

B study was conducted to evaluate the neuroactive effect of the claimed drug, formulated as a water solution (liquid dosage form) of the activated (potentized) energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a potential difference being applied to electrodes placed in the initial neutral carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug, using standard experimental approaches—by examining experimental animals' behavior in the elevated plus maze test. A total of 21 male albino Wistar rats were used for the experiment, 11 of which received intragastric doses of AP drug, at 5 ml/kg daily for 7 days. Ten rats in the control group were dosed intragastrically with activated (potentized) distilled water prepared using the above-mentioned homeopathic methodology, at 5 ml/kg daily for 7 days.

The rats' behavior responses in the above test were examined before administering AP drug or activated (potentized) water (baseline data), and the day after the last dose of the test compounds (data on day 8).

The elevated plus maze apparatus consists of 4 arms, 0.5 m long and 10 cm wide, fastened at right angle to one another. Two opposite arms are enclosed on three sides with 40 cm height walls, with the other two open. The maze is elevated 0.5 m above the floor, with the entire runway area illuminated with 20 W fluorescent lamps positioned at 60 cm above the apparatus. Each rat was placed at the junction of the four arms, the tail towards the open one, and was observed visually for 3 minutes to measure the animal's latency to enter one of the arms, time spent in the open and enclosed arms, and number of head dips off the maze edges.

The measurement results were expressed as the mean (M) and standard error of the mean (SEM). Between-group statistics were performed using the Mann-Whitney U test. The data obtained at 8 days versus baseline were analyzed for statistical significance with the Wilcoxon test. The differences were considered significant at p<0.05.

As shown by the results, the animals' latency to leave the junction area was increased by 70.2% versus baseline and the time spent in the enclosed arms was lengthened by 7% (p<0.05) compared to the baseline following AP drug. The time spent in the open arms was reduced by 56.5% (p<0.05) and 66% (p<0.05), respectively, compared to baseline and control values (Table 26).

TABLE 26
Behavior variables of male rats in the elevated plus maze test following
repeat-dose exposure to AP drug

| | | Control | | AP | |
|---|---|---|---|---|---|
| Parameter | Conditions | (M ± SEM) | | (M ± SEM) | |
| Latency time to leave | baseline | 8.4 ± | 2.63 | 3.7 ± | 1.09 |
| the junction, s | Day 8 | 7.2 ± | 1.71 | 6.3 ± | 1.79 |
| Time spent in the | baseline | 123.5 ± | 11.95 | 136.0 ± | 7.83 |
| enclosed arms, s | Day 8 | 82.1 ± | 16.04* | 145.5 ± | 10.82* |
| Time spent in the | baseline | 27.3 ± | 7.46 | 29.2 ± | 6.78 |
| open arms, s | Day 8 | 37.5 ± | 9.77 | 12.7 ± | 6.58*^{#} |
| Number of head | baseline | 3.1 ± | 0.88 | 2.5 ± | 0.56 |
| dips, n | Day 8 | 8.1 ± | 1.35** | 4.3 ± | 1.54 |

Note:
*p < 0.05 as compared to controls;
**p < 0.01 as compared to baseline;
^{#}p < 0.05 as compared to control data on day 8.

Thus, the claimed drug has a marked sedative effect.

### Example 18

A study was conducted to evaluate the neuroactive effect of the claimed drug, formulated as a water solution (liquid dosage form) of the activated (potentized) energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial liquid carrier has been accomplished by exposing it to an electric current caused by a difference of potentials being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug, using a standard rat model of a photochemically induced focal lesion of the brain's prefrontal cortex. A total of 48 male albino Wistar rats were used for the experiment.

Prior to inducing an ischemic lesion in the rat brain, all experimental animals were given passive avoidance conditioning over a three-day period, which allowed for measuring post-ischemic memory impairments and determining memory retention following administration of the investigated neuroprotector drug. Passive avoidance conditioning is based on rats' natural preference for limited, enclosed space (hole exploratory behavior). On day 1, the animals were trained to stay in a shuttle box, which consisted of a large, 300×300×250 mm, white illuminated compartment (with transparent walls) and a smaller, 150×150×180 mm, black dark compartment with an electrical grid floor. On the second day, the rats were placed in the middle of the white compartment, tails towards the guillotine gate separating the compartments. On finding the gate, the test animal moved to the black compartment. For 3 minutes, the total times of the rat's stay in the small compartment and latency before moving in were recorded. After the 3-minute interval, while remaining in the black compartment, the animal received an electric shock of 0.2 mA from the grid floor, which was maintained until the rat escaped to the white compartment. If the rat did not return to the black compartment within 10 seconds, it was removed from the box and placed back in the home cage. If the animal entered the compartment again, it was exposed to another series of electric shock. The retention of passive avoidance memory was tested 24 hours after the acquisition phase by placing the test rat in the white compartment and observing its latency to enter the black one. The time before moving to the black compartment is longer with a better recall of the electric shock previously received when in the dark compartment.

To induce a focal ischemic brain lesion, anaesthetised rats (intraperitoneal 300 mg/kg chloral hydrate) were given injections of 3% rose bengal stain (40 ml/kg) in the jugular vein. The animals' head was firmly fixed in the stereotactic head-holder, following which the skin was cut along the sagittal suture and the pericranium separated. Light from a 250-W halogen lamp (24V) was carried via an optical fiber (3 cm, core diameter) to the skull area over the right and left frontal cortex. The exposure lasted for 15-20 minutes on either side. To prevent thermal coagulation, the light-exposed skull was chilled with tap water (added dropwise from a syringe, once in 2-3 minutes).

AP drug was administered to 12 rats intragastrically, at 5 ml/kg daily, over 5 days prior to photothrombosis induction (the last dose—1 hour before thrombosis development) and for the subsequent 9 days post-ischemia. Twelve rats in the control group received intragastric doses (25 ml/kg daily) of activated (potentized) distilled water, prepared using the above-mentioned homeopathic methodology, as described for the AP group. The reference compound—piracetam (nootropil) was administered as 400 ml/kg intragastric doses, following the above schedule. On the memory recall test, the rats' behavior was evaluated by examining latency time taken by test animals to first enter the dark compartment and total time spent therein over a certain time period. Twelve rats in the control group received intragastric doses (5 ml/kg daily) of activated (potentized) distilled water prepared using the above-mentioned homeopathic methodology, on the same schedule as described for the AP group. The last dose was administered 40 minutes before the start of the test. Sham-operated (n=12) and (n=12) intact rats were also used in the experiment as additional controls.

On post-ischemic day 4, 2 rats were randomly selected from each group and used to obtain brain specimens (2-3 hours after the last dose of test compounds) for morphological measurements. Before collecting brain specimens, the rats were sacrificed and their central nervous system was fixed in situ by transcardial perfusion (formalin, acetic acid and alcohol). On days 5 and 9, 1 hour after administration of AP drug or activated distilled water, the animals were examined in the passive avoidance test. On day 9, after examining the retention of passive avoidance memory, the rats were removed from the experiment for subsequent studies by an hour's anaesthesia with chloral hydrate.

To estimate the lesion area and cerebral ischemia volume, the test animals' brain was fixed by transcardial perfusion with 10% neutral buffered formalin. Then 100 μm thick serial vibratome sections were prepared, after which they were mounted on slides using 1% gelatin in 70° ethanol, stained with 0.2% methylene blue, dehydrated with increasing concentrations of alcohol, clarified in xylol and embedded in resin for use in subsequent measurements of ischemic lesion area on serial sections.

The data gathered were recorded as the mean (M) and standard error of the mean (SEM). Between-group statistics were performed using the Mann-Whitney U test (size of focal lesion) and two-way repeated measures ANOVA, followed by multiple comparisons by post-hoc Tukey's test (passive avoidance behavior). Differences were considered significant at p<0.05.

Statistical analysis of morphological measurements was performed using the conventional ImageJ software (Research Services Branch of the National Institute of Mental Health, USA).

As shown by the experiment results, AP drug provides memory retention in ischemic rats similar to that observed for intact and sham-operated animals (Table 27).

The size of the focal stroke lesion in rats administered AP drug was 1.935±0.144 mm³ versus 3.439±0.248 mm³ in control animals.

Thus, AP drug was shown to have a marked neuroprotector effect: it provides memory retention following stroke and reduces focal ischemic lesion volume in the brain.

### Example 19

An study was conducted to examine the efficacy of the claimed drug, formulated as a solid (tablet) dosage form containing lactose granules impregnated with water-alcohol solution of the liquid activated (potentized), energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial liquid carrier has been accomplished by exposing it to an electric current caused by a difference of potentials being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug—in the treatment of Parkinson's disease.

Patient K (woman, 73 years) had long been under neurologist care for a diagnosis of ‘Parkinson's disease mainly characterized by tremor and rigidity. Hoehn and Yahr stage: 2.5’.

Complaints: severe morning stiffness, problems getting to sleep, frequent awaking at night, nightmares involving screaming and fidgeting, constipations.

Several years earlier, the patient developed tremor, more severe in the left extremities, and began to feel stiffness in the same extremities a year later; the treatment prescribed was an L-DOPA medication at 300 mg daily providing good stiffness and tremor control. After some time, the problems reoccurred, for which the treatment was supplemented with amantadine, with the dose progressively increased to 400 mg daily. The therapy had no meaningful effect, and it was further extended by adding piribedil, at a maximum daily dose of 200 mg daily. Each new stage of treatment at first resulted in improvement in the patient's state; however, it was subsequently canceled out by reoccurring stiffness and tremor. Over the last year, the patient had experienced episodes of sudden rigidity (while continuing to take medications) accompanied by low mood and anxiety and followed by its equally sudden disappearance (‘on-off’ phenomenon), due to which it was recommended to divide the daily L-DOPA dose into 5 times. As a result, the severity of stiffness was reduced, but the rest of the problems were gradually aggravating.

On examination: Oligo- and bradymimia. Oily skin. BP, 110/70 mm Hg. No pareses. Oligo- and bradykinesia. Increased muscle tone, cogwheel muscle rigidity. No abnormal signs. No sensory disorders. Intention tremor on finger-to-nose test. Gait with small shuffling steps, propulsion. Pelvic functions are under voluntary control, nocturia (up to 6 urinations per night), constipations. Cognitive manifestations: oligo- and bradyphrenia. Insomnia with behavioral disturbances during REM sleep.

AP drug was administered as part of the treatment, at 2 tablets*3 times daily for 3 months.

After the course of the test drug, the patient reported apparent improvements in her state: resolution of episodic stiffness experienced previously between doses of routine antiparkinson medications, better mood, improved sleep (fewer wakes and urination urges during the night), reduced constipation severity. In addition, the patient and family members reported substantial improvement of memory and response time during conversations.

The severity of Parkinson's symptoms was evaluated using the UPDRS scale, with improvements observed on Part I (mentation) I—by 20% (score reduction), Part II (activities of daily life)—15%, Part III (motor evaluation)—20%, and Part IV (complications of therapy)—25%.

Thus, in the case described, AP drug demonstrated moderate antiparkinson efficacy in providing tremor and rigidity control (by enhancing the effect of routine antiparkinson therapies and eliminating the ‘on-off’ effect) and high efficacy in reducing co-existing symptoms (by producing hypnagogic, anxiolytic, enterokinetic, and nootropic-like effects).

### Example 20

A study was conducted to examine the efficacy of the claimed drug, formulated as a solid (tablet) dosage form containing lactose granules impregnated with water-alcohol solution of the liquid activated (potentized), energy-treated carrier obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a difference of potentials being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug—in the treatment of somatoform autonomic dysfunction.

Patient R (woman, 23 years);

### Diagnosis: Somatoform Autonomic Dysfunction

Complaints: episodic heart palpitations, non-exertional shooting pain of different durations at the apex of the heart, non-exertional and non-posture-related dyspnea, severe generalized weakness, easy fatigability.

These complaints had been experienced for a year. The onset had been due to emotional stress during the exam period.

On examination: Alert. Normal skin colour, no cyanosis or edema. BMI: 18. Lungs: vesicular breath sounds, no adventitious sounds. RR: 14 per minute. Heart boundaries within normal limits. Heart sounds unchanged. Extra heart sounds or murmurs are not heard. BP: 104/62 mm Hg, pulse: 74 per minute. Abdomen is soft, nontender. Liver edge is palpable at the costal margin. Kehr's, Ortner's, or Murphy's signs are negative. Easy urination. No dysuria. Urine output/24 h: 1000 ml. Stool: once/24 h, formed. Neurologic status: no abnormalities.

ECG: sinus rhythm, normal CEA. ECG: within normal limits. Clinical blood test: Hb −124 g/L; WBC 4.2×10⁹/L (eosinophils-2, stabs-4, segs-65, lymphocytes-23, monocytes-10). Total bilirubin 14.3 μmol/L; conjugated bilirubin—2.4; ALP—64 U/L; ALT—17.4 U/L; AST—23.1 U/L; GGT—6.0 U/L; cholesterol—4.1 mmol/L; LDH—136 U/L; CPK—92 U/L; glucose—3.8 mmol/L; hemodiastase—76 U/L; uric acid—224 μmol/L. Heart rate variability: autonomic function decline, predominantly parasympathetic. Reduced adaptive capacity.

The patient was prescribed to take AP drug at 2 tablets 3 times for a month. As a result of treatment, clinical variables demonstrated improvement. Chest pain and dyspnoea were resolved, with lesser asthenia and fatigability.

HRV measurements suggested an increase in autonomic function together with restoration of sympathetic/parasympathetic balance.

Treatment outcome: improvement.

Therefore, treatment with AP drug as described above has a beneficial effect on the autonomic nervous system.

### Example 21

A study was conducted to examine the efficacy of the claimed drug, formulated as a water solution (liquid dosage form) of the activated (potentized) energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial liquid carrier has been accomplished by exposing it to an electric current caused by a difference of potentials being applied to electrodes placed in the initial neutral carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug—in the treatment of phobias and anxiety disorders in children.

The study included 28 children (age range, 5 to 15 years) with a confirmed diagnosis of anxiety disorder as defined by the ICD-10 (‘emotional disorders with onset specific to childhood’ (F93): separation anxiety disorder of childhood (F93.0), phobic anxiety disorder of childhood (F93.1), social anxiety disorder of childhood (F93.2), generalized anxiety disorder of childhood F93.80); mild to severe.

The severity of anxiety disorders (from mild to severe) was determined using anxiety rating scales for children and adolescents designed by G. P. Lavrentieva, T. M. Titarenko. The overwhelming majority of children had severe (59%) or moderate (39%) anxiety disorder. AP was dosed at 10 drops 3 times daily for 12 weeks. As early as 4 weeks after treatment initiation (visit 2), the percentage of children with severe anxiety was reduced from 59% to 33%. By the end of the 12-weeks' treatment, the percentage of children with severe anxiety disorders was 20% and children with moderate anxiety were estimated to reach 78%, which evidences the test drug's anxiolytic-like activity.

Notably, the anxiolytic effects of AP drug were most pronounced in younger participants (5 to 7 years old), which can be relevant in treating pre-school age patients, who are not normally administered tranquilizers due their high addictiveness and often an opposite excitatory effect. A significant anxiety reduction was demonstrated by the children as early as visit 3 (at 8 weeks), with the effect steadily increasing through visit 4 (at 12 weeks). Mean anxiety scores among the 5-7-year-olds were reduced by 11.9 following 12 weeks of treatment.

AP drug had a beneficial effect on social phobia scores: in the age group of 8-15 years, phobic severity was reduced from 8.5±4.0 to 5.7±3.4 and, by the parents' rating, from 8.9±3.3 to 5.7±3.2. SCAS scores obtained in the 5-7-year-old patients indicated a clear anxiolytic-like effect of AP drug. The mean parents' rating on the same scale was reduced by 40% (5.5±3.9) as measured at treatment completion compared to baseline (9.6±5.5). In addition, as judged by the parents of 5-7-year-olds, the children's fear of an injury was reduced by the test drug: the score declined from 7.5±3.7 to 5.1±3.6.

Conclusion. As shown by the patients' self-assessments and parents' reports, AP drug is effective in 12-week treatment for anxiety disorders in children and adolescents. The drug exhibits a significant anxiolytic effect within the first week of treatment, with subsequent anxiety scores decreasing progressively throughout the observation period (12 weeks). AP drug was not associated with any side effects.

### Example 22

A study was conducted to examine the efficacy of the claimed drug, formulated as a water solution (liquid dosage form) of the activated (potentized) energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial liquid carrier has been accomplished by exposing it to an electric current caused by a difference of potentials being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug—in treating epilepsy accompanied by mild personality changes, multiform seizures.

Patient F (woman, 23 years old).

Diagnosis: ‘epilepsy with mild personality changes, multiform (tonic-clonic and absence) seizures’.

Based on medical history, Positive family history: maternal grandmother epileptic. Father epileptic (related to alcohol). Early development was normal. Was lively and well-behaved when growing up. Began school at 7 years old, had good academic performance. Completed 10 school years, did her 11th year at evening school because of seizures developed at the age of 7 years. Absence seizures lasting 1-2 minutes and occurring 3-4 times daily every 1-2 days. Received neurologist care at a local health center; does not remember the medications prescribed. Was run over by a car at the age of 16, sustaining no injuries, but soon afterwards developed convulsive seizures with tongue biting and incontinence. Frequency of seizures: 4-5 a month. Most recent treatment: convulex, at 500 mg 3 times daily. Worked a few months as a car wash salesperson. Was dismissed from the job because of the seizures. Resides with the mother. Exhibits changes in personality: irascibility, instability of mood, impaired memory. In 2009, the patient underwent treatment at this inpatient facility; was discharged with a diagnosis of epilepsy with frequent multiform seizures (5 tonic-clonic and 12 absence seizures), mild personality changes′. Most recent treatment: convulex, at 500 mg 3 times daily. In 2012, the patient got married, had pregnancy ending with miscarriage at 9 weeks. Admitted to this facility for an a disability status decision.

Mental status at admission. Neatly dressed, with a trim haircut. Engages in conversation appropriately. Fully oriented. Complains of seizures with loss of consciousness. Guarded, looks in an ingratiating manner at the doctor. Sticks to the point when responding to questions, uses correct phrasal constructions; has difficulty wording some phrases. Thoughts are circumstantial, viscous; tends to linger on her absence seizures, when she abruptly gets motionless for a few seconds while remaining aware of what is going on around. Gives too detailed descriptions of some events when speaking about herself. No signs of psychosis (delirious ideation, perception disorder) identified. Complains of frequently forgetting what she has said, forgetfulness ‘keep losing things’.

Internist's conclusion: no abnormalities identified.

Neurologist's conclusion: encephalopathy (residual, traumatic). Symptomatic epilepsy, personality changes.

Treatment prescribed: convulex, at 500 mg 3 times daily.

AP drug is administered as follows: Day one—20 drops every hour for 6 hours, followed by 20 drops 4 times daily for 28 days.

Day One.

Initiated at 10.00 at 20 drops at hour's intervals

11.00. Irritated, anxious, trying to convince the doctor that her absence seizures can be unnoticeable to others. Gets stuck on this subject, tends to speak in too much detail. Unreceptive to other doctor's questions or forgets them.

12.00. Got acquainted with the fellow patients in her ward but showed no initiative in conversation. Low mood; complains of difficulty finding words, fatigability.

13.00. Complains of ‘no vigor’, ‘feeling sleepy, tired from the journey’.

14.00. Reports having ‘more troublesome thoughts occurring’, explaining that she wishes to undergo a disability status examination. Speech is sluggish, facial expression is calm.

15.00. No complains, looks calm, quiet and reticent. Reports having had ‘no absence seizures so far’

16.00. The patient is asleep.

Day Two.

AP drug is administered at 20 drops 4 times daily (9.00, 13.00, 17.00, 21.00)

Day Seven.

No seizures over the patient's stay at the facility.

Today the patient seems irritable, having low mood; asserts that she has had ‘absence seizure unnoticed by others’ (‘the patient has been closely monitored’) Seems having difficulty in finding words when worried, gets easily exhausted, complains of tiredness. Withdrawn, not eager to interact with hospital staff or fellow patients.

Day Fourteen.

Keeps unnoticed, does not volunteer to see the doctor. Occasionally has low mood, tries to stay in bed for most of the time. Shows no initiative in conversation. The patient herself does not report having experienced any seizures (‘seems like no attacks of motionless’). Thinking remains slow, the tendency to overdetail persists, minor memory impairment.

Day Twenty-One.

Mood is level. Complains of forgetfulness, absent-mindedness, while being able to recall events from the immediate past and giving too detailed descriptions of recent happenings (when her mother came to see her, what they were talking about). Emotionally rigid, unemotional in expressing happiness or dissatisfaction, the time of expressing them is prolonged. Does not report having experienced absence seizures (‘seems like no attacks’). No difficulty sleeping.

Day Twenty-Eight

Calm, mood is normal. No complains of seizures (‘have not had attacks of immobility’) Emotions are better expressed and more positive, displays more interest in conversation, inquires about the discharge date.

No tonic-clonic or similar seizures were recorded over the entire treatment period.

Conclusion. AP drug was administered in combination with other anticonvulsants the received by the patient at the dose employed for over 2 years. At admission to the facility, the patient was irritable, guarded and sulky (low mood). Following treatment with AP drug, she had a better mood and was less guarded and suspicious. There were no ‘attacks of immobility’ as anticipated by the patient, nor were there any tonic-clonic seizures (during her stay at the facility in 2009 she developed 5 tonic-clonic and 12 absence seizures). While torpor and perseveration of thinking persisted, the patient demonstrated more emotions and a better ability to recall sequences of past events as well as displayed more enthusiasm and benevolence in conversation. No sleep problems were recorded.

Thus, the patient's mood, memory, and activity were improved as a result of AP drug used as part of the anticonvulsant treatment administered. No seizures were developed by the patient during the treatment.

### Example 23

A study was conducted to examine the efficacy of the claimed drug, formulated as a solid (tablet) dosage form containing lactose granules impregnated with water-alcohol solution of the liquid activated (potentized), energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a potential difference being applied to electrodes placed in the initial neutral carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug—in treating multiple sclerosis.

Patient K (39 years old) was hospitalized at a neurological unit for ‘Multiple sclerosis. Secondary progressive/remitting phase, relapse’.

Complaints at admission: urination problems, weakness in the lower extremities with occasional inability to get up from the bed.

Based on the medical history, the age of onset is 29 years old; the disease was first experienced as urination disturbances, double vision and staggering walk. A relapse of disease occurred after a year. After a corticosteroid pulse, the neurologic deficit was partially reversed; however, the patient still had complaints of unstable gait, weakness in the legs, and episodes of double vision. The patient was placed on glatiramer acetate and interferon (3, which were discontinued due to severe side effects. He also received neuroactive and vasoactive drug therapies. As a result, the relapsing frequency was once in 4-6 months, with consistent progress of the neurologic deficit; the patient reported experiencing a sufficient memory decline and mood swings; got divorced and left the family. The patient's occupation was programmer; 2 years of grade 2 disability; unemployed.

The patient presented to the hospital with a lower spastic paresis score of 2 in the neurological status, impaired function of the pelvis (urinary retention), low mood, emotional lability an sleep problems. The disability score on the EDSS was 7.

The treatment while in the hospital was metypred at 1000 mg daily for 5 days, with a moderate beneficial effect observed: the score of strength in the lower extremities was increased to 3 with persisting muscle spasticity, the pelvic function impairment was reversed, with no severity reductions in leg ataxia, altered mood status, insomnia with early awaking and poor sleep quality, and memory decline (a score of 25 on the MoCa). A moderate improvement was obtained on the EDSS (score reduction to 6.5).

The patient was prescribed to receive AP drug at 2 tablets 3 times daily for a total of 3 months. Following the treatment, the memory performance on the MoCa test was improved (score of 27) and mood normalized. There were also reductions in spasticity of the lower extremities and coordination defects: the gait was steadier, not shaky.

### Example 24

A study was conducted to examine the efficacy of the claimed drug, formulated as a water solution (liquid dosage form) of the activated (potentized) energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a difference of potentials being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug—in treating obsessive-compulsive disorder.

The tolerability and anxiolytic efficacy of AP drug was examined versus Diazepam in patients with obsessive-compulsive disorder with prevailing anxiety disorders. Inclusion criteria:
1. Subjects aged from 18 to 65 years.
2. Subjects meeting the obsessive-compulsive disorder criteria (ICD-10 code F42).
3. Subjects receiving no treatment with psychoactive agents at least for a week before and at the time of the trial.

The study included 20 patients (8 men and 12 women) aged between 19 and 62 years old. The mean patient age and mean duration of disease were 38.0±1.6 years and 2.1±0.5 years, respectively.

The duration of treatment was 28 months. The patients randomized to AP drug (45) received it at a dose of 20 drops 4 times daily; the rest 35 patients were assigned to receive Diazepam at 15 mg daily.

The patients were assessed for OCD symptom severity at baseline (day 0) and at days 7, 14, and 28.

The rating scales used were

1. the Hamilton Depression Rating Scale (HAM-A);

2. the State-Trait Anxiety Inventory (STAI);

3. a functional state (well-being, energy, mood) inventory (SAN).

Study Results:

At baseline, the total anxiety scores (HAM-A) were 25.88±0.5 in the group of AP drug and 27.97±0.51 in the group of Diazepam. These scores were reduced in both groups after 4 weeks of treatment: to 11.75±0.42 in the group of AP drug and more significantly (to 8.24±0.39) in the Diazepam group (Table 28).

TABLE 28
HAM-A scores

| Study drug | Baseline | | Day 7 | | Day 14 | | Day 28 | |
|---|---|---|---|---|---|---|---|---|
| AP | 25.88 ± | 0.5# | 22.15 ± | 0.67***### | 15.20 ± | 0.55***### | 11.75 ± | 0.42***### |
| (n = 40) | | | | | | | | |
| Diazepam | 26.57 ± | 0.51 | 19.02 ± | 0.51*** | 12.93 ± | 0.64*** | 8.12 ± | 0.39*** |
| (n = 29) | | | | | | | | |

Note:
***p < 0.001 (versus baseline)
# p < 0.05;
### p < 0.001 (versus Diazepam)

HAM-A scores as evaluated at 28 days demonstrated an average reduction of 54.5% in the group of AP drug and that of 70.2% for diazepam-treated patients. Percentages of patients exhibiting treatment response (50% reduction of mean HAM-A score and over) the groups of AP drug and Diazepam were 75% and 100%, respectively. The situational anxiety (STAI) scores were reduced by 30.4% in patients receiving AP drug and by 29.6% in diazepam-treated patients. Similarly, reductions in trait anxiety scores were 26.8% for AP drug and 22.3% for Diazepam.

As shown by the self-rated anxiety scores, the anxiolytic profile of AP drug is beneficial for patients with obsessive-compulsive disorder, with an early onset of effect on anxiety related to a stressful situation, as compared to Diazepam. This is supported by the scoring results on the SAN scale.

Analysis of mean SAN scores indicates similar self-assessment outcomes in patient groups as observed at treatment completion (3.8 for AP drug and 3.6 for Diazepam).

Over the first two weeks, changes in mood self-ratings were more marked in the Diazepam group, which was presumably due to its stronger anxiolytic effect. In contrast, by the end of the treatment, mood improvements as evidenced by the relevant SAN scores were more pronounced for patients receiving AP drug compared to the Diazepam group.

Safety:

All adverse reactions related to the drug treatment were recorded throughout the study. The occurrence of such reactions differed significantly between AP drug and Diazepam: 11.1% (5 patients) and 82.9% (29 patients) of cases, respectively. All cases of adverse reactions in the group of AP drug were not related to the treatment administered. In the Diazepam group, the most commonly developed adverse reactions were sedation, daytime drowsiness, muscle relaxation, and orthostatic disturbances. No adverse effects on vital functions (BP, heart rate, urine and blood tests) were detected for either of the drugs.

### Conclusion

The study showed that AP drug is an effective and safe treatment for neurotic states, in particular obsessive-compulsive disorder accompanied by anxiety disturbances. Its anxiolytic properties are only insignificantly (below the level of statistical significance) inferior to the standard anxiolytic Diazepam. AP′ action was most markedly expressed by anxiety and functioning self-ratings as compared to Diazepam, suggesting the presence of anxiolytic effect not involving ‘behavioral toxicity’ events, which are common to benzodiazepine derivatives. The main advantage of AP drug over Diazepam is the high safety level, as evidenced by almost complete absence of side effects, coupled with a wide titration range. Based on the data gathered, AP drug can be recommended as an anxiolytic treatment for anxiety states in patients with obsessive-compulsive disorders.

### Example 25

A study was conducted to examine the efficacy of the claimed drug, formulated as a solid (tablet) dosage form containing lactose granules impregnated with water-alcohol solution of the liquid activated (potentized), energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a difference of potentials being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug—sequelae of ischemic cerebrovascular accident (CA).

Patient A (woman, 67 years old) sought neurological attention for progressive pain and stiffness as well as substantially limited motion in the right extremities.

More than two years earlier, she had an ischemic cerebrovascular accident, after which she preserved right sided hemiparesis together with increased (spastic) muscle tone. Treatment with vasoactive and nootropic agents had not resulted in noticeable improvement. Muscle weakness of right limbs (worse in the right arm) and muscle spasticity had persisted, impeding the progress of rehabilitation and limiting daily activities (modified Rankin Scale score of 3).

The patient had arterial hypertension; stage achieved—stage I (BP 140/90 mm Hg). Continuous use of enalapril (10 mg/day).

Neurologic status: Mild central paresis of the right facial nerve, right sided hemiparesis scored 2 for the arm and 3.5 for the leg, with spastic muscle tone. Reflex difference D>=S, Babinski signs on the right. Right sided hemihypesthesia. Coordination tests are normal for both left limbs.

AP drug was added to the treatment at a dose of 2 tablet*3 times daily, for 3 months. Following the study treatment, the patient's muscle tone was substantially improved, with a moderate increase in the active range of movement in the hand. Functional capacity was improved (the ultimate mRs score was 2).

### Example 26

A study was conducted to examine the efficacy of the claimed drug, formulated as a solid (tablet) dosage form containing lactose granules impregnated with water-alcohol solution of the liquid activated (potentized), energy-treated carrier, obtained by extreme serial dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a difference of potentials being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug—in treating epilepsy.

Patient V (woman, 37 years old) consulted a local neurologist for persisting attacks of loss of consciousness and convulsion, drowsiness, and impaired memory. For many years, the patient had been receiving follow-up care with an epileptologist for a diagnosis of ‘symptomatic partial epilepsy with secondary generalized tonic-clonic seizures, drug-resistant type’. Seizures: serial.

Duration of disease: since the age of 19 years. Previously the patient was treated with the maximum dose of carbamazepines (1000 mg/day), with a short-term effect, followed by disease relapses. Seizures became more frequent (up to 5 times a month) and tending to occur in series 2-3 seizures a day). The therapy was replaced with 2000 mg/day valproic drugs, with no significant effect but resulting in a weight gain and menstrual cycle disturbances. The patient was prescribed treatment with the maximum dose (300 mg/day) of topiramate, following which the frequency of seizures was reduced (2-3 times a month). The patient began to complain of torpor and impaired memory. Levetiracetam was added to the treatment. At the time of the doctor's visit, she had been receiving 250 mg/day topiramate and 2000 mg/day levetiracetam. The seizure frequency was 2 times a month. The family members reported changes in personality: more circumstantial, more viscous when speaking; had faced workplace issues and had conflicts with colleagues and management; had been unemployed for two years.

AP drug was added to the treatment at a dose of 2 tablet 3 times daily and was administered for 3 months. As a result, the frequency of seizures was somewhat reduced (to 1 seizure a month), the latter being lo longer of serial nature (no more than one seizure during the day). Mood swings were reversed and memory improved.

### Example 27

A study was conducted to examine the efficacy of the claimed drug, formulated as a water solution (liquid dosage form) of the activated (potentized) energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a difference of potentials being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug—in treating pseudoneurotic disorder in patients with organic brain disease.

The efficacy of AP drug was evaluated based on clinical and laboratory data of 30 patients (24 women and 6 men), aged from 42 to 78 years (mean age in the group, 59.9±1.6 years), with cerebral pathology in conjunction with anxiety-depressive disorders.

The comparison group included 20 internal medicine patients (18 women, 2 men; mean age, 75.5±1.8) with various neurological conditions. Organic brain syndrome was represented in the study patients by:
vascular encephalopathy stage 2—53%;
cerebral atherosclerosis—30%;
Posttraumatic encephalopathy—9%;
CA sequelae—13%

Patients in the test group received AP drug at 20 drops 4 times daily for 28 days.

Patients in the group of comparator treatment were administered sedative drugs: phenazepam (8 subjects—as per indications) and sonapax (3 subjects—in cases of severe anxiety).

At baseline, all patients had increased anxiety levels: mild anxiety in 10 patients (33%), moderate anxiety in 14 patients (46%), and severe anxiety in 6 patients (21%). The patients' anxiety levels were reduced on day 14 of drug treatment: 13 patients had mild anxiety (45%), 11 had moderate anxiety (34%) and only 6 patients preserved severe anxiety (21%). On day 28 of AP, anxiety levels were significantly changed in all patients: The number of mild anxiety cases was increased—24 (80%), and there were fewer cases of moderate and mild anxiety (4 (13.3%) and 2 (6.7%), respectively). In the test group, the Hamilton scale score was reduced to 25.7±1.5 (baseline score 35.8±1.2, statistically significant difference—p<0.05).

Following the combination treatment including AP drug, all patients reported improvements in general health, sleep, and mood and normalization of arterial pressure. By the end of the second week of AP drugs, reduction was achieved for the following anxiety symptoms as rated on the Hamilton scale: anxious mood, tension, fearful anticipation, irritability, difficulty in concentration, muscular stiffness, feelings of weakness, insomnia, somatic sensory symptoms, autonomic disturbances (sweating, fewer episodes of palpitation, clearer eyesight, resolution of dry mouth, paresthesia, and tinnitus).

Changes in anxiety symptom ratings were less pronounced in patients receiving phenazepam. In this group, the Hamilton scale score was reduced insignificantly by the treatment - to 31.7±1.5 (baseline score 32.8±1.4, statistically insignificant difference—p>0.05).

The study carried out suggests that AP drug markedly and statistically significantly reduces anxiety levels, improves general health, alleviates irritability and normalizes sleep in patients with various organic brain disorders.

### Example 28

A study was conducted to examine the efficacy of the claimed drug, formulated as a solid (tablet) dosage form containing lactose granules impregnated with water-alcohol solution of the liquid activated (potentized), energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a difference of potentials being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug—in treating Huntington's chorea.

Patient L. had for a year been under follow up with a neurologist for a diagnosis ‘Huntington's chorea’.

The present visit was due to complaints of deterioration of speech (lower voice intensity, loss of fine articulation) and difficulty walking.

Nearly two years earlier, the patient began to notice modifications of voice, and developed uncontrolled movements in his arms and legs, and subsequently impairment of gait. He received treatment with nootropic agent, with no effect observed, and in 2015 he was prescribed to take Akatinol for progressive cognitive impairments.

Positive familial history: Father, uncle and grandmother had Huntington's chorea.

Neurologic status: Alert, oriented to time, place and person. No meningeal symptoms. Sense of smell is intact. Pupils S=D, palpebral fissures OS=OD. Impaired vertical smooth pursuit. Dysarthria. Difficulty in putting the tongue outside the mouth and keeping it up. Moderate extensive chorea-associated hyperkinesia involving the face, extremities and trunk. Muscle strength and range of movement are preserved. Tendon reflexes are brisk; arms S=D, legs D>S, foot clonus D>S. No abnormal reflexes. Muscle tone is normal, no disturbances of sensation. Coordination tests: finger-to-nose test is normal; bilateral dysmetria on the heel-to-shin test. Unsteadiness on the Romberg's test. Slowed walking; the gait is swaying and ‘dancing’. Mnestic decline.

The patient was prescribed a 3-month treatment with AP drug, at 2 tablets 3 times daily. After the treatment, the patient demonstrated less stiffness walking, with walking speed increased; memory was improved.

The drug was shown to produce a beneficial effect on motor functions and cognitive performance.

### Example 29

A study was conducted to examine the efficacy of the claimed drug, formulated as a solid (tablet) dosage form containing lactose granules impregnated with water-alcohol solution of the liquid activated (potentized), energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a difference of potentials being applied to electrodes placed in the initial neutral carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug—in treating sensory disturbances.

For the efficacy evaluation of AP drug in the treatment of sensation disorders, a group of patients was made up of individuals with disorders of sensation due to different disease of the nervous system. The group included 15 patients, 8 of whom had polyneuropathy syndrome, 5 had sequelae of cerebrovascular accident, and 2 were diagnosed with ‘multiple sclerosis’.

In addition to the treatment for the underlying disease, all patients were administered AP drug, at 2 tablets*3 times daily, for a total of 3 months.

At the end of the treatment, changes in sensory symptoms were recorded.

Patients with polyneuropathiy-associated disturbances had a reduced area of hypesthesia.

In patient K, the area of glove-and-stocking surface hypesthesia was reduced to ‘short-grove-and-sock’ hypesthesia.

The severity of sensory symptoms was reduced in patients with sensory disturbances due to CNS diseases (sequelae of cerebrovascular accident, multiple sclerosis).

Patient N, with right-sided hemianalgesia associated with previous ischemic cerebrovascular accident, demonstrated an increase in the feeling of pain up to the hemihypalgesia (ability to feel rubbing with a pointed object).

Patient S., with multiple sclerosis, initially had hemi-disturbances of surface sensation (temperature and pain) on the right side. After the treatment, his temperature sensation was restored and pain sensation in affected limbs improved.

Thus, AP drug was shown to produce a remedial effect on sensory disturbances.

### Example 30

A double-blind, placebo-controlled study was conducted to examine the efficacy of the claimed drug, formulated as a water solution (liquid dosage form) of the activated (potentized) energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a difference of potentials being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug—in treating attention deficit hyperactivity disorder (ADHD) in children.

The study included 20 ADHD patients (15 boys and 5 girls) aged between 6-12 years. Ten patients received treatment with AP drug (Group 1), and the other 10 received placebo (Group 2). The diagnosis of ADHD was based on the ICD-10 criteria for attention-deficit hyperactivity (hyperkinetic) syndrome.

The mean age of participants in the groups of AP drug and placebo was 9.1±0.36 and 8.9±0.32 years, respectively.

For an objective assessment of ADHD symptoms, the Connors Parent rating scale was administered to the patients' parents.

All patients had clinically significant ADHD symptoms, as evidenced by the high baseline scores: 30.8±1.77 in Group 1 and 33.6±1.33 in Group 2.

The drug was administered at a dose of 10 drops 3 times daily, independently of food. The treatment duration was 12 weeks. The dosing regimen, route of administration and length of treatment were the same for AP drug and placebo. The screening examination was performed immediately before treatment initiation, with subsequent assessments at 2, 4, 6, 8, and 12 weeks.

Results. Therapeutic effects of 12-week treatment were more significant in the group of AP drug. Significant improvement displayed as a 50% reduction of the ADHD symptom score compared to baseline was achieved by 3 (30%) patients and moderate improvement was recorded for 2 (20%) patients. At the same time, the majority of (4 (40%)) patients in the placebo group had moderate improvements, and only 1 (10%) patients achieved a significant improvement. The mean total score reduction on the above-mentioned scale was most pronounced at 4 weeks after treatment initiation, and all subsequent differences versus placebo were maintained and became increasingly more significant over the 2nd and 3d months. Thus, improvements in score changes as compared to placebo were achieved by patients treated with AP drug after a month and kept increasing over the two subsequent months of treatment. In the group of AP drug, a significant severity reduction of attention deficits (from 16.4±0.93 to 11.2±0.97; p<0.001, or by 31.7%) and hyperactivity/impulsiveness (from 14.4±1.17 to 8.4±0.93; p<0.001, or by 41.7%) was recorded after 12 weeks of treatment. Thus, there was a more marked beneficial trend observed for AP drug's effects on hyperactivity/impulsiveness symptoms.

One month after treatment initiation, the Connors Parent total score was reduced by 23.4% (from 44.1±2.08 to 33.8±2.11; p<0.001) in the group of AP drug versus only 6.2% in the placebo group (49.7±2.33 to 46.6±2.50; p>0.05). Following 3 months of treatment, a significant score reduction was observed in Group 1 - to 30.5±2.58, or 30.8% (p<0.001) as compared to baseline. The relevant score in the placebo group was 40.5±3.40; i.e., a reduction of 18.5% compared to baseline.

### Conclusion

AP drug has a significant beneficial effect in ADHD (a combination type, moderate) children: the severity of ADHD symptoms is reduced substantially compared to placebo after 12-week treatment at a dose of 10 drops 3 times daily.

### Example 31

A study was conducted to examine the efficacy of the claimed drug, formulated as a water solution (liquid dosage form) of the activated (potentized) energy-treated carrier, obtained by multiple consecutive dilution of the initial matrix solution in an aqueous-alcoholic diluent, combined with external mechanical impact—shaking of each resulting dilution, to a degree of 100²⁰⁰, equivalent to a centesimal (C200) homeopathic dilution (wherein the energy treatment of the initial neutral carrier has been accomplished by exposing it to an electric current caused by a difference of potentials being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug—in treating mental and behavioural disorders due to use of alcohol, moderate stage.

Patient J. (woman, 49 years old).

Diagnosis: ‘Mental and behavioral disorders due to use of alcohol, moderate stage. Delirium, acute intoxication with delirium. Dependence syndrome, withdrawal state with delirium’.

Case history data: positive family history—alcoholic father. Completed secondary education (10 years of comprehensive school). Remarried, two first-marriage children (biological father—tragically deceased). Unemployed. Lived on survivors' benefits, current occupation—looking after a farm household. A year earlier—developed binge drinking, with binges lasting up to a week. Periods clear of alcohol abuse: from 2-3 days to a month. Uses distilled drinks, tolerance: 500 ml a day. Enjoys being inebriated, feeling ‘joyful and careless’. Underwent outpatient treatment with a substance abuse professional half a year earlier, without coding therapy. The latest bout of drinking lasted a month, followed by 4 days of abstinence. The discontinuation of alcohol consumption induced a psychotic state, which was the reason for bringing the patient to this inpatient unit.

Mental state during the visit: anxious, confused; the patient was crying and appeared disoriented to place and time. When in the unit, the patient slept uninterruptedly at night, and was disoriented.

During stay in facility: Unkempt hair, untidy clothes. Looking sleepy. Clumsiness in movement. Marked general tremor, especially in the hands; increased sweating. Puffy face. Absent-minded, inattentive; answers questions with one word responses. Roughly oriented as to place, can not recall current date accurately (‘even have no idea of today's date’), recalls her named correctly. Perplexed about the necessity of hospitalization, stating that she ‘has been off drinking’. Says uncritically that she ‘was chased by tractors’ because she ‘could see light ahead’. Low mood.

Treatment prescribed: intravenous saline drips, riboxine, asparcam, magnesium sulfate solution, B vitamins, maxidole.

AP Drug Regimen:

Day one: 20 drop every 20 minutes for 6 hours, followed by 20 drops 4 times daily the next day and onwards, for a total of 5 days.

Day One.

10.20. The patient is seated on her bed, looking around absently. Answers questions apathetically, says she wants to sleep.
10.40. Asleep, not fully relaxed, moving her fingers, frowning.
11.00. Awoken to go to the toilet; the gait is unsteady, lethargic. The facial expression is calm; on interview, responses are limited to the questions asked, not extended.
11.20. Fast asleep, in an awkward position, without hearing calls.
11.40. Has been given by a staff member a dose of AP drug; looks sleepy, relaxed, and flabby. Fell immediately asleep.

### 12.00. Asleep.

12.20. Awoken, lying peacefully in the bed; reports feeling better, more ‘tranquil and uplifted’ Less tremor. Movements are somewhat slowed. No active mental symptoms.
12.40. Staying in bed, with episodic dosing. Reacts calmly to the environment.
13.00. Asked to come to the doctor's office. The hair is done; has come smiling in a friendly manner. Calm facial expression, the face is slightly reddened. Answers are relevant. Noted having been drinking excessively (‘slipped back into drinking—a common thing to me’) Telling willingly about her past-life memories (‘there were tractors following me and lighting the way in front of me; it was fearful’. Uncritical, perplexed (‘wonder what they were up to’). Somewhat fidgety, absent-minded; hand tremor is noticeable.
13.20. Has gone back to bed, explaining she wants to ‘sleep off’; fell asleep easily.
13.40. The patient is asleep.
14.00. Awoken for lunch, looks satisfied with her state (‘feeling peaceful, have no problems; can not recall what has been happening to me’ Perplexed whether ‘it is because of vodka’
14.20. Seated on the bed, talking with the fellow patient. The patient is in bed again, complaining of feeling tired.
14.40. Awoken to take AP drops; then falls asleep again.
15.00. Fast asleep, breathing smoothly. Skin colour is normal.
The facial expression is calm and relaxed.
15.20. The patient is asleep.

### 15.40. Asleep.

16.00. Awake, walking around the unit; looks calm. Conversant, oriented to place; after concentrating, she recalls the date correctly based on memories of some life events. Reports a remaining feeling of ‘internal shaking’. Hand tremor is reduced.
Got to sleep the previous day at 21.30; slept through 05.40 without waking. Explained to the staff that she was used to getting up early. No psychoactive therapy.

Day Two.

AP drug—at 20 drops 4 times during the day (09.30, 13.30, 17.30, 20.30).
10.00 Called into the doctor's office. Dressed neatly. Fully oriented. No psychotic signs (delirious ideation, perception problems) identified. Complaining of a lack of vigor, fatigability. Hand tremor (not severe) is noticeable. Mood is unstable; the patient is easily disposed to tears when recalling her children, has a feeling of guilt for them. Lack of criticism regarding the psychosis experienced.

Day Three.

AP drug—at 20 drops 4 times during the day (09.30, 13.30, 17.30, 20.30).
12.00. Looks calm and friendly. No tremor identified. Facial colour is normal; the face is no longer puffy, with clearly distinguishable and vivid emotional expression. No psychotic signs. Seems perplexed when telling about her unhealthy experiences (‘how come I could catch such an apparition?’). ‘I know vodka can cause this, but I had never thought it could happen to be’. Willing to cut down on drinking (‘I have to run the household—there is no much time for drinking’). Sleeps well.

Day Five.

AP drug—at 20 drops 4 times during the day (09.30, 13.30, 17.30, 20.30).

12.00. Looks calm and friendly. Has no complains; reports having begun to help the staff. ‘I'm feeling internally tranquil, as if relieved of something’. Realizes that the cause of psychosis experienced was alcohol. Promises to give up drinking, ‘as well as social drinking’. Sleeps well at night, gets to sleep during nap time.

### Conclusion

The patient presented in a psychotic state, manifested as insomnia, hallucinatory experiences, altered level of conscientiousness, and autonomic symptoms. AP drug was initiated on the first day of inpatient care, and was co-administered with detoxification therapy. Sedative effect was achieved; the patient slept a lot during the first day; a gradual reduction of alcohol withdrawal events was observed. The patient could sleep at night without additional neuroleptics having been administered. The patient had moderate asthenia, and short-term mood swings. No complaints were reported by the patient on the fifth day of treatment; she had a stabilized mood, and was reasonably critical about the psychosis experienced.

AP drug co-administered with detoxification agents produced a marked sedative effect and exhibited an antipsychotic action resulting in elimination of alcohol withdrawal events and psychopathologic symptoms within the first 24 hours.

### Example 32

A study was conducted to examine the efficacy of the claimed drug, formulated as a solid (tablet) dosage form containing lactose granules impregnated with water-alcohol solution of the liquid activated (potentized), energy-treated carrier, obtained by multiple consecutive dilution of the initial matrix solution in an aqueous-alcoholic diluent, combined with external mechanical impact—shaking of each resulting dilution, to a degree of 100200, equivalent to a centesimal (C200) homeopathic dilution (wherein the energy treatment of the initial neutral carrier has been accomplished by exposing it to an electric current caused by a difference of potentials being applied to electrodes placed in the initial neutral carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug—in treating consequences of stroke.

Purpose of Study:

To evaluate effectiveness and safety of AP drug in treatment of consequences of stroke

Materials and Methods.

Study included patients who suffered stroke, ishemic type of differing age. Patients suffered decrease in ability to perform daily functions (3-4 in accordance with modifyied scale of Rankine (MRs).

Patients received monotherapy of AP drug, 2 tablets, 3 times a day for 28 days. For some patients, the study continued for 6 months.
The study evaluated the dinamics of patients functionality on mRs scale 4 weeks after the initiation of treatment and 6 month after initiation of treatment in comparison with period before initiation of treatment.

The results are presented in the following table.

| | mRs | mRs | |
|---|---|---|---|
| Patients | before | after | |
| # | therapy | therapy | Subjective evaluation |
| 001 | 3 | 2 | Significant improvement of self control: able |
| | | | to work in the garden |
| 002 | 4 | 4 | Improvement in general condition, reduction |
| | | | in headaches, more initiative |
| 003* | 3 | 3 | No changes |
| 004* | 3 | 3 | Improvement in general condition, better |
| | | | able to handle stress, better mood. |
| 005 | 1 | 1 | Reduction in feeling disturbances (the area |
| | | | of hypestasia reduced in size), less than 2 |
| | | | months from initiation of treatment, the |
| | | | patient returned to work (driver). |
| 006 | 3 | 3 | Improvement in emotional conditions, |
| | | | improvement of control, improvement in |
| | | | cognitive function (solves crossword puzzles, |
| | | | improvement in discorrdination of movement |
| 007* | 4 | 4 | Positive dynamics as improvement in power |
| | | | of limbs. Did not participate in prolongation |
| | | | of study. |
| 008 | 3 | | The patient was able to return to work |
| | | | (computer expert), clear improvement of |
| | | | memory, improvement in mood, increase in |
| | | | the power of limbs |
| 009 | 4 | 4 | No changes |
| 010 | 3 | 2 | Significant improvement in memory |

Results.

The study included 10 people (6 men, 4 women) 59 to 72 years of age, medium age was 66.1±4.01 years. The time from stroke was changing from 0.9 to 9 years (average 2.48 years). Therapy with AP drug improved emotional atmosphere (anxiolytic, emotion-stabilizing activity), which manifested itself in improvement of general conditions, cessation of headaches, improvement of mood. 80% patients manifested improvement of cognitive functions./ These effects are likely connected with nootropic activity of the drug in study.

The improvement manifested with the drug in study is comparable with modern rehabilitation methodologies.

### Conclusions

AP drug has nootropic effect in treatment of consequences of stroke.

### Example 33

An open, non-comparative study was conducted to examine the efficacy and safety of the claimed drug, formulated as a water-alcohol solution (liquid dosage form) of the activated (potentized) energy-treated carrier, obtained by multiple consecutive dilution of the initial matrix solution in an aqueous-alcoholic diluent, combined with external mechanical impact—shaking of each resulting dilution, to a degree of 100200, equivalent to a centesimal (C200) homeopathic dilution (wherein the energy treatment of the initial liquid carrier has been accomplished by exposing it to an electric current caused by a difference of potentials being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug, where it was used for treating cerebral palsy.

Material and Methods.

The study included children 3 to 14 years of age with motor and pseudobulbar effects of cerebral palsy.

The patients received AP drug 10 drops/6 times a day for 1 week, then 10 drops, 3 times a day every day. The length of the study was 8 weeks.

At 4 and 8 weeks, the investigators evaluated dynamics of motor function based on Gross Motor Function Measure—GMFM-66.

During the first month, the patients received the study drug together with broad spectra of rehabilitation measures, including physical therapy, speech therapy, and massage. The second month of the study, the patients spent at home on monotherapy of AP drug.

Statistical analysis was carried out as single factor covariant analysis. The valye of the sum scope before the beginning of therapy served as the covariant.

Results:

The study included 20 people (16 boys, 4 girls) suffering from various forms of cerebral palsy: dyplegia, hyperkinetic form, atonic-astatic form, hemiparetic form. Average age of study participants was. 3.18±0.61 years.

The results are presented in the table:

| | | GMFM | GMFM | |
|---|---|---|---|---|
| | | after 1 | after 2 | |
| | GMFM | month | months | |
| Patient | before | of | of | Subjective evaluation of patient's |
| # | therapy | therapy | therapy | condition |
| 001 | 25 | 29 | 32 | Spastic dyplegia. Improvement |
| | | | | of muscle tone, sits better, |
| | | | | become calmer |
| 002 | 117 | 141 | 152 | Spastic dyplegia. Improvement of |
| | | | | muscle tone improvement in |
| | | | | movement and joint functioning, |
| | | | | reduction in ataxia, improve, ment |
| | | | | in fine motor skills, improvement in |
| | | | | understanding of speech, |
| | | | | improvement in vocabulary., |
| 003 | 133 | 137 | 138 | Atonic-astatic form. Epilepsy. |
| | | | | Improvement in physical condition, |
| | | | | reduction in pseudobulbar |
| | | | | syndrome, improvement in |
| | | | | adaptation, less irritability. |
| 004 | 130 | 138 | 146 | Hemiparetic form. Improved muscle |
| | | | | tone, improved the breadth of |
| | | | | movement, improved concentration |
| | | | | of attention, increased vocabulary. |
| 005 | 50 | 60 | 60 | Spastic dyplegia. Improved muscle |
| | | | | tone, decreased stiffness of joints, |
| | | | | decreased ataxia. Walks more |
| | | | | certainly and for greater distances. |
| 006 | 125 | 131 | 132 | Spastic dyplegia. Well adapted to |
| | | | | the environment, improved muscle |
| | | | | toner, insignificant reduction in |
| | | | | dysforia. |
| 007 | 146 | 152 | 165 | Spastic dyplegia. Improved muscle |
| | | | | tone, increased breadth of |
| | | | | movement in certain joints, |
| | | | | improved understanding of speech, |
| | | | | increased vocabulary. |
| 008 | 84 | 117 | 123 | Spastic dyplegia. No changes. |
| 009 | 88 | 95 | 101 | Spastic diplegia. Decreased |
| | | | | hypersalivation, anxiolytic effect. |
| 010 | 60 | 72 | 86 | Spastic dyplegia Improvement in |
| | | | | orthopedic tests, improvement in |
| | | | | understanding of speech. |
| 011 | 128 | 134 | 141 | Spastic dyplegia |
| 012 | 42 | 56 | 61 | Spastic dyplegia. No changes. |
| 013 | 42 | 56 | 61 | Spastic dyplegia. Normalized |
| | | | | behavior. |
| 014 | 125 | 131 | 136 | Left side hemiparesis. No changes. |
| 015 | 158 | 164 | 166 | Left side hemiparesis. Decreased |
| | | | | ataxia. |
| 016 | 131 | 142 | 144 | Spastic dyplegia. No changes |
| 017 | 77 | 87 | 87 | Spastic dyplegia. No changes. |
| 018 | 17 | 20 | 25 | Spastic dyplegia. No changes. |
| 019 | 95 | 101 | 108 | Spastic dyplegia. No changes |
| 020 | 35 | 52 | 57 | Atonic-astatic form. Epilepsy. No |
| | | | | changes. |

Overall, there was observed improvement of muscle tone with increase in breadth of movement in joints, 2 patients (10%) exhibited decrease in pseudobulbar effects. The majority of pateitns exhibited improvement in adaptation to the environment, better understanding of speech.

The average improvement of motor function in GMFM-66 scale was 11.2±8.3 points on the 2^{nd} visit, and—16.4±9.6 points on the 3^{rd} visit. Therefore, the positive effect of the AP drug was observed after leaving the hospital and there was an im[rovement and growth of the positive effect f inpatient treatment.

Additions, there was carried out an analysis of dynamics of changes separately on different domains; A—the position of the patient on the coach, B—the position of the patient standing on the knees, D—standing; D—walking. Results are illustrated in the table below and in FIG. 9.

Dynamics of GMFM-66

| | Days | | | | | |
|---|---|---|---|---|---|---|
| | Day 0 | | Week 4 | | Week 12 | |
| | | Relative | | Relative | | Relative |
| | Total | dy- | Total | dy- | Total | dy- |
| Variable | GMFM | namics | GMFM | namics* | GMFM | namics* |
| Average | 95.3 | 0 | 106.5 | 11.2 | 111.7 | 16.4 |
| Deviation | 45.1 | 0 | 46.1 | 8.3 | 45.7 | 9.6 |
| Min | 17 | 0 | 20 | 3 | 25 | 5 |
| Max | 158 | 0 | 177 | 31 | 178 | 39 |

*On domain A on 1^{st}, 2^{nd} and 3^{rd} visit reliable changes of total GMFM-66 was not observed

### Conclusions

Therapy with AP drug has positive influence on patients with cerebral palsy, improved muscle tone, increases range of motions in joints, reduces pseudobulbar affect. The patients improved adaptation to the environment and understanding of speech.

### Example 34

An open, non-comparative study was conducted to examine the efficacy and safety of the claimed drug, formulated as a water-alcohol solution (liquid dosage form) of the activated (potentized) energy-treated carrier, obtained by multiple consecutive dilution of the initial matrix solution in an aqueous-alcoholic diluent, combined with external mechanical impact—shaking of each resulting dilution, to a degree of 100200, equivalent to a centesimal (C200) homeopathic dilution (wherein the energy treatment of the initial liquid carrier has been accomplished by exposing it to an electric current caused by a difference of potentials being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug, where it was used for treating autism.

Purpose:

evaluate effectiveness and safety of AP drug in treating autism.

Materials and Methods.

Study included children 3 to 14 years of age with autism of behavior. Before the drug in the study was administered the autistic symptoms were evaluated using quantitative scale of children autism—CARS (Childhood Autism Rating Scale).

The evaluation of general clinical impression was carried out using—Clinical Global Impression (CGI).

The length of therapy was 8 weeks. The drug was administered 10 drops 6 times a day 15 minutes before food intake the first week of treatment and 10 drops 3 times a day 15 minutes before food intake for the rest of the study.

On 3^{rd} day of study, there was carried out clinical evaluation of somatic and neurological status of the patient.

4 weeks and 8 weeks after the beginning of the study, there was carried out quantitative evaluation of the depth of autistic symptoms using CARS and CGI scales.

Results.

The results are presented in the Table

| Patient | CARS Dynamic¹ | Subjective evaluation\ |
|---|---|---|
| # | Initial - 4 weeks - 8 weeks | Initial - 4 weeks - 8 weeks |
| 001 | 38-35-29.5 | Positive dynamics |
| 002 | 38.5-34-32 | Positive dynamics |
| 003 | 40.5-34.5-30.5 | Positive dynamics |
| 004 | 41.5-38.5-32.5 | Positive dynamics |
| 005 | 40-36.5- 34 | Positive dynamics |
| 006 | 44-37.5-31 | Positive dynamics |
| 007 | 41-39-34 | Positive dynamics |
| 008 | 44-44-40.5 | Positive dynamics |
| 009 | 40-37.5-33 | Positive dynamics |
| 010 | 39-35-28 | Positive dynamics |

For statistical analysis of data, ANOVA was used with subsequent post-hoc analysis (Tukey HSD test) for fractions. In the absence of control group, binominal test for evaluation of frequency was used.

The children in the study exhibited significant reduction in autism symptoms at the 4 weeks mark (7.1 points at 4 weeks mark and 6.9 points at week 8). Total dynamics in the study was 14 pointsStatistical analysis of data showed that the difference in the CARS scale on visits 1 and 2, 1 and 3, and 2 and 3 differ in statistically significant manner. (p=0.028, p=0.0003, p=0.033, respectively).

Table Dynamics on CARS scale

| Visait | Dynamics on CARS scale | p-value |
|---|---|---|
| 1 | 0 | |
| 2 | −7.1 | 0.028 |
| 2-3 | −6.9 | 0.033 |
| 1-3 | −14 | 0.0003 |

¹The severity of autistic disorders: 15-29 - lack of autism;
30-36 - mild to moderate autism;
37-60 - Severe (severe) autism

Additionally, there was analyzed fraction of patients with improvement of communicative function (point CARS 11), with improvement of socialization (CARS points 1, 2, 3, 6, 13), with reduction in stereotypes (CARS 4 and 5). It was demonstrated that improvement in the main results from change in measurement of socialization (comparison with two sides binominal analysis against probability 0.5-fraction of patients with improvement of socialization is far greater than 0.5 at 2^{nd} visit (p=0.0214), as well as the 3^{rd} visit (p=0.002)).

Analysis of fraction of patients with improvement of communicative function and with reduction of stereotypes did not show meaningful difference from 0.5 on the 2^{nd} and the 3^{rd} visit.

General clinical evaluation of patients was carried out on 4^{th} week and 8^{th} week of the study. Average score on the CGI scale, which was initially 3.1, decreased to 2.9 on the 4th week of the study and to 2.2 at the end of the study.

Dynamics on the CGI*Scale

| Visit | Dynamics on the CGI scale | p-value |
|---|---|---|
| 1 | 0 | HΠ |
| 2 | −0.2 | 0.58 |
| 2-3 | −0.9 | 0.0003 |
| 1-3 | −0.7 | 0.0045 |

Based on doctor's evaluation, 90% of the patients were transferred from“lightly ill” category to “border” category. One patient was evaluated as “not ill.

### Conclusions

AP drug showed its effectiveness in correction of illnesses of autism spectra. A statistically significant improvement in socialization function was demonstrated.

### Example 35

A comparative study was conducted to examine the efficacy and safety of the claimed drug, formulated as a solid dosage form containing lactose granules impregnated with water-alcohol solution of the liquid activated (potentized) energy-treated carrier, obtained by multiple consecutive dilution of the initial matrix solution in an aqueous-alcoholic diluent, combined with external mechanical impact—shaking of each resulting dilution, to a degree of 100200, equivalent to a centesimal (C200) homeopathic dilution (wherein the energy treatment of the initial neutral carrier has been accomplished by exposing it to an electric current caused by a difference of potentials being applied to electrodes placed in the initial neutral carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug, where it was used for treating epilepsy

Purpose:

evaluate effectiveness and safety of AP drug in treatment of pharmacoresistant epilepsy.

Materials and Methods.

The study included outpatient subjects both genders 18 to 70 years of age with confirmed diagnoses of epilepsy with existing antiepilepsy therapy with epileptic episodes of frequency of not less than 3 a month.

Results

At present time, the study has results of treatment of 11 pateints.

Preliminary analysis showed that the AP drug does not change the extent or frequency of epileptic episodes.

However, subjectively, the patients noted improvement in emotional tone, quality of sleep.

Table Preliminary results of the study

| | N Epileptic | | | | |
|---|---|---|---|---|---|
| | episodes | | | | |
| | at the | | QOLIE | | |
| Patient | beginning | | I visit/ | | |
| # | of study | HADS | II visit | NHS3 | MoCa |
| 221401 | 3-3-3/ | 21-22 | 84 + 50/ | 18/2- | 23-25 |
| | 2-2 | | 75 + 50 | 15/0 | |
| 221402 | 7-6-6/ | 21-21 | 97 + 0/ | 1/11/22- | 25-28 |
| | 8-6 | | 98 + 0 | 1/6/- | |
| 221403 | 4-5-5/ | 14-11 | 121 + 70/ | 12/16- | 27-26 |
| | 5-4 | | 110 + 70 | 6/0 | |
| 221404 | 3-3-3/ | 14 | 98 + 50 | 18 | 22 |
| 220606 | 14-13-12/ | 22/27 | 113 + 50/ | 22/22 | 16/12 |
| | 9-10 | | 93 + 40 | | |
| 220607 | 04-03-05/ | 19/16 | 93 + 50/ | 4/11- | 16-18 |
| | 1-5(2) | | 109 + 60 | 2/11 | |
| 220603 | 19-16-20/ | 17-21 | 103 + 50/ | 10-3 | 23-26 |
| | 18-16 | | 99 + 30 | | |
| 220604 | 3-3-3/ | 19-23 | 94 + 40/ | 9-8 | 20-25 |
| | 4-3 | | 102 + 30 | | |
| 220605 | 5-4-5/ | 18-17 | 87 + 40/ | 6/9- | 26-24 |
| | 3-4 | | 88 + 50 | 8/11 | |
| 220601 | 14-13-12/ | 22/27/27 | 113 + 50/ | 22/22/22 | 16/12/17 |
| | 9-10/ | | 93 + 40/ | | |
| | 7-11-9 | | 94 + 40 | | |
| 220602 | 04-03-05/ | 19/16 | 93 + 50/ | 4/11- | 16-18- |

### Conclusions

there was no direct evidence of effect of the drug in study on epileptic activity. There was a marked influence of the drug on phycoemotional state of the patients. The study will continue.

### Example 36

An open, non-comparative study was conducted to examine the efficacy and safety of the claimed drug, formulated as a solid (tablet) dosage form containing lactose granules impregnated with water-alcohol solution of the liquid activated (potentized), energy-treated carrier, obtained by multiple consecutive dilution of the initial matrix solution in an aqueous-alcoholic diluent, combined with external mechanical impact—shaking of each resulting dilution, to a degree of 100200, equivalent to a centesimal (C200) homeopathic dilution (wherein the energy treatment of the initial liquid carrier has been accomplished by exposing it to an electric current caused by the difference of potentials being applied to electrodes placed in the initial liquid carrier and which is proportional in amplitude to an amplified combined, summed signal, formed by summing up the resultant signals—differences of biopotentials of individual referential derivations recorded in five healthy donors using the internationally recognized 10-20 system, which involves 19 active recording electrodes placed in regular positions all over the scalp and one reference electrode located on the ear-lobe)—further referred to as AP drug, where it was used for treating dementia.

Purpose:

to evaluate safety and efficacy of the AP drug in treating patients with dementia

Material and Methods.

The study included patients of both genders of 60 to 85 years of age suffering from dementia of different etiology. A study doctor was conducting an evaluation of cognitive functions (Montreal cognitive Assessment—MoCA) and the evaluation of positive, negative and effective symptomatics using (The Brief Psychiatric Rating Scale (BPRS). The caretakers was asked to fill out a questionnaire to evaluate behavioral and phycopathological symptoms of dementia (Neuropsychiatric Inventory—NPI). Existing therapy was registered at the outset. The AP drug was administered during 3 months during which the patients made 2 more visits to the doctor. During second visit, the doctor carried out control of co-existing therapy, evaluated safety of the study drug, evaluated patient compliance with the therapy, counted retirned pills, gave out pills until the next visit. In the course of 3^{rd} visit, the doctor carried out collection of patients complains, registered the data of objective patient evaluation, carried out control of pre-existing therapy and study drug, counted retirned pills, gave out pills until the next visit. The doctor filled out MoCA scale, the caretaker filled out another NPI questionnaire.

To evaluate statistical significance, ANOVA was used with subsequent post-hoc analysis (Tukey HSD test) for fractions. Due to lack of control group, binominal analysis was used to evaluate frequency of events.

Results.

The study included 20 patients (16 men and 4 women). Average age 77.85±8.54 years.

It was determined that the cognitive functions did not change significantly during the study. (11.35±1.21 at the beginning of therapy and 11.90±1.21 after 3 months);

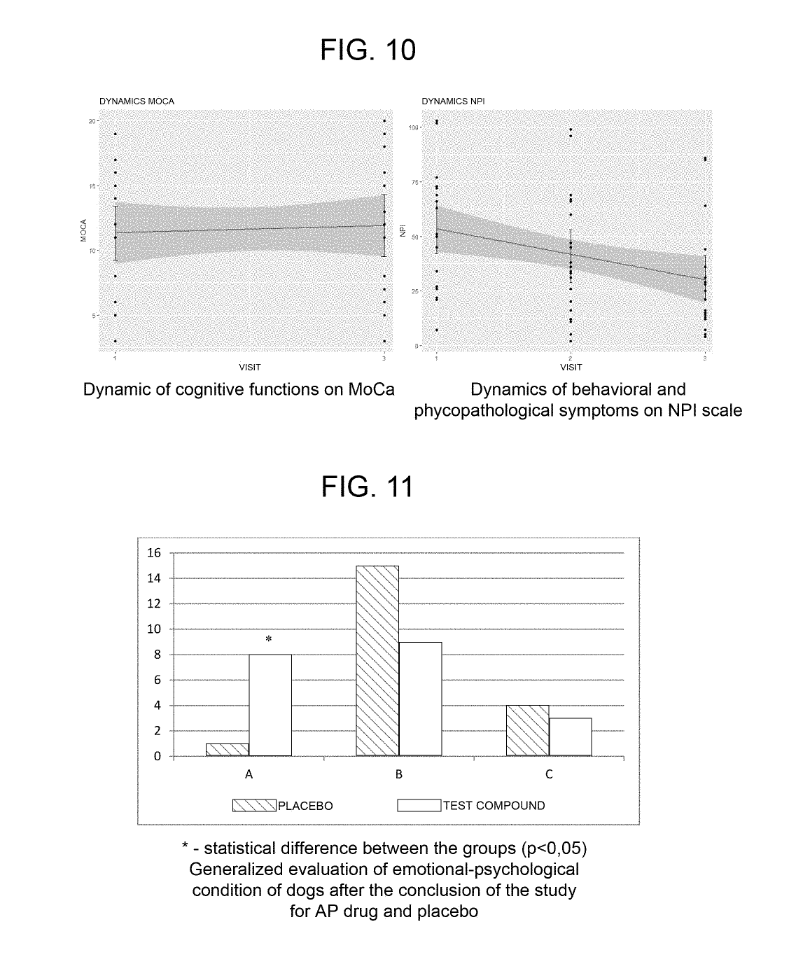

The behavioral and pathological symptom of dementia were reduced during the study from 54.05±6.06 30.75±6.06 (p=0.024). Subsequent post-hoc analysis showed gradual reduction in phycopathological symptoms 23.3 points during the study, with statistically significant differences at 1 month after initiation of the study (p=0.023). Results are illustrated in the tables below and in FIG. 10.

Table Evaluation of cognitive function on MoCa scale

| Visit | Mean MoCa, (mean ± SE) | p-value* |
|---|---|---|
| 1 | 11.35 ± 1.21 | p = 0.749 |
| 3 | 11.90 ± 1.21 | |

*regression analysis (ANOVA)

Table Evaluation of behavioral and psychopathological symptoms of
dementia on NPI scale

| Visit | Mean NPI, (mean ± SE) | p-value* |
|---|---|---|
| 1 | 54.05 ± 6.06 | p = 0.024 |
| 2 | 40.65 ± 6.06 | |
| 3 | 30.75 ± 6.06 | |

*regression analysis (ANOVA)

Dynamics of behavioral and phycopathological symptoms of dementia
on NPI scale

| Visit | Change of NPI score | p-value* |
|---|---|---|
| 2-1 | −13.4 | p = 0.269 |
| 3-1 | −23.3 | p = 0.023 |
| 3-2 | −9.9 | p = 0.484 |

### Conclusions

The AP drug normalizes behavior and mood of dementia patients, reducing the expression of phycopathological symptoms, does not have substantial effect on cognitive functions

### Example 37

A placebo-controlled study was conducted in dogs to examine the efficacy and safety of the claimed drug, formulated as a water-alcohol solution (liquid dosage form) of the activated (potentized) energy-treated carrier, obtained by multiple consecutive dilution of the initial matrix solution in an aqueous-alcoholic diluent, combined with external mechanical impact—shaking of each resulting dilution, to a degree of 100200, equivalent to a centesimal (C200) homeopathic dilution (wherein the energy treatment of the initial liquid carrier has been accomplished by exposing it to an electric current caused by a difference of potentials being applied to electrodes placed in the initial liquid carrier and which is proportional in amplitude to an amplified combined, summed signal, formed by summing the resultant signals—biopotential differences of individual referential derivations recorded in five healthy donors using the internationally recognized 10-20 system, which involves 19 active recording electrodes placed in regular positions all over the scalp and one reference electrode located on the ear-lobe)—further referred to as AP drug, where it was used for treating dogs to improve their well-being and to reduce stress exerted on dogs.

Invetigation was conducted on 44 dogs. Animals were separated into 4 groups. In two groups animals received AP drug, in two groups placebo. Every day during 14 days, the results of the clinical observation as werll as emotional-phycological state of the dogs were recorded. To exclude cancellation syndrome, the dogs were observed for 3 days after the drug administration was completed. At the end of the study, an overall evaluation of the dogs' condition was carried out. It was that there were no statistically significant difference in clinical parameters between dogs in the study groups and in the placebo groups. However dogs receiving AP drug exhibited statistically significant improvement in emotional state on the RASS scale in comparison with the dogs receiving placebo.

Dose, Scheme and Method of Administration

Test composition were administered to 44 dogs using a syringe orally once a days for 14 days. Daily dose of each sample was 0.2 ml per 1 kg of body weight but not more than 10 ml per one animal.

Results of the Study

On the graph in FIG. 11, there are shown cumulative grades A, B and C which characterize emotional-psychological status of dogs after administration of the AP drug and placebo.

Generalized Evaluation of Emotional-Psychological Condition of Dogs after the Conclusion of the Study for AP Drug and Placebo

On the basis of the Fisher criteria, statistically significant differences between the groups were revealed. In groups of AP drug administration the fraction of dogs with marked improvement in emotional-psychological condition was statistically significantly higher.

What is claimed is:

1. A pharmaceutical composition comprising a neutral carrier treated with energy proportional to bioelectric potentials of the human brain or nervous tissue.

2. The pharmaceutical composition of claim 1, wherein said energy treatment is carried out by passing electric current through an initial liquid carrier, which is later impregnated onto said solid neutral carrier.

3. The pharmaceutical composition of claim 1, wherein said energy treatment is carried out by passing electric current through an initial liquid carrier, which is the neutral carrier administered as a pharmaceutical composition.

4. The pharmaceutical composition of claim 2, wherein said energy-treated liquid carrier is subjected to multiple consecutive dilutions based on homeopathic technology prior to said impregnation.

5. The pharmaceutical composition of claim 2 or claim 3, wherein said initial liquid carrier is a distilled water or water-alcohol mixture with conductivity not more than 0.1 mS/m at 25° C.

6. The pharmaceutical composition of claim 2, wherein said electric current results from a difference of electric potentials applied to at least two electrodes placed into said liquid carrier, the amplitude of said difference of electric potentials being proportional to bioelectric potentials read off different locations of human scalp.

7. The pharmaceutical composition of claim 6, wherein the difference of electric potentials applied to the at least two electrodes placed into said liquid carrier is proportional in the amplitude to the difference of bioelectric potentials registered by active electrodes placed on different points of human scalp.

8. The pharmaceutical composition of claim 7, wherein the difference of potentials between the at least two electrodes placed into said liquid carrier is proportional in the amplitude to difference of bioelectric potentials between an active electrode reading bioelectric potential at a location of the human scalp and a reference electrode placed on the ear-lob.

9. The pharmaceutical composition of claim 2, wherein the difference of potentials being applied to the pair of electrodes in the initial liquid carrier is proportional in amplitude to the difference of biopotentials recorded in a bipolar derivation consisting of active recording electrodes placed at different locations on the surface of the human scalp.

10. The pharmaceutical composition of claim 2, wherein the difference of electric potentials being applied to the at least two electrodes in the initial carrier is proportional in amplitude to an amplified summed or averaged signal formed by adding up the differences in bioelectric potentials from a series of referential derivations of recording electrodes that contribute to different channels from different locations on human scalp.

11. The pharmaceutical composition of claim 2 or claim 3, wherein the difference of electric potentials being applied to the at least two electrodes in the initial liquid carrier is proportional in amplitude to an amplified summed or averaged signal formed by adding up the bioelectric differences of potentials from referential derivations of active electrodes contributing to different channels from different locations on the scalp of a donor—the individual the biopotential recordings from whom are used in preparing the pharmaceutical composition.

12. A method of treating diseases or conditions of central nervous system comprising administering to a patient in need thereof the pharmaceutical composition of claim 2, claim 3, claim 4, claim 5, claim 6, claim 7, claim 8, claim 9, claim 10 or claim 11.

13. The method of claim 12, further comprising administering a second pharmaceutical composition, the first pharmaceutical composition being prepared by exposing said liquid carrier to the energy treatment using the difference of potentials applied to the at least two electrodes in the initial carrier, which difference of potentials being proportional in amplitude to the difference of biopotentials of electrodes recording data from different sites of the scalp of the same patient that will be subject of administration, and the second pharmaceutical composition being obtained by exposing the liquid carrier to the energy treatment using the difference of potentials which is proportional in amplitude to the difference of biopotential of electrodes recording from different sites of the scalp of a donor.

14. The method of claim 12, further comprising administering a second pharmaceutical composition, both pharmaceutical compositions prepared using biopotentials of different donors.

15. A method of treating alcoholism in a human patient comprising administering an effective amount of the pharmaceutical composition of claim 2, claim 3, claim 4, claim 5, claim 6, claim 7, claim 8, claim 9, claim 10 or claim 11 to the patient in need thereof.

16. The method of claim 15, which comprises administering pharmaceutical composition of claim 4.

17. The method of claim 16, wherein said pharmaceutical composition is C200 dilution.

18. The method of claim 16, wherein said pharmaceutical composition is prepared by using brain biopotentials of five healthy donors.

19. The method of claim 16, which is in solid form.

20. A method of treating psychosis in a human patient comprising administering an effective amount composition of claim 2, claim 3, claim 4, claim 5, claim 6, claim 7, claim 8, claim 9, claim 10 or claim 11 to the patient in need thereof.

21. The method of claim 20, which comprises administering pharmaceutical composition of claim 4.

22. The method of claim 21, wherein said pharmaceutical composition is C200 dilution.

23. The method of claim 16, wherein said pharmaceutical composition is prepared by using brain biopotentials of five healthy donors.

24. The method of claim 16, which is in solid form.

25. The method of claim 20, wherein said psychosis is the result of schizophrenia.

26. A method of treating posttraumatic stress disorder in a human patient comprising administering an effective amount of pharmaceutical composition of claim 2, claim 3, claim 4, claim 5, claim 6, claim 7, claim 8, claim 9, claim 10 or claim 11 to the patient in need thereof.

27. The method of claim 26, which comprises administering pharmaceutical composition of claim 4.

28. The method of claim 27, wherein said pharmaceutical composition is C200 dilution.

29. The method of claim 27, wherein said pharmaceutical composition is prepared by using brain biopotentials of five healthy donors.

30. The method of claim 27, which is in solid form.

31. A method of treating depression in a human patient comprising administering an effective amount of pharmaceutical composition of claim 2, claim 3, claim 4, claim 5, claim 6, claim 7, claim 8, claim 9, claim 10 or claim 11 to the patient in need thereof.

32. The method of claim 31, which comprises administering pharmaceutical composition of claim 4.

33. The method of claim 32, wherein said pharmaceutical composition is C200 dilution.

34. The method of claim 32, wherein said pharmaceutical composition is prepared by using brain biopotentials of five healthy donors.

35. The method of claim 32, which is in solid form.

36. A method of treating attention deficit disorder in a human patient comprising administering an effective amount of pharmaceutical composition of claim 2, claim 3, claim 4, claim 5, claim 6, claim 7, claim 8, claim 9, claim 10 or claim 11 to the patient in need thereof.

37. The method of claim 36, which comprises administering pharmaceutical composition of claim 4.

38. The method of claim 37, wherein said pharmaceutical composition is C200 dilution.

39. The method of claim 37, wherein said pharmaceutical composition is prepared by using brain biopotentials of five healthy donors.

40. The method of claim 37, which is in solid form.

41. A method of treating cerebral palsy in a human patient comprising administering an effective amount of pharmaceutical composition of claim 2, claim 3, claim 4, claim 5, claim 6, claim 7, claim 8, claim 9, claim 10 or claim 11 to the patient in need thereof.

42. The method of claim 41, which comprises administering pharmaceutical composition of claim 4.

43. The method of claim 42, wherein said patient is a child.

44. The method of claim 42, wherein said pharmaceutical composition is C200 dilution.

45. The method of claim 42, wherein said pharmaceutical composition is prepared by using brain biopotentials of five healthy donors.

46. The method of claim 42, which is in solid form.

47. A method of treating stroke consequences in a human patient comprising administering an effective amount of pharmaceutical composition of claim 2, claim 3, claim 4, claim 5, claim 6, claim 7, claim 8, claim 9, claim 10 or claim 11 to the patient in need thereof.

48. The method of claim 47, which comprises administering pharmaceutical composition of claim 4.

49. The method of claim 47, wherein said pharmaceutical composition is C200 dilution.

50. The method of claim 47, wherein said pharmaceutical composition is prepared by using brain biopotentials of five healthy donors.

51. The method of claim 47, which is in solid form.

52. A method of treating anxiety in a human patient comprising administering an effective amount of pharmaceutical composition of claim 2, claim 3, claim 4, claim 5, claim 6, claim 7, claim 8, claim 9, claim 10 or claim 11 to the patient in need thereof.

53. The method of claim 52, which comprises administering pharmaceutical composition of claim 4.

54. The method of claim 52, wherein said pharmaceutical composition is C200 dilution.

55. The method of claim 52, wherein said pharmaceutical composition is prepared by using brain biopotentials of five healthy donors.

56. The method of claim 52, which is in solid form.

57. A method of treating speech disorders in a human patient comprising administering an effective amount of pharmaceutical composition of claim 2, claim 3, claim 4, claim 5, claim 6, claim 7, claim 8, claim 9, claim 10 or claim 11 to the patient in need thereof.

58. The method of claim 57, which comprises administering pharmaceutical composition of claim 4.

59. The method of claim 58, wherein said patient is a child.

60. The method of claim 57, wherein said pharmaceutical composition is C200 dilution.

61. The method of claim 57, wherein said pharmaceutical composition is prepared by using brain biopotentials of five healthy donors.

62. The method of claim 57, which is in solid form.

63. A method of treating autism in a human patient comprising administering an effective amount of pharmaceutical composition of claim 2, claim 3, claim 4, claim 5, claim 6, claim 7, claim 8, claim 9, claim 10 or claim 11 to the patient in need thereof.

64. The method of claim 63, which comprises administering pharmaceutical composition of claim 4.

65. The method of claim 64, wherein said patient is a child.

66. The method of claim 63, wherein said pharmaceutical composition is C200 dilution.

67. The method of claim 63, wherein said pharmaceutical composition is prepared by using brain biopotentials of five healthy donors.

68. The method of claim 63 which is in solid form.

69. A method of treating tension headaches in a human patient comprising administering an effective amount of pharmaceutical composition of claim 2, claim 3, claim 4, claim 5, claim 6, claim 7, claim 8, claim 9, claim 10 or claim 11 to the patient in need thereof.

70. The method of claim 69, which comprises administering pharmaceutical composition of claim 4.

71. The method of claim 69, wherein said pharmaceutical composition is C200 dilution.

72. The method of claim 69 wherein said pharmaceutical composition is prepared by using brain biopotentials of five healthy donors.

73. The method of claim 69, which is in solid form.

74. A method of treating pharmacoresistent epilepsy in a human patient comprising administering an effective amount of pharmaceutical composition of claim 2, claim 3, claim 4, claim 5, claim 6, claim 7, claim 8, claim 9, claim 10 or claim 11 to the patient in need thereof.

75. The method of claim 74, which comprises administering pharmaceutical composition of claim 4.

76. The method of claim 74, wherein said pharmaceutical composition is C200 dilution.

77. The method of claim 74, wherein said pharmaceutical composition is prepared by using brain biopotentials of five healthy donors.

78. The method of claim 74, which is in solid form.

79. A method of treating dementia in a human patient comprising administering an effective amount of pharmaceutical composition of claim 2, claim 3, claim 4, claim 5, claim 6, claim 7, claim 8, claim 9, claim 10 or claim 11 to the patient in need thereof.

80. The method of claim 79, which comprises administering pharmaceutical composition of claim 4.

81. The method of claim 79, wherein said pharmaceutical composition is C200 dilution.

82. The method of claim 79, wherein said pharmaceutical composition is prepared by using brain biopotentials of five healthy donors.

83. The method of claim 79, which is in solid form.

84. A method of improving mental and emotional wellbeing of an animal and/or reducing stress exerted on an animal, said method comprising administering an effective amount of pharmaceutical composition of claim 2, claim 3, claim 4, claim 5, claim 6, claim 7, claim 8, claim 9, claim 10 or claim 11 to the said animal.

85. The method of claim 84, which comprises administering pharmaceutical composition of claim 4.

86. The method of claim 85, wherein said pharmaceutical composition is C200 dilution.

87. The method of claim 85, wherein said pharmaceutical composition is prepared by using brain biopotentials of five healthy human donors.

88. A device used to produce the pharmaceutical composition of claim 1 or claim 2, the device comprising at least two recording electrodes that pick up bioelectric potentials from different brain sites, said electrodes electrically connected to inputs of a channel amplifier of an EEG recording unit, and at least two current electrodes electrically connected to the outputs of said channel amplifier, wherein said two current electrodes are to be placed into a container with liquid carrier to be treated and said at least two recording electrodes to be placed on different sites of a human head.

89. The device of claim 88, wherein one of said recording electrodes is to be connected to an ear-lobe, serving as a reference electrode, and the other active electrode is to be attached to the scalp surface at location(s) overlying different brain sites.

90. The device of claim 89, wherein the reference electrode and one active electrode of each of the referential derivations are connected to the inputs of each channel amplifier of the EEG unit, with the reference outputs of all channel amplifiers connected to a common current electrode and the active outputs linked to the current electrodes of the energy treatment unit, the number of which is equivalent to the number of the active electrodes employed.

91. The device of claim 90, further comprising a low-pass filter with cut-offs of 0.5÷35 Hz connected between the channel amplifier and the analog-to-digital converter.
